SEQUENCE LISTING (119 sequences)

SEQ ID NO: 1 - DNA, 20 bp, artificial sequence
  Preferred termination sequence in yeast
tttttttgttttttatgtct

SEQ ID NO: 2 - DNA, 7240 bp, artificial sequence
  polynucleotide sequence to be integrated in the integration locus on chromosome 14 of S. cerevisiae, comprising the Cas9 expression cassette and the natMX marker and including the 5′- and 3′- flanking regions
ttgagcggacgccgaaacgttgttttttttttgctttagtccagataataaccttttataattttctttttagggaggaagaccggtctaagctcttagaggttctcgcatacccaagtaaaagctaagaccgaagcaaacacgcaggataattttccggtttactctcactgcagctttagtatagatgcagatctggtattgagagaggggggccggaacgaaaagattcaggggagagaaaggcccgggcgtcggggaacaaaaccacttttgcacaccttaattttttagctactctgtttctccgggcttgtatggcacataaataaaattctgctactttccattatctggtcatcacttaccgatgtggagagccttgcccttgaaaaataagataagacctccgattagggtatttgaaattaaccgctgtagaatgaaataaggacgcttgtttgacattttttagttgctttctctatctaccaccatttgcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagaactagtggatcccccgggaaaaatggacaagaagtactccattgggctcgatatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatctcttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgggcgtacgctgcaggtcgacgaattctaccgttcgtataatgtatgctatacgaagttatagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctcatcttgatttatgtcttgtttgaggagatttcggataaatattcttcgaggggaaaatgtcgtaaaaataaaaacaataaagaaacaaaacgattaaaagaaaaactcaaccatcctcaaaagtcctcctcttttttttcacgtgctgcgctgatgtaagcagcaggacatggcaggaagaaaatcggagaattgtggccgcgacacctgacaaacaatcctcaggcaaatgtaaaattgtacatgaaaggctgcgtagtcaatgtcttagaaggccggcccgcatgatccagaagcagaccataaaaaaaataaaaattcgcggtctggcagcaggcataagatgcatagcgttatcctaaatgacatcacgatgataaatcctccgccgcatgatgcttttgatttgcctaagggcctgccatcgtgctggctcaaactattgagggtcaacataccttgaaaatccaagtaaaaggatggatatcgttatactaaaagcaacacagaaaaggtccacgtcagttccacacaataacatttacgtagtgttcacgcgaagcagttacatc

SEQ ID NO: 3 - DNA, 1875 bp, artificial sequence
  polynucleotide sequence of Brick 1 comprising the 2 plasmid
gttttagagctagaaatagcaagttaaaataaggctagtccgttatcaacttgaaaaagtggcaccgagtcggtggtgctttttttgttttttatgtcttcgagtcatgtaattagttatgtcgtggcatgctagctccggattatcgatgataagctgtcaaacatgagaattaattccacggactatagactatacctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaaagcggctgttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgat

SEQ ID NO: 4 - DNA, 2125 bp, artificial sequence
  polynucleotide sequence of Brick 2 comprising the expression cassette of Dasher GFP, flanked upstream by the S. cerevisae TDH3 promoter, and flanked downstream by the S. cerevisiae ADH1 terminator sequence
gttttagagctagaaatagcaagttaaaataaggctagtccgttatcaacttgaaaaagtggcaccgagtcggtggtgctttttttgttttttatgtcttcgagtcatgtaattagttatgtctggccggatcgatgtacacaaccgactgcacccaaacgaacacaaatcttagcagtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaaggttgataaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcagaaagcctgtatgcgaagccacaatcctttccaacagaccatactaagtgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgat

SEQ ID NO: 5 - DNA, 1846 bp, artificial sequence
  polynucleotide sequence of Brick 3 comprising the dominant resistance marker KanMX
gttttagagctagaaatagcaagttaaaataaggctagtccgttatcaacttgaaaaagtggcaccgagtcggtggtgctttttttgttttttatgtcttcgagtcatgtaattagttatgtctgagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgat

SEQ ID NO: 6 - DNA, 2047 bp, artificial sequence
  polynucleotide sequence of Brick 4 comprising the kanR gene, a bacterial selection marker, and pUC ORI
gttttagagctagaaatagcaagttaaaataaggctagtccgttatcaacttgaaaaagtggcaccgagtcggtggtgctttttttgttttttatgtcttcgagtcatgtaattagttatgtctgcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgat

SEQ ID NO: 7 - DNA, 60 bp, artificial sequence
  forward primer gR1-HXT1-f
tgaaagataaatgatgttgtagtcagcgcctctctgttttagagctagaaatagcaagtt

SEQ ID NO: 8 - DNA, 60 bp, artificial sequence
  forward primer gR2-HXT2-f
tgaaagataaatgattgggttaaccttaggacgtcgttttagagctagaaatagcaagtt

SEQ ID NO: 9 - DNA, 60 bp, artificial sequence
  forward primer gR3-HXT3-f
tgaaagataaatgatggtggctagtatttgtttcggttttagagctagaaatagcaagtt

SEQ ID NO: 10 - DNA, 60 bp, artificial sequence
  forward primer gR4-GAL2-f
tgaaagataaatgattctaactctgcctggacggcgttttagagctagaaatagcaagtt

SEQ ID NO: 11 - DNA, 60 bp, artificial sequence
  reverse primer gR1-HXT1-r
ttctagctctaaaacagagaggcgctgactacaacatcatttatctttcactgcggagaa

SEQ ID NO: 12 - DNA, 60 bp, artificial sequence
  reverse primer gR2-HXT2-r
ttctagctctaaaacgacgtcctaaggttaacccaatcatttatctttcactgcggagaa

SEQ ID NO: 13 - DNA, 60 bp, artificial sequence
  reverse primer gR3-HXT3-r
ttctagctctaaaaccgaaacaaatactagccaccatcatttatctttcactgcggagaa

SEQ ID NO: 14 - DNA, 60 bp, artificial sequence
  reverse primer gR4-GAL2-r
ttctagctctaaaacgccgtccaggcagagttagaatcatttatctttcactgcggagaa

SEQ ID NO: 15 - DNA, 1945 bp, artificial sequence
  extended Brick 1
tgaaagataaatgatgttgtagtcagcgcctctctgttttagagctagaaatagcaagttaaaataaggctagtccgttatcaacttgaaaaagtggcaccgagtcggtggtgctttttttgttttttatgtcttcgagtcatgtaattagttatgtcgtggcatgctagctccggattatcgatgataagctgtcaaacatgagaattaattccacggactatagactatacctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaaagcggctgttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgattgggttaaccttaggacgtcgttttagagctagaa

SEQ ID NO: 16 - DNA, 2195 bp, artificial sequence
  extended Brick 2
tgaaagataaatgattgggttaaccttaggacgtcgttttagagctagaaatagcaagttaaaataaggctagtccgttatcaacttgaaaaagtggcaccgagtcggtggtgctttttttgttttttatgtcttcgagtcatgtaattagttatgtctggccggatcgatgtacacaaccgactgcacccaaacgaacacaaatcttagcagtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaaggttgataaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcagaaagcctgtatgcgaagccacaatcctttccaacagaccatactaagtgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatggtggctagtatttgtttcggttttagagctagaa

SEQ ID NO: 17 - DNA, 1916 bp, artificial sequence
  extended Brick 3
tgaaagataaatgatggtggctagtatttgtttcggttttagagctagaaatagcaagttaaaataaggctagtccgttatcaacttgaaaaagtggcaccgagtcggtggtgctttttttgttttttatgtcttcgagtcatgtaattagttatgtctgagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgattctaactctgcctggacggcgttttagagctagaa

SEQ ID NO: 18 - DNA, 2127 bp, artificial sequence
  extended Brick 4
cgcagtgaaagataaatgattctaactctgcctggacggcgttttagagctagaaatagcaagttaaaataaggctagtccgttatcaacttgaaaaagtggcaccgagtcggtggtgctttttttgttttttatgtcttcgagtcatgtaattagttatgtctgcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatgttgtagtcagcgcctctctgttttagagctagaaatagc

SEQ ID NO: 19 - DNA, 382 bp, artificial sequence
  donor DNA used in the CRISPR-CAS9-mediated deletion of genes HXT5-HXT1-HXT4 in S. cerevisiae strain BIE272
tagtcgaacggttctccctctaagaataagaaactatcaaaagaaagagaaaagtcgattgaataatttttctatatataatatacgcaaacaagattcgctttcactttgcaattttacttcatagctttgttaaaaccagcaaaaaatattatttttctagaaaaaagaatatattagaggtaaagaaagatcagttaattaaataatatagacttccttcaaggaataaaaataatatttgatcttcaatttttaacgctttatgaaatatatatttatgatttaatagtttatagataatactttatcaaactacaattttgaattatgttttttgtttcatagttctggaagaggttaggaataggccgaattctcc

SEQ ID NO: 20 - DNA, 468 bp, artificial sequence
  donor DNA used in the CRISPR-CAS9-mediated deletion of gene HXT2 in S. cerevisiae strain BIE272
cggtacgccgcctaacgatagtaataggccacgcaactggcgtggacgacaacaataagtcgcccattttttatgttttcaaaacctagcaacccccaccaaacttgtcatcgttcccggattcacaaatgatataaaaagcgattacaattctacattctaaccagatttgagatttcctctttctcaattcctcttatattagattataagaacaacaaattaaattacaaaaagacttataaagcaacatagagattatacttaaactagcactgatttttttaaggctaatggctactaatactttaatagatgatcttcatacttttttatttaacgatttttaatgatgtttttatttgtaccactcatttatctagatttttttaatactgatcaaatcttacggactcgacgttaaaaagttcctacatacgtctggtacttgaaacgctgcttcgaggt

SEQ ID NO: 21 - DNA, 422 bp, artificial sequence
  donor DNA used in the CRISPR-CAS9-mediated deletion of genes HXT3-HXT6-HXT7 in S. cerevisiae strain BIE272
gactatatccccagagaatttctccagaagagaagaaaagtcaaagttttttttcgcttgggggttgcatataaatacaggcgctgttttatcttcagcatgaatattccataattttacttaatagcttttcataaataatagaatcacaaacaaaatttacatctgagttaaacaatctttgcgaacacttttattaattcatgatcacgctctaatttgtgcatttgaaatgtactctaattctaattttatatttttaatgatatcttgaaaagtaaatacgtttttaatatatacaaaataatacagtttaattttcaagtttttgatcatttgttctcagaaagttgagtgggacggagacaaagaaactttaaagagaaatgcaaagtgggaagaagtcagttgtttaccgaccg

SEQ ID NO: 22 - DNA, 414 bp, artificial sequence
  donor DNA used in the CRISPR-CAS9-mediated deletion of gene GAL2 in S. cerevisiae strain BIE272
tggagataggattagttttgtagacatatataaacaatcagtaattggattgaaaatttggtgttgtgaattgctcttcattatgcaccttattcaattatcatcaagaatagcaatagttaagtaaacacaagattaacataataaaaaaaataattctttcataatggcagttgaggagaacaatatgcctgttgtttcacagcaaccccaagctggtgaagacgtgacctatcgttagaagaaattcaagaattatgggaagaaggtgttttaccttggaaatctgaaggctggattccttcatccagaagaggtaataattacgatttagaggatttacaacatgacgacaaaccgtggtacaaggccatgctagaataatgcgtttgaagtgagacgctccatcatctc

SEQ ID NO: 23 - DNA, 11742 bp, artificial sequence
  CAS9 expression vector pCSN061
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgttgcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgaatcagatccactagtggcctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 24 - DNA, 1609 bp, artificial sequence
  Brick 3b (natMX-fragment)
gttttagagctagaaatagcaagttaaaataaggctagtccgttatcaacttgaaaaagtggcaccgagtcggtggtgctttttttgttttttatgtcttcgagtcatgtaattagttatgtctgagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgat

SEQ ID NO: 25 - DNA, 61 bp, artificial sequence
  forward primer g1-INT59-f
tgaaagataaatgatcagaaaactcttagcttttccgttttagagctagaaatagcaagtt

SEQ ID NO: 26 - DNA, 61 bp, artificial sequence
  forward primer g2-YPRc-f
tgaaagataaatgatccaatatggtatgccgagtctgttttagagctagaaatagcaagtt

SEQ ID NO: 27 - DNA, 61 bp, artificial sequence
  forward primer g3-INT1-f
tgaaagataaatgatctattagaaccagggaggtccgttttagagctagaaatagcaagtt

SEQ ID NO: 28 - DNA, 50 bp, artificial sequence
  forward primer Brick4_to3-f
gccatccagtgtcgaaaacgagctcgcgagcggtatcagctcactcaaag

SEQ ID NO: 29 - DNA, 61 bp, artificial sequence
  reverse primer g1-INT59-r
ttctagctctaaaacggaaaagctaagagttttctgatcatttatctttcactgcggagaa

SEQ ID NO: 30 - DNA, 61 bp, artificial sequence
  reverse primer g2-YPRc-r
ttctagctctaaaacagactcggcataccatattggatcatttatctttcactgcggagaa

SEQ ID NO: 31 - DNA, 61 bp, artificial sequence
  reverse primer g3-INT1-r
ttctagctctaaaacggacctccctggttctaatagatcatttatctttcactgcggagaa

SEQ ID NO: 32 - DNA, 50 bp, artificial sequence
  reverse primer Brick 3_to4-r
ctttgagtgagctgataccgctcgcgagctcgttttcgacactggatggc

SEQ ID NO: 33 - DNA, 1945 bp, artificial sequence
  extended Brick 1 (Example 4)
tgaaagataaatgatagaaaactcttagcttttccgttttagagctagaaatagcaagttaaaataaggctagtccgttatcaacttgaaaaagtggcaccgagtcggtggtgctttttttgttttttatgtcttcgagtcatgtaattagttatgtcgtggcatgctagctccggattatcgatgataagctgtcaaacatgagaattaattccacggactatagactatacctagtatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaaagcggctgttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcaatatggtatgccgagtctgttttagagctagaa

SEQ ID NO: 34 - DNA, 2195 bp, artificial sequence
  extended Brick 2 (Example 4)
tgaaagataaatgatcaatatggtatgccgagtctgttttagagctagaaatagcaagttaaaataaggctagtccgttatcaacttgaaaaagtggcaccgagtcggtggtgctttttttgttttttatgtcttcgagtcatgtaattagttatgtctggccggatcgatgtacacaaccgactgcacccaaacgaacacaaatcttagcagtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaaggttgataaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcagaaagcctgtatgcgaagccacaatcctttccaacagaccatactaagtgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgattattagaaccagggaggtccgttttagagctagaa

SEQ ID NO: 35 - DNA, 1397 bp, artificial sequence
  extended Brick 3b (Example 4)
tgaaagataaatgattattagaaccagggaggtccgttttagagctagaaatagcaagttaaaataaggctagtccgttatcaacttgaaaaagtggcaccgagtcggtggtgctttttttgttttttatgtcttcgagtcatgtaattagttatgtctgagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgcgagcggtatcagctcactcaaag

SEQ ID NO: 36 - DNA, 1983 bp, artificial sequence
  extended Brick 4 (Example 4)
gccatccagtgtcgaaaacgagctcgcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctgaattctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatagaaaactcttagcttttccgttttagagctagaa

SEQ ID NO: 37 - skipped

SEQ ID NO: 38 - DNA, 2540 bp, artificial sequence
  con5 - Low p (KlTDH2p) - crtE - ScTDH3t - conA expression cassette
aagcgacttccaatcgctttgcatatccagtaccacacccacaggcgtttgtgccgtaaaaactaaaacgagcccccaccaaagaacaaaaaagaaggtgctgggcccccactttcttcccttgcacgtgataggaagatggctacagaaacaagaagatggaaatcgaaggaaagagggagactggaagctgtaaaaactgaaatgaaaaaaaaaaaaaaaaaaaaaaacaagaagctgaaaatggaagactgaaatttgaaaaatggtaaaaaaaaaaaagaaacacgaagctaaaaacctggattccattttgagaagaagcaagaaaggtaagtatggtaacgaccgtacaggcaagcgcgaaggcaaatggaaaagctggagtccggaagataatcatttcatcttcttttgttagaacagaacagtggatgtccctcatctcggtaacgtattgtccatgccctagaactctctgtccctaaaaagaggacaaaaacccaatggtttccccagcttccagtggagccaccgatcccactggaaaccactggacaggaagagaaaatcacggacttcctctattgaaggataattcaacactttcaccagatcccaaatgtcccgcccctattcccgtgttccatcacgtaccataacttaccatttcatcacgttctctatggcacactggtactgcttcgactgctttgcttcatcttctctatgggccaatgagctaatgagcacaatgtgctgcgaaataaagggatatctaatttatattattacattataatatgtactagtgtggttattggtaattgtacttaattttgatatataaagggtggatctttttcattttgaatcagaattggaattgcaacttgtctcttgtcactattacttaatagtaattatatttcttattaaccttttttttaagtcaaaacaccaaggacaagaactactcttcaaaggtatttcaagttatcatacgtgtcacacacgcttcacagtttcaagtaaaaaaaaagaatattacacaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatcctcttgcccatcgaacgtacaagtactcctctgttctctccttcctttgcttt

SEQ ID NO: 39 - DNA, 2540 bp, artificial sequence
  con5 - Med p (KlPGK1p) - crtE - ScTDH3t - conA expression cassette
aagcgacttccaatcgctttgcatatccagtaccacacccacaggcgtttgtgcgttcctcatcactagaagccgaactgttgtcttcagtggggattggttcgacattttgccaattgctgtcgatgtaccctttcaaagccatgtaccttaaatcttcatccttggcaagtagattcatcgggtgtgtttgaagtaagaatatttgcttgtttttatggtatcaaaggtatatgttgtagaagacaatttccggtaatccaattgtctgtctgctcagtttagcacatgtatagtacgttgcacatagtctacaatattcagcattcagcattcagtatacagcatatggctaaatgatcacaaatgtgattgatgatttgacacgactagaaaagagaacgaaaaagggaaattccatgtcacgtgcgttggcacgtgacatggaatatcgaagaaagaaaaaaaaaacgatctcgtcctagtggaagcccagagtctggtccccccggagtcttcccaaaacaagaagctgacacatgttgacacagaacaccccacagcaaatgcaccacgctacgtagatcaggaagcttaactctagcgacctgtcgctcgccccacagaacctcacccgagaaccacacattacacgccgccagctcccactatactcatcttgcttcccttaagcgttctcacgattcgttcgctgcccttcttcaagagtcttctgattctaattctcattcgaaatcctctacagttaatgaattgcttgacatgacattcattgtctcatggttttggctttttggcttttgtcttttaaagctatatcaactttacatataaatatacgtcaaaaggggattcattaattagaaaattctctttttcaatagttgctattcattatcaatctattcaactcaattggttattattttcatctttttgtcatcctaaaccatcaacaatatttaaatatatctgttgctacattaagagttacttcagaaataacaaaaaaatcgatcaagaattaataaaaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatcctcttgcccatcgaacgtacaagtactcctctgttctctccttcctttgcttt

SEQ ID NO: 40 - DNA, 2140 bp, artificial sequence
  con5 - Strong p (ScFBA1p) - crtE - ScTDH3t - conA expression cassette
aagcgacttccaatcgctttgcatatccagtaccacacccacaggcgtttgtgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatcctcttgcccatcgaacgtacaagtactcctctgttctctccttcctttgcttt

SEQ ID NO: 41 - DNA, 3431 bp, artificial sequence
  conA - Low p (KlYDRp) - crtYB - ScPDC1t - conB expression cassette
ttgcccatcgaacgtacaagtactcctctgttctctccttcctttgctttgtgcttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcaggtaccattcagattttaatatattcttttctcttcttctacactattattataataattttactatattcatttttagcttaaaacctcatagaatattattcttcagtcactcgcttaaatacttatcaaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctccggatcgatgtacacaaccgactgcacccaaacgaacacaaatcttagca

SEQ ID NO: 42 - DNA, 3431 bp, artificial sequence
  conA - Med p (KlTEF2p) - crtYB - ScPDC1t - conB expression cassette
ttgcccatcgaacgtacaagtactcctctgttctctccttcctttgctttgtgcgagcctgtccaagcaaatgccttctcataaatggtgccaaagacccgcaagcccaaagcaattaccccccaaaaagaaatgatatagtgcaagatacgtatatgaccatgacttgactaggtgaaacagtgcagaaacagccgcacaaaagcagccctaaccctcagagtcgattttactctttcaggtaataaagcctcgacatcaattttagacagaagccaggctggcctcgagattatagccataggcaagcaagaggagagaaggggaggccccccatggggggcctcccccccgctgtcaaggtttggcagaacctagcttcattaggccactagcccagcctaaaacgtcaacgggcaggaggaacactcccacaagacggcgtagtattctcgattcataaccattttctcaatcgaattacacagaacacaccgtacaaacctctctatcataactacttaatagtcacacacgtactcgtctaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctccggatcgatgtacacaaccgactgcacccaaacgaacacaaatcttagca

SEQ ID NO: 43 - DNA, 3031 bp, artificial sequence
  conA - Strong p (ScTEF1p) - crtYB - ScPDC1t - conB expression cassette
ttgcccatcgaacgtacaagtactcctctgttctctccttcctttgctttgtgcttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctccggatcgatgtacacaaccgactgcacccaaacgaacacaaatcttagca

SEQ ID NO: 44 - DNA, 2758 bp, artificial sequence
  conB - Low p (ScPRE3p) - crtI - ScTAL1t - conC expression cassette
cggatcgatgtacacaaccgactgcacccaaacgaacacaaatcttagcagtgccaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtccctcacgctttccggcatcttccagaccacagtatatccatccgcctcctgttg

SEQ ID NO: 45 - DNA, 2758 bp, artificial sequence
  conB - Med p (ScACT1p) - crtI - ScTAL1t - conC expression cassette
cggatcgatgtacacaaccgactgcacccaaacgaacacaaatcttagcagtgcaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtccctcacgctttccggcatcttccagaccacagtatatccatccgcctcctgttg

SEQ ID NO: 46 - DNA, 3158 bp, artificial sequence
  conB - Strong p (KlENO1p) - crtI - ScTAL1t - conC expression cassette
cggatcgatgtacacaaccgactgcacccaaacgaacacaaatcttagcagtgccgtatccctatctggattaacatcactgccacagatcgaattgcaagaagccacacttcacgtgatccactcgttcatcaggtttgtagcttcatggcgcaggacttctgatgatgaactatctggctcatccggatggatcacaaggatggataccctcagacagtacgtttccgtatggagcgatcttacaagaaccaacagttgatcctattactttttttttattttttgtccctccgggatggcaagagggacaaagaagaatcttcgttcttctttcttgttctcaacttcccagcttccgtgtgattaccctccgggacaacagaaaaactggcattcggtatcccgggaatctgctgagaaggaaagaaaacgaaaaaaaaattgtacatttgtgtcacattatgaattacaggaagtcagaaaacaggcagcacatgtctcgcacatgcatgtccatcagacgagacattatgagacatgcacgcgtgtgagagacatagcaaaagtctctccagtacacacagaaagacacgttcacaatccaggcaccccacagagaaaaaaaaaagaagaagcccggaagctggcacgccatcatcaaccaccgctcggtttacacgcatcccaactgtcttttttttctggaatcctataataactggcatctggaaatcacgttgtatgttgcaccatagtgactggctgtctgactagcaaacattgattccctgattcccatttggctcaattttgatgagaaacagttgattgattcttgtcaatttttttttctttggaccaccaccaaccaattgacattgaagtactttcccatgatttgaggttatataaaaggacgttcaaatcactttcaaggttaattcagttttgtcaattgatttaagttcaattgttaacaaatttaatttaattcgaaacaaaccaaaccaattcatttgaattaacaaaccaacccacaaaacaaaaaaaaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtccctcacgctttccggcatcttccagaccacagtatatccatccgcctcctgttg

SEQ ID NO: 47 - DNA, 2534 bp, artificial sequence
  donor expression cassette sequence with 50 bp LF and RF flanks (PCR fragment). Homology to INT1 - Low p (KlTDH2p) - crtE - ScTDH3t - Homology to INT1
catgcctatccggcggcaacattattactctggtatacgggcgtaactccacgtaaaaactaaaacgagcccccaccaaagaacaaaaaagaaggtgctgggcccccactttcttcccttgcacgtgataggaagatggctacagaaacaagaagatggaaatcgaaggaaagagggagactggaagctgtaaaaactgaaatgaaaaaaaaaaaaaaaaaaaaaaaacaagaagctgaaaatggaagactgaaatttgaaaaatggtaaaaaaaaaaaagaaacacgaagctaaaaacctggattccattttgagaagaagcaagaaaggtaagtatggtaacgaccgtacaggcaagcgcgaaggcaaatggaaaagctggagtccggaagataatcatttcatcttcttttgttagaacagaacagtggatgtccctcatctcggtaacgtattgtccatgccctagaactctctgtccctaaaaagaggacaaaaacccaatggtttccccagcttccagtggagccaccgatcccactggaaaccactggacaggaagagaaaatcacggacttcctctattgaaggataattcaacactttcaccagatcccaaatgtcccgcccctattcccgtgttccatcacgtaccataacttaccatttcatcacgttctctatggcacactggtactgcttcgactgctttgcttcatcttctctatgggccaatgagctaatgagcacaatgtgctgcgaaataaagggatatctaatttatattattacattataatatgtactagtgtggttattggtaattgtacttaattttgatatataaagggtggatctttttcattttgaatcagaattggaattgcaacttgtctcttgtcactattacttaatagtaattatatttcttattaaccttttttttaagtcaaaacaccaaggacaagaactactcttcaaaggtatttcaagttatcatacgtgtcacacacgcttcacagtttcaagtaaaaaaaaagaatattacacaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatgaagtatggatctttgaaacccttttattttattttactttttttagaat

SEQ ID NO: 48 - DNA, 2530 bp, artificial sequence
  donor expression cassette sequence with 50 bp LF and RF flanks (PCR fragment). Homology to INT1 - Med p (KlPGK1p) - crtE - ScTDH3t - Homology to INT1
catgcctatccggcggcaacattattactctggtatacgggcgtaactccacctcatcactagaagccgaactgttgtcttcagtggggattggttcgacattttgccaattgctgtcgatgtaccctttcaaagccatgtaccttaaatcttcatccttggcaagtagattcatcgggtgtgtttgaagtaagaatatttgcttgtttttatggtatcaaaggtatatgttgtagaagacaatttccggtaatccaattgtctgtctgctcagtttagcacatgtatagtacgttgcacatagtctacaatattcagcattcagcattcagtatacagcatatggctaaatgatcacaaatgtgattgatgatttgacacgactagaaaagagaacgaaaaagggaaattccatgtcacgtgcgttggcacgtgacatggaatatcgaagaaagaaaaaaaaaacgatctcgtcctagtggaagcccagagtctggtccccccggagtcttcccaaaacaagaagctgacacatgttgacacagaacaccccacagcaaatgcaccacgctacgtagatcaggaagcttaactctagcgacctgtcgctcgccccacagaacctcacccgagaaccacacattacacgccgccagctcccactatactcatcttgcttcccttaagcgttctcacgattcgttcgctgcccttcttcaagagtcttctgattctaattctcattcgaaatcctctacagttaatgaattgcttgacatgacattcattgtctcatggttttggctttttggcttttgtcttttaaagctatatcaactttacatataaatatacgtcaaaaggggattcattaattagaaaattctctttttcaatagttgctattcattatcaatctattcaactcaattggttattattttcatctttttgtcatcctaaaccatcaacaatatttaaatatatctgttgctacattaagagttacttcagaaataacaaaaaaatcgatcaagaattaataaaaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatgaagtatggatctttgaaacccttttattttattttactttttttagaat

SEQ ID NO: 49 - DNA, 2130 bp, artificial sequence
  donor expression cassette sequence with 50 bp LF and RF flanks (PCR fragment). Homology to INT1 - Strong p (ScFBA1p) - crtE - ScTDH3t  -Homology to INT1
catgcctatccggcggcaacattattactctggtatacgggcgtaactccacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatggactacgctaacatcttgactgccattcctttggaattcaccccacaagatgacattgtcttgttggaaccataccactacttaggtaagaacccaggtaaggaaatcagatctcaattgattgaagctttcaactactggttagatgtcaagaaggaagacttggaagttatccaaaatgttgttggtatgttgcacaccgcttctttgttgatggatgatgttgaagattcttccgtcttgagaagaggttctccagttgctcatttgatctacggtattccacaaaccatcaacactgctaactacgtttacttcttggcttaccaagaaatcttcaaattgcgtccaactccaattccaatgccagttatcccaccatcttctgcttctttgcaatcttctgtctcctccgcctcctcttcctcttctgcctcctctgaaaacggtggtacctccactccaaactcccaaatcccattctccaaggacacctacttggacaaggttatcactgacgaaatgttgtctttgcaccgtggtcaaggtttggaattattctggagagactctttgacctgtccatctgaagaagaatacgtcaagatggtcttgggtaagaccggtggtttgttcagaattgctgtcagattgatgatggccaagtctgaatgtgacattgactttgttcaattggttaacttgatttccatctacttccaaatcagagatgactacatgaacttgcaatcctctgaatacgctcacaacaagaacttcgctgaagacttgactgaaggtaagttctccttcccaaccattcactccattcacgctaacccatcttccagattggttatcaacactttacaaaagaagtccacttctccagaaatcttacatcactgtgtcaactacatgagaactgaaacccactctttcgaatacactcaagaagtcttgaacactttatctggtgctttggaaagagaattgggtagattacaaggtgaatttgctgaagctaactccaagatcgatttgggtgacgttgaatctgaaggtagaaccggtaagaacgtcaaattggaagccatcttgaagaaattggctgatatccctctataaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatgaagtatggatctttgaaacccttttattttattttactttttttagaat

SEQ ID NO: 50 - DNA, 3428 bp, artificial sequence
  donor expression cassette sequence with 50 bp LF and RF flanks (PCR fragment). Homology to INT2 - Low p (KlYDR1p) - crtYB - ScPDC1t - Homology to INT2
caaaccgcagattgcgtcacggttctgcggacgccgttagcccattggtgtcaagttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcaggtaccattcagattttaatatattcttttctcttcttctacactattattataataattttactatattcatttttagcttaaaacctcatagaatattattcttcagtcactcgcttaaatacttatcaaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacagaacaatactgattgcgttaccccaagcttatcccacgtagttttgtgtc

SEQ ID NO: 51 - DNA, 3420 bp, artificial sequence
  donor expression cassette sequence with 50 bp LF and RF flanks (PCR fragment). Homology to INT2 - Med p (KlTEF2p) - crtYB - ScPDC1t - Homology to INT2
cgcagattgcgtcacggttctgcggacgccgttagcccattggtgtcaagcctgtccaagcaaatgccttctcataaatggtgccaaagacccgcaagcccaaagcaattaccccccaaaaagaaatgatatagtgcaagatacgtatatgaccatgacttgactaggtgaaacagtgcagaaacagccgcacaaaagcagccctaaccctcagagtcgattttactctttcaggtaataaagcctcgacatcaattttagacagaagccaggctggcctcgagattatagccataggcaagcaagaggagagaaggggaggccccccatggggggcctcccccccgctgtcaaggtttggcagaacctagcttcattaggccactagcccagcctaaaacgtcaacgggcaggaggaacactcccacaagacggcgtagtattctcgattcataaccattttctcaatcgaattacacagaacacaccgtacaaacctctctatcataactacttaatagtcacacacgtactcgtctaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacagaacaatactgattgcgttaccccaagcttatcccacgtagttttgtgtc

SEQ ID NO: 52 - DNA, 3025 bp, artificial sequence
  donor expression cassette sequence with 50 bp LF and RF flanks (PCR fragment). Homology to INT2 - Strong p (ScTEF1p) - crtYB - ScPDC1t - Homology to INT2
caaaccgcagattgcgtcacggttctgcggacgccgttagcccattggtgtcaaggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatgaccgctttggcttactaccaaatccacttgatctacactttgccaatcttaggtttgctaggtttgttgacttctccaattttgaccaaattcgacatctacaagatttctatcttagtctttattgctttctctgctaccactccatgggactcctggatcatcagaaacggtgcctggacctacccatctgctgaatctggtcaaggtgttttcggtacctttttggatgtcccatacgaagaatacgccttctttgttatccaaaccgtcatcaccggtttggtttacgttttggctaccagacatttgttgccatctttggctctaccaaagacccgttcttctgccttgtctctagctttgaaggctttaatcccattgccaatcatctatttgttcaccgctcatccatctccatccccagatcctttggttactgaccactacttctacatgagagctttgtctttgttgatcaccccaccaaccatgttgttggctgctttatctggtgaatacgctttcgactggaaatctggtagagctaagtccaccattgctgccatcatgatcccaactgtctacttgatctgggttgactacgttgccgttggtcaagactcctggtccatcaacgatgaaaagattgtcggttggagattaggtggtgtcttgccaattgaagaagctatgttcttcttattgaccaacttgatgatcgttttgggtttgtctgcctgtgaccacactcaagccttgtacttgttgcacggtagaactatctacggtaacaagaagatgccatcttctttcccattaatcactccaccagttttgtccttgttcttctcctccagaccatactcctcccaaccaaagagagatttggaattggctgtcaagttgttggaaaagaagtccagatctttcttcgttgcttctgccggtttcccatctgaagtcagagaaagattggttggtttgtacgctttctgtcgtgtcaccgatgacttgattgactctccagaagtttcctccaacccacacgctaccattgacatggtttccgatttcttgactttattattcggtcctccattgcacccatctcaaccagacaagattttgtcttctccattattaccaccttcccacccatccagaccaactggtatgtacccattaccaccacctccatctttgtctccagctgaattggtccaattcttgactgaacgtgtcccagttcaataccacttcgctttcagattgttggccaaattgcaaggtttgattccaagatacccattggatgaattattgagaggttacaccactgacttgatcttcccattgtccactgaagccgtccaagctagaaagaccccaattgaaactactgctgacttgttggactacggtttgtgtgttgccggttctgttgctgaattgttggtctacgtttcctgggcttccgctccatcccaagttccagctactattgaagaaagagaagctgttttggtcgcctctcgtgaaatgggtaccgctttgcaattggtcaacattgccagagatatcaagggtgacgctactgaaggtagattctacttgccattgtctttctttggtttgagagatgaatccaaattggccattccaactgactggactgaaccaagacctcaagatttcgacaaattgttgtctctatctccatcttccactttaccatcctctaacgcttctgaatccttcagattcgaatggaagacctactctttgccattggttgcttacgctgaagatttggctaagcactcttacaagggtattgacagattaccaactgaagtccaagctggtatgagagctgcttgtgcttcttacttgttgattggtcgtgaaatcaaggttgtctggaagggtgatgtcggtgaaagaagaaccgttgctggttggagaagagtcagaaaggttttgtctgttgtcatgtccggttgggaaggtcaataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacagaacaatactgattgcgttaccccaagcttatcccacgtagttttgtgtc

SEQ ID NO: 53 - DNA, 2748 bp, artificial sequence
  donor expression cassette sequence with 50 bp LF and RF flanks (PCR fragment). Homology to INT3 - Low p (ScPRE3p) - crtI - ScTAL1t - Homology to INT3
gtccttgtatggaagtatcaaaggggacgttcttcacctccttggaacaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtcgatgggacgtcagcactgtacttgtttttgcgactagattgtaaatcattc

SEQ ID NO: 54 - DNA, 2749 bp, artificial sequence
  donor expression cassette sequence with 50 bp LF and RF flanks (PCR fragment). Homology to INT3 - Med p (ScACT1p) - crtI - ScTAL1t - Homology to INT3
cgtccttgtatggaagtatcaaaggggacgttcttcacctccttggaaaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtcgatgggacgtcagcactgtacttgtttttgcgactagattgtaaatcattc

SEQ ID NO: 55 - DNA, 3149 bp, artificial sequence
  donor expression cassette sequence with 50 bp LF and RF flanks (PCR fragment). Homology to INT3 - Strong p (KlENO1p) - crtI - ScTAL1t - Homology to INT3
cgtccttgtatggaagtatcaaaggggacgttcttcacctccttggaacgtatccctatctggattaacatcactgccacagatcgaattgcaagaagccacacttcacgtgatccactcgttcatcaggtttgtagcttcatggcgcaggacttctgatgatgaactatctggctcatccggatggatcacaaggatggataccctcagacagtacgtttccgtatggagcgatcttacaagaaccaacagttgatcctattactttttttttattttttgtccctccgggatggcaagagggacaaagaagaatcttcgttcttctttcttgttctcaacttcccagcttccgtgtgattaccctccgggacaacagaaaaactggcattcggtatcccgggaatctgctgagaaggaaagaaaacgaaaaaaaaattgtacatttgtgtcacattatgaattacaggaagtcagaaaacaggcagcacatgtctcgcacatgcatgtccatcagacgagacattatgagacatgcacgcgtgtgagagacatagcaaaagtctctccagtacacacagaaagacacgttcacaatccaggcaccccacagagaaaaaaaaaagaagaagcccggaagctggcacgccatcatcaaccaccgctcggtttacacgcatcccaactgtcttttttttctggaatcctataataactggcatctggaaatcacgttgtatgttgcaccatagtgactggctgtctgactagcaaacattgattccctgattcccatttggctcaattttgatgagaaacagttgattgattcttgtcaatttttttttctttggaccaccaccaaccaattgacattgaagtactttcccatgatttgaggttatataaaaggacgttcaaatcactttcaaggttaattcagttttgtcaattgatttaagttcaattgttaacaaatttaatttaattcgaaacaaaccaaaccaattcatttgaattaacaaaccaacccacaaaacaaaaaaaaatgggtaaggaacaagaccaagacaagccaactgccatcatcgttggttgtggtatcggtggtattgctaccgctgccagattagctaaggaaggtttccaagttaccgtctttgaaaagaacgactactccggtggtagatgttctttgattgaaagagatggttacagattcgaccaaggtccatctttgttgctattaccagacttgttcaagcaaaccttcgaagatttgggtgaaaagatggaagactgggttgatttgatcaagtgtgaaccaaactacgtttgtcacttccatgatgaagaaactttcaccttctccactgacatggctttattgaagagagaagtcgaaagatttgaaggtaaagatggtttcgacagattcttgtctttcatccaagaagctcacagacattacgaattggctgttgtccacgtcttgcaaaagaacttcccaggtttcgctgctttcttgagattacaattcatcggtcaaatcttagctttgcacccatttgaatccatctggaccagagtttgtcgttacttcaagactgacagattgagaagagtcttctcctttgccgttatgtacatgggtcaatctccatactctgctccaggtacctactccttgttgcaatacactgaattgactgaaggtatctggtacccaagaggtggtttctggcaagttccaaacactttgttgcaaatcgtcaagagaaacaacccatctgctaagttcaacttcaacgctccagtttctcaagttttgttgtctccagctaaggacagagctaccggtgtcagattagaatctggtgaagaacaccacgctgatgttgtcattgtcaatgctgacttggtctacgcttctgaacatttgattccagatgatgctagaaacaagatcggtcaattaggtgaagttaagcgttcctggtgggctgatttggttggtggtaagaagttgaagggttcttgttcttctttgtctttctactggtctatggacagaatcgttgacggtttgggtggtcacaacatcttcttggctgaagacttcaagggttccttcgacaccattttcgaagaattgggtttgccagctgacccatctttctatgttaacgttccatccagaattgacccttctgctgctccagaaggtaaggatgccattgtcatcttagtcccatgtggtcacatcgatgcttccaaccctcaagactacaacaaattggttgccagagccagaaagttcgtcatccaaaccttgtctgccaagttgggtctaccagatttcgaaaagatgattgttgctgaaaaggttcacgatgctccatcctgggaaaaggaattcaacttgaaggacggttccattttgggtttggctcacaacttcatgcaagtcttgggtttcagaccatccaccagacacccaaagtacgacaaattgttctttgtcggtgcttctacccacccaggtactggtgttccaattgtcttggctggtgccaaattgactgctaaccaagttttggaatccttcgatcgttctccagctccagatcctaacatgtctttgtctgttccatacggtaagccattgaaatccaacggtactggtattgactctcaagtccaattgaaattcatggacttggaacgttgggtttacctattagtcttgttgattggtgctgttatcgccagatccgtcggtgtcttggccttttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtcgatgggacgtcagcactgtacttgtttttgcgactagattgtaaatcattc

SEQ ID NO: 56 - DNA, 71 bp, artificial sequence
  forward primer to obtain SEQ ID NO: 47
catgcctatccggcggcaacattattactctggtatacgggcgtaactccacgtaaaaactaaaacgagcc

SEQ ID NO: 57 - DNA, 74 bp, artificial sequence
  reverse primer to obtain SEQ ID NO: 47, 48, 49
attctaaaaaaagtaaaataaaataaaagggtttcaaagatccatacttcatcctggcggaaaaaattcatttg

SEQ ID NO: 58 - DNA, 72 bp, artificial sequence
  forward primer to obtain SEQ ID NO: 48
catgcctatccggcggcaacattattactctggtatacgggcgtaactccacctcatcactagaagccgaac

SEQ ID NO: 59 - DNA, 71 bp, artificial sequence
  forward primer to obtain SEQ ID NO: 49
catgcctatccggcggcaacattattactctggtatacgggcgtaactccacttggcttcacatacgttgc

SEQ ID NO: 60 - DNA, 84 bp, artificial sequence
  forward primer to obtain SEQ ID NO: 50
caaaccgcagattgcgtcacggttctgcggacgccgttagcccattggtgtcaagttttctttttttgcggtcacccccatgtg

SEQ ID NO: 61 - DNA, 70 bp, artificial sequence
  reverse primer to obtain SEQ ID NO: 50, 51, 52
gacacaaaactacgtgggataagcttggggtaacgcaatcagtattgttctgttccttaatcaaggatac

SEQ ID NO: 62 - DNA, 73 bp, artificial sequence
  forward primer to obtain SEQ ID NO: 51
cgcagattgcgtcacggttctgcggacgccgttagcccattggtgtcaagcctgtccaagcaaatgccttctc

SEQ ID NO: 63 - DNA, 80 bp, artificial sequence
  forward primer to obtain SEQ ID NO: 52
caaaccgcagattgcgtcacggttctgcggacgccgttagcccattggtgtcaaggctgataatagcgtataaacaatgc

SEQ ID NO: 64 - DNA, 71 bp, artificial sequence
  forward primer to obtain SEQ ID NO: 53
gtccttgtatggaagtatcaaaggggacgttcttcacctccttggaacaaacattaatttgttctgcatac

SEQ ID NO: 65 - DNA, 72 bp, artificial sequence
  reverse primer to obtain SEQ ID NO: 53, 54, 55
gaatgatttacaatctagtcgcaaaaacaagtacagtgctgacgtcccatcgacgttgatttaaggtggttc

SEQ ID NO: 66 - DNA, 73 bp, artificial sequence
  forward primer to obtain SEQ ID NO: 54
cgtccttgtatggaagtatcaaaggggacgttcttcacctccttggaaaacatatatacacaattacagtaac

SEQ ID NO: 67 - DNA, 74 bp, artificial sequence
  forward primer to obtain SEQ ID NO: 55
cgtccttgtatggaagtatcaaaggggacgttcttcacctccttggaacgtatccctatctggattaacatcac

SEQ ID NO: 68 - DNA, 584 bp, artificial sequence
  right flank sequence with overlap ScTDH3t - INT1 3′ (part of donor) - INT1 3′ genomic DNA. Homology with INT1 3′ genomic DNA 524 bp
aattttggggatattggcttttttttttaaagtttacaaatgaattttttccgccaggatgaagtatggatctttgaaacccttttattttattttactttttttagaatgacctgttcccgacactatgtaagatctagcttttaacatattatggaaacctgaaatgtaaaatctgaatttttgtatatgtgtttatatttgggtagttcttttgaggaaagcatgcatagacttgctgtacgaactttatgtgacttgtagtgacgctgtttcatgagactttagccctttgaacatattatcatatctcagcttgaaatactatagatttacttttgcagccatttcttggtgctccaaggttgtgcgtatctattacttaatttctgtccttgccaagttttgcagcagggcggtcacaagactcctctgccgtcattccttagtccttcgggaacacacttatttatgtatttgtattctacaattctacggtgcacaagggttgggcactgttgagctcagcacgcaactattgctggcatgaagataagattgatttttggaagaataagcttgtg

SEQ ID NO: 69 - DNA, 372 bp, artificial sequence
  left flank sequence with overlap KlYDR2p - INT2 5′ (part of donor) - INT2 5′ genomic DNA. Homology with INT2 5′ genomic DNA 322 bp
cattatatcgaggaaagccctgcgaacgtgaagtgaaaaggacggttgtcctttttcctgttcttcgaggatcctgaacaggatgtcagtagggcgggccttttctttttattgtgcaggtacgcatgtttctttgacacaaaatagtctggattcgccagccagtggcacttttgatgccatttccaccacagaaaacgtctgagtattcagccatgggaggggagtgttgaaaggggagaagcagcccacttctctgtcttgacacaaaccgcagattgcgtcacggttctgcggacgccgttagcccattggtgtcaagttttctttttttgcggtcacccccatgtggcggggaggcagaggagtagg

SEQ ID NO: 70 - DNA, 372 bp, artificial sequence
  left flank sequence with overlap KlTEF2p - INT2 5′ (part of donor) - INT2 5′ genomic DNA. Homology with INT2 5′ genomic DNA 322 bp
cattatatcgaggaaagccctgcgaacgtgaagtgaaaaggacggttgtcctttttcctgttcttcgaggatcctgaacaggatgtcagtagggcgggccttttctttttattgtgcaggtacgcatgtttctttgacacaaaatagtctggattcgccagccagtggcacttttgatgccatttccaccacagaaaacgtctgagtattcagccatgggaggggagtgttgaaaggggagaagcagcccacttctctgtcttgacacaaaccgcagattgcgtcacggttctgcggacgccgttagcccattggtgtcaagcctgtccaagcaaatgccttctcataaatggtgccaaagacccgcaagcc

SEQ ID NO: 71 - DNA, 373 bp, artificial sequence
  left flank sequence with overlap ScTEF1p - INT2 5′ (part of donor) - INT2 5′ genomic DNA. Homology with INT2 5′ genomic DNA 322 bp
cattatatcgaggaaagccctgcgaacgtgaagtgaaaaggacggttgtcctttttcctgttcttcgaggatcctgaacaggatgtcagtagggcgggccttttctttttattgtgcaggtacgcatgtttctttgacacaaaatagtctggattcgccagccagtggcacttttgatgccatttccaccacagaaaacgtctgagtattcagccatgggaggggagtgttgaaaggggagaagcagcccacttctctgtcttgacacaaaccgcagattgcgtcacggttctgcggacgccgttagcccattggtgtcaaggctgataatagcgtataaacaatgcatactttgtacgttcaaaatacaatg

SEQ ID NO: 72 - DNA, 608 bp, artificial sequence
  right flank sequence with overlap ScPDC1t - INT2 3′ (part of donor) - INT2 3′ genomic DNA. Homology with INT2 3′ genomic DNA 524 bp
gaattagtggaaccaaggaaaaaaaaagaggtatccttgattaaggaacagaacaatactgattgcgttaccccaagcttatcccacgtagttttgtgtctctttcttgggctcttggcggccgtacgcatatagtatactaatgcggtaacggctacctcctacgtctgtatcaaaaaagttcgctctggtcctatcaatattttctcccacacgaggattctgtggttttgcagccacatctacaggatgttgactttttgtttgtttacgtagtttgtttcaacgctttcgaagatgtggcgctggtgcaagtcatctaagttggttactgataacaaattgtgacaaggatgtagtggatgcgtaggagaccaaaatgacatctgacacaaaaaaaagtagataacgaaaacaggacaaaggtgtcattttgttttgccaaggagaattataatcttagtgtgaagatactttcattagacacataaatttacgttcgacacacttttttcttactcctccccttatcaaggatacctggttgattctacgtcgtcctcttcactttggttaattcacctttgcccttcacctcgtggtgtgcg

SEQ ID NO: 73 - DNA, 649 bp, artificial sequence
  left flank sequence with overlap ScPRE3p - INT3 5′ (part of donor) - INT3 5′ genomic DNA. Homology with INT3 5′ genomic DNA 602 bp
taaaggaggtgcacgcattatggagaccactacgatacgatagctgcgttgttgttgaaggggtttcttaaggttgttttcgttgaaggtaaatattggtcgtttttgtgcagcatattgtcctctagatgcaaactctgcaggtccatttgcagtaaagtgagttgcctctcgaagaatcattaatttcgtataaccgtcactattaaagtcagaaaataaattctgtcgtagacaatgttaccataatgttcttgtccattttgcatacactttaaatattcatttgatttctcagggttcatgatcataataaattgcgcattcgcaaggcggtagtattataatggggtccatcattctgtagcaagaagttacagtacgctgttcaagcgttaaacaagataagtaatctcgaatgaaacattcatatttcgcatgagccaacatacagttgctgagtaatcttcattgcgcttatttatcggcattgagattgtaaaggaagtaaaacgcatttttgcagatctgttctcttatgtatttttaatcgtccttgtatggaagtatcaaaggggacgttcttcacctccttggaacaaacattaatttgttctgcatactttgaacctttcagaaaataaaaaac

SEQ ID NO: 74 - DNA, 648 bp, artificial sequence
  left flank sequence with overlap ScACT1p - INT3 5′ (part of donor) - INT3 5′ genomic DNA. Homology with INT3 5′ genomic DNA 602 bp
taaaggaggtgcacgcattatggagaccactacgatacgatagctgcgttgttgttgaaggggtttcttaaggttgttttcgttgaaggtaaatattggtcgtttttgtgcagcatattgtcctctagatgcaaactctgcaggtccatttgcagtaaagtgagttgcctctcgaagaatcattaatttcgtataaccgtcactattaaagtcagaaaataaattctgtcgtagacaatgttaccataatgttcttgtccattttgcatacactttaaatattcatttgatttctcagggttcatgatcataataaattgcgcattcgcaaggcggtagtattataatggggtccatcattctgtagcaagaagttacagtacgctgttcaagcgttaaacaagataagtaatctcgaatgaaacattcatatttcgcatgagccaacatacagttgctgagtaatcttcattgcgcttatttatcggcattgagattgtaaaggaagtaaaacgcatttttgcagatctgttctcttatgtatttttaatcgtccttgtatggaagtatcaaaggggacgttcttcacctccttggaaaacatatatacacaattacagtaacaataacaagaggacagatactacc

SEQ ID NO: 75 - DNA, 649 bp, artificial sequence
  left flank sequence with overlap KlENO1p - INT3 5′ (part of donor) - INT3 5′ genomic DNA. Homology with INT3 5′ genomic DNA 602 bp
taaaggaggtgcacgcattatggagaccactacgatacgatagctgcgttgttgttgaaggggtttcttaaggttgttttcgttgaaggtaaatattggtcgtttttgtgcagcatattgtcctctagatgcaaactctgcaggtccatttgcagtaaagtgagttgcctctcgaagaatcattaatttcgtataaccgtcactattaaagtcagaaaataaattctgtcgtagacaatgttaccataatgttcttgtccattttgcatacactttaaatattcatttgatttctcagggttcatgatcataataaattgcgcattcgcaaggcggtagtattataatggggtccatcattctgtagcaagaagttacagtacgctgttcaagcgttaaacaagataagtaatctcgaatgaaacattcatatttcgcatgagccaacatacagttgctgagtaatcttcattgcgcttatttatcggcattgagattgtaaaggaagtaaaacgcatttttgcagatctgttctcttatgtatttttaatcgtccttgtatggaagtatcaaaggggacgttcttcacctccttggaacgtatccctatctggattaacatcactgccacagatcgaattgcaagaag

SEQ ID NO: 76 - DNA, 674 bp, artificial sequence
  right flank RF sequence with overlap ScTAL1t - INT3 3′ (part of donor) - INT3 3′ genomic DNA. Homology with INT3 3′ genomic DNA 624 bp
gcagcccctaagatcagacaaaacatccggaaccaccttaaatcaacgtcgatgggacgtcagcactgtacttgtttttgcgactagattgtaaatcattctttatttaatctctttctttaactactgcttaaagtataatttggtccgtagtttaataactatactaagcgtaacaatgcatactgacattataagcctgaacattacgagtttaagttgtatgtaggcgttctgtaagaggttactgcgtaaattatcaacgaatgcattggtgtatttgcgaaagctacttcttttaacaagtatttacataagaataatggtgatctgctcaactgatttggtgataactctaacttttttagcaacaatttaaaagataattcgaacatatataacagtaggaagaatttgtgtacgtcaaattaagataatttagcattaccaaagttattaacctaaacataaaatatatatgagacacatgtggaaatcgtatgaaacaactgttatgaaactgacaagaatgaatatatagagtaagctccgcttgtaaagaggaatcacttaagtgtataaatgtctcgacgattactttagatccaagattgatgattgatattactctgtaatacttaagctcttttaatagctcactgttgtattacggg

SEQ ID NO: 77 - DNA, 81 bp, artificial sequence
  forward primer primer to obtain SEQ ID NO: 68
aattttggggatattggcttttttttttaaagtttacaaatgaattttttccgccaggatgaagtatggatctttgaaacc

SEQ ID NO: 78 - DNA, 24 bp, artificial sequence
  reverse primer to obtain SEQ ID NO: 68
cacaagcttattcttccaaaaatc

SEQ ID NO: 79 - DNA, 20 bp, artificial sequence
  forward primer to obtain SEQ ID NO: 69, 70 ,71
cattatatcgaggaaagccc

SEQ ID NO: 80 - DNA, 71 bp, artificial sequence
  reverse primer to obtain SEQ ID NO: 69
cctactcctctgcctccccgccacatgggggtgaccgcaaaaaaagaaaacttgacaccaatgggctaacg

SEQ ID NO: 81 - DNA, 71 bp, artificial sequence
  reverse primer to obtain SEQ ID NO: 70
ggcttgcgggtctttggcaccatttatgagaaggcatttgcttggacaggcttgacaccaatgggctaacg

SEQ ID NO: 82 - DNA, 72 bp, artificial sequence
  reverse primer to obtain SEQ ID NO: 71
cattgtattttgaacgtacaaagtatgcattgtttatacgctattatcagccttgacaccaatgggctaacg

SEQ ID NO: 83 - DNA, 72 bp, artificial sequence
  forward primer to obtain SEQ ID NO: 72
gaattagtggaaccaaggaaaaaaaaagaggtatccttgattaaggaacagaacaatactgattgcgttacc

SEQ ID NO: 84 - DNA, 20 bp, artificial sequence
  reverse primer to obtain SEQ ID NO: 72
cgcacaccacgaggtgaagg

SEQ ID NO: 85 - DNA, 23 bp, artificial sequence
  forward primer to obtain SEQ ID NO: 73, 74, 75
taaaggaggtgcacgcattatgg

SEQ ID NO: 86 - DNA, 72 bp, artificial sequence
  reverse primer to obtain SEQ ID NO: 73
gttttttattttctgaaaggttcaaagtatgcagaacaaattaatgtttgttccaaggaggtgaagaacgtc

SEQ ID NO: 87 - DNA, 71 bp, artificial sequence
  reverse primer to obtain SEQ ID NO: 74
ggtagtatctgtcctcttgttattgttactgtaattgtgtatatatgttttccaaggaggtgaagaacgtc

SEQ ID NO: 88 - DNA, 72 bp, artificial sequence
  reverse primer to obtain SEQ ID NO: 75
cttcttgcaattcgatctgtggcagtgatgttaatccagatagggatacgttccaaggaggtgaagaacgtc

SEQ ID NO: 89 - DNA, 71 bp, artificial sequence
  forward primer to obtain SEQ ID NO: 76
gcagcccctaagatcagacaaaacatccggaaccaccttaaatcaacgtcgatgggacgtcagcactgtac

SEQ ID NO: 90 - DNA, 20 bp, artificial sequence
  reverse primer to obtain SEQ ID NO: 76
cccgtaatacaacagtgagc

SEQ ID NO: 91 - DNA, 5712 bp, artificial sequence
  vector pRN1120
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 92 - DNA, 21 bp, artificial sequence
  forward primer to remove SapI restriction site in pRN1120
tattgggccctcttccgcttc

SEQ ID NO: 93 - DNA, 18 bp, artificial sequence
  reverse primer to remove SapI restriction site in pRN1120
cgcaaaccgcctctcccc

SEQ ID NO: 94 - DNA, 547 bp, artificial sequence
  gBlock allowing direct SapI cloning of the guide sequence, part of vector pGRN002
ggagctagcatgcggccgctctagaactagtggatcccccgggctgcaggaattctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaagagcctgaggtcgacggtatcgataagcttgatatcaattccccgggggatccactgctcttctgttttagagctagaaatagcaagttaaaataaggctagtccgttatcaacttgaaaaagtggcaccgagtcggtggtgctttttttgttttttatgtctctcgagggggggcccggtacccagcttttgttccctttagtgagggttaattccga

SEQ ID NO: 95 - DNA, 6121 bp, artificial sequence
  expression vector pGRN002
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcagaagagcctgaggtcgacggtatcgataagcttgatatcaattccccgggggatccactgctcttctgttttagagctagaaatagcaagttaaaataaggctagtccgttatcaacttgaaaaagtggcaccgagtcggtggtgctttttttgttttttatgtctctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 96 - DNA, 53 bp, artificial sequence
  forward primer approach 1: 30 bp on either side
ataaatgatctattagaaccagggaggtccgttttagagctagaaatagcaag

SEQ ID NO: 97 - DNA, 52 bp, artificial sequence
  reverse primer approach 1: 30 bp on either side
gctctaaaacggacctccctggttctaatagatcatttatctttcactgcgg

SEQ ID NO: 98 - DNA, 6113 bp, artificial sequence
  PCR fragment approach 1: 30 bp on eiter side. The fragment was transformed to yeast allowing reconstitution of a circular vector by in vivo recombination
ataaatgatctattagaaccagggaggtccgttttagagctagaaatagcaagttaaaataaggctagtccgttatcaacttgaaaaagtggcaccgagtcggtggtgctttttttgttttttatgtctctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagc

SEQ ID NO: 99 - DNA, 73 bp, artificial sequence
  forward primer approach 1: 50 bp tail on one side
aaacttctccgcagtgaaagataaatgatctattagaaccagggaggtccgttttagagctagaaatagcaag

SEQ ID NO: 100 - DNA, 22 bp, artificial sequence
  reverse primer approach 1: 50 bp tail on one side
gatcatttatctttcactgcgg

SEQ ID NO: 101 - DNA, 6103 bp, artificial sequence
  PCR fragment approach 2: 50 bp tail on one side. The fragment was transformed to yeast allowing reconstitution of a circular vector by in vivo recombination
aaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatc

SEQ ID NO: 102 - DNA, 23 bp, artificial sequence
  forward primer to obtain the 5′ flank A - connector 5 sequence to obtain 1 kB deletion of genomic DNA
cactatagcaatctggctatatg

SEQ ID NO: 103 - DNA, 24 bp, artificial sequence
  reverse primer to obtain the 5′ flank A and B - connector 5 sequence to obtain 1 kB deletion of genomic DNA
aaacgcctgtgggtgtggtactgg

SEQ ID NO: 104 - DNA, 500 bp, artificial sequence
  fragment A (5′ flank A - connector 5) to obtain 1 kB deletion of genomic DNA
cactatagcaatctggctatatgatatgcagaaaatagtgattactgtgctctttgctgcatcatgcatcatatgtttggcattctgtcctgtcagatgaatgggcgaaacaattcggggagctttgttgcgacttggtacccggcaagccgcgacctatattttgcattaaacgagttcatcgtggatttatcgccatgagccttagcttatcaagcctctcacagacaattaagcaatgaaaaaggtataccatcggcgcagaatggttaactagtgggttcatactgctgtgttatagattgttacctaagtgatcaccaaaaaaaagtgcaaaaaggaaaaaaaaataagagacaggtaacttccacaagcttattcttccaaaaatcaatcttatcttcatgccagcaatagttgcgtgctgagctcaacagtgcccaacccttgaagcgacttccaatcgctttgcatatccagtaccacacccacaggcgttt

SEQ ID NO: 105 - DNA, 26 bp, artificial sequence
  forward primer to obtain the 5′ flank B - connector 5 sequence to obtain 10 kB deletion of genomic DNA
caatagtaattttaaaaacaatgtgc

SEQ ID NO: 106 - DNA, 500 bp, artificial sequence
  fragment B (5′ flank B - connector 5) to obtain 10 kB deletion of genomic DNA
caatagtaattttaaaaacaatgtgcttatccagaaattccataaggagtagcgcttggatccctcacctttttcggtcgcggcagtcttcaaaaccttttttggctcgtcatttttttttaactttgccttctttgtcacagccatattttgcctcgaacgatcacctttcatcgtaggctatccaatagacgtcaaatgccttgctgaccactacagaactgtaatgaacttcagctatttttaacgtatgttattctataattctataccactattgtcgttttgaaaaatgaaatttcacttttcggaagttgtccgctaagaacaactaagtgataacagtaaattttaaaaatgctttgaaaaatgggttaagcaaaggttatttactataactacgtagtgaacgaaaccatatatctcttaatttgcagcagggattttataaagcgacttccaatcgctttgcatatccagtaccacacccacaggcgttt

SEQ ID NO: 107 - DNA, 22 bp, artificial sequence
  forward primer to obtain Connector 5 - TDH3p - GFP - ENO1t - Connector 3 PCR fragment
aagcgacttccaatcgctttgc

SEQ ID NO: 108 - DNA, 24 bp, artificial sequence
  reverse primer to obtain Connector 5 - TDH3p - GFP - ENO1t - Connector 3 PCR fragment
acttagtatggtctgttggaaagg

SEQ ID NO: 109 - DNA, 1726 bp, artificial sequence
  fragment C, Connector 5 - TDH3p - GFP - ENO1t - Connector 3 synthetic cassette
aagcgacttccaatcgctttgcatatccagtaccacacccacaggcgtttgtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaattgatttgtactactggtaaattgccagttccatggccaaccttagtcactactttaggttatggtttgcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcactgctgacaaacaaaagaatggtatcaaagctaacttcaaaattagacacaacattgaagatggtggtgttcaattagctgaccattatcaacaaaatactccaattggtgatggtccagtcttgttaccagacaaccattacttatcctatcaatctgccttatccaaagatccaaacgaaaagagagatcacatggtcttgttagaatttgttactgctgctggtattacccatggtatggatgaattgtacaaataaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctcagaaagcctgtatgcgaagccacaatcctttccaacagaccatactaagt

SEQ ID NO: 110 - DNA, 72 bp, artificial sequence
  forward primer to obtain the connector 3 - 3′ flank A sequence to obtain 1 kB deletion of genomic DNA
agaaagcctgtatgcgaagccacaatcctttccaacagaccatactaagtaagcgttgaagtttcctctttg

SEQ ID NO: 111 - DNA, 20 bp, artificial sequence
  reverse primer to obtain the connector 3 - 3′ flank A and B sequence to obtain 1 kB deletion of genomic DNA
tgtcaactggagagctatcg

SEQ ID NO: 112 - DNA, 581 bp, artificial sequence
  fragment D (connector 3 - 3′ flank A) to obtain 1 kB deletion of genomic DNA
agaaagcctgtatgcgaagccacaatcctttccaacagaccatactaagtaagcgttgaagtttcctctttgtatatttgagatcttcattttatcggattctttgtcatcagacaacttgttgagtggtactaaaggagtgcttttcatcatccttttggtgaacgatttcaaatacgttagtgttttctgagctagttttgatcaattcaggtgattcgttatcagaactctcaggtttgtattcgtgtccagttgtgtagcattcgcctaacgtgtaagcacggatttcttcctcagaaatttcactgtatggaatcatgcccttctttctcgcttcttcgtcggtaaatgcaccatagtaatctttgtcatcatgtctaacagtaattttgaatgggaagaagacacatagcccccagtaaacgaaaaaagaaatcaaaaaggagaagaaagaatcaccataaaagaatttaacaatacctgagtcgtggaaatagttattgttgacttcccaagcgataccaggtagaccgggagccataccacacacccaggcaacgatagctctccagttgaca

SEQ ID NO: 113 - DNA, 71 bp, artificial sequence
  forward primer to obtain the connector 3 - 3′ flank B sequence to obtain 10 kB deletion of genomic DNA
agaaagcctgtatgcgaagccacaatcctttccaacagaccatactaagtctgttgtgacagcatcttgtc

SEQ ID NO: 114 - DNA, 22 bp, artificial sequence
  reverse primer to obtain the connector 3 - 3′ flank B sequence to obtain 10 kB deletion of genomic DNA
cctagcagtagttgtaatgtgg

SEQ ID NO: 115 - DNA, 607 bp, artificial sequence
  fragment E (connector 3 - 3′ flank B) to obtain 10 kB deletion of genomic DNA
agaaagcctgtatgcgaagccacaatcctttccaacagaccatactaagtctgttgtgacagcatcttgtctattatttattggatacgcaaaactcatttttttaaaattttttttataatttcctttaaataaaaaggttaaagctgggaaaaatgcatctaaataataaactaatgatttagtttccctcatttaaaatatctaagaaggacagaactttgtttggggtacttacccttgagtatttttagcgattattcttggagttctcagccagaagattaaaagaatatatgttactgaatgcagtgtgatactcaagactaagtagtcttaacaacaagttaacaagggcgtgtggtctagtggtatgattctcgctttgggcgacttcctgactaacaggaagacaaagcatgcgagaggccctgggttcaattcccagctcgccccttttatttttgattatttatttcctattggtcgggtaacagattttttttatttttttcaaggcccttttgttttgtaaacaaagaaatttcacaaagtgagcgtctactaatattgataatgccatttccacattacaactactgctagg

SEQ ID NO: 116 - DNA, 15 bp, artificial sequence
  polynucleotide sequence of the first 15 polynucleotides (nucleotide 1-15) in the forward primers according to SEQ ID NO: 7 to 10
tgaaagataaatgat

SEQ ID NO: 117 - DNA, 25 bp, artificial sequence
  polynucleotide sequence of the last 25 polynucleotides (nucleotide 36-60) in the forward primers according to SEQ ID NO: 7 to 10
gttttagagctagaaatagcaagtt

SEQ ID NO: 118 - DNA, 15 bp, artificial sequence
  polynucleotide sequence of the first 15 polynucleotides (nucleotide 1-15) in the reverse primers according to SEQ ID NO: 11 to 14
ttctagctctaaaac

SEQ ID NO: 119 - DNA, 25 bp, artificial sequence
  polynucleotide sequence of the last 25 polynucleotides (nucleotide 36-60) in the reverse primers according to SEQ ID NO: 11 to 14
atcatttatctttcactgcggagaa

(19) United States
(12) Patent Application Publication
(10) Pub. No.: US 2019/0055544 A1
(43) Pub. Date: Feb. 21, 2019

(54) GUIDE RNA ASSEMBLY VECTOR

(71) Applicant: DSM IP ASSETS B.V., Heerlen (NL)

(72) Inventors: Paulus Petrus DE WAAL, Echt (NL); Johannes Andries ROUBOS, Echt (NL); Paul KLAASSEN, Echt (NL); René VERWAAL, Echt (NL)

(21) Appl. No.: 15/741,985

(22) PCT Filed: Jul. 6, 2016
(86) PCT No.: PCT/EP2016/066016

§ 371 Date: Jan. 4, 2018

(30) Foreign Application Priority Data
Jul. 6, 2015 (EP) 15175444.7

(51) Int. Cl. C12N 15/10; C12N 15/62; C12N 15/81; C12N 15/64; C12Q 1/6806; C12Q 1/686

(52) U.S. Cl. CPC C12N 15/1031; C12N 15/62; C12N 15/81; C12N 2310/20; C12Q 1/6806; C12Q 1/686; C12N 15/64

(57) ABSTRACT

The present invention relates relates to a CRISPR-CAS system for a host cell, in particular to a method to produce a circular vector comprising one or more guide-polynucleotide expression cassettes, wherein said one or more guide-polynucleotide expression cassettes comprise a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, in vivo.

### FIELD OF THE INVENTION

The present invention relates to a new method to assemble gRNA containing vectors to be used in CRISPR-CAS-mediated genomic transformations in a host cell.

### BACKGROUND TO THE INVENTION

Recent advances in genomics techniques and analysis methods have significantly accelerated the ability to e.g. catalog and map genetic factors associated with a diverse range of biological functions and diseases. Precise genome engineering technologies are needed to enable systematic reverse engineering of casual genetic variations by allowing selective perturbation of individual genetic elements, as well as to advance synthetic biology, biotechnological, and medical applications. Although genome-editing techniques such as designer zinc fingers, transcription activator-like effectors nucleases (TALENs), or homing meganucleases are available for producing targeted genome perturbations, there remains a need for new genome engineering technologies that are affordable, easy to set up, scalable, and amenable to targeting multiple positions within a genome. The engineering of meganucleases has been challenging for most academic researchers because the DNA recognition and cleavage functions of these enzymes are intertwined in a single domain. Robust construction of engineered zinc finger arrays has also proven to be difficult for many laboratories because of the need to account for context-dependent effects between individual finger domains in an array. There thus exists a pressing need for alternative and robust techniques for targeting of specific sequences within a host cell with a wide array of applications.

Very recently a new genome editing system, the CRISPR-Cas system, has been described (Sander et al., 2014; Hsu et al., 2014; Doudna and Charpentier, 2104). The CRISPR-Cas system does not require the generation of customized proteins to target-specific sequences but rather a single Cas enzyme that can be programmed by a guide-polynucleotide to recognize a specific polynucleotide target; in other words, the Cas enzyme can be recruited to a specific polynucleotide target in a host cell genome using said guide-polynucleotide molecule.

To enable genome precision engineering in the yeast Saccharomyces cerevisiae using the RNA-guided CRISPR/CAS9 system, the essential components CAS9 protein and the crRNA-tracrRNA fusion transcript (referred to as guide-RNA or gRNA) are introduced in the yeast cell and act together. This can be efficiently achieved by expressing both the CAS9 protein and the gRNA from one or more deoxy ribo polynucleotides. It was shown that the CAS9 protein can be expressed from a single copy vector using a centromeric episomal vector (Sikorski and Hieter, 1989) together with a 2μ vector (Christianson et al., 1992) expressing the guide-RNA and introducing donor DNA in the transformation, resulting in cells with near 100% donor DNA recombination frequency as shown by the introduction of a desired mutation (DiCarlo et al., 2013). This first study by DiCarlo and colleagues described the use of the CRISPR/CAS9 system to modify a single target in the genome within one yeast transformation round.

Expanding on the DiCarlo approach, others have described expression systems (e.g., yeast expression plasmids, use of in vivo recombination in yeast) to enable multiple modifications per transformation round, i.e., the expression of multiple gRNA sequences and donor DNA sequences (Ryan et al., 2014; Bao et al., 2015; Horwitz et al., 2015; Jakočiūnas et al., 2015; Mans et al., 2015). The gRNA expression cassettes (under control of the SNR52 promoter and SUP4 terminator as described by DiCarlo et al., 2013) are expressed from one or more high copy yeast expression vectors (i.e., bearing the sequence for the 2-μ selfish replication plasmid and a choice of dominant resistance or auxotrophic markers). For the construction of the (multi-)gRNA expression plasmids, so far two strategies are followed: either the plasmids are constructed before introduction to S. cerevisiae, for example by using an in-vitro DNA assembly method like Gibson cloning (Mans et aL, 2015), or USER cloning (Ryan et al., 2014; Jakočiūnas et al., 2015) and an intermediate cloning step in E. coli. In all these methods, which are rather laborious, full gRNA expression cassettes are used as building blocks in the cloning reaction. A second approach relies on the in vivo recombination capability of S. cerevisiae to assemble the fragments into a functional expression vector (Horwitz et al., 2015). For example, a linearized backbone vector and multiple linear gRNA expression cassettes were co-transformed directly into S. cerevisiae to generate multiple functional expression vectors by gap repair. The latter method of direct transformation of PCR-amplified or synthetic DNA fragments and the subsequent in vivo recombination by S. cerevisiae has the advantage that no intermediate amplification step, for example via E. coli is needed, shortening time and circumventing possible recombination events in E. coli.

This gRNA transformation protocol and expression system resulted in either the triple deletion of three S. cerevisiae genes (ADH5, HO, RHR2) or the introduction of single nucleotide mutations in several S. cerevisiae genes leading to easily identifiable phenotypes (e.g. SPT15 mutations and ethanol tolerance). A disadvantage of the latter method relies in the use of full expression cassettes for each guide RNA to be introduced which renders this method not very suitable for the use of standardized vector elements in the assembly. Furthermore the gap repair was dependent of 500 bp flanking sequences on each terminus of the gRNA expression cassettes. This method therefore requires the laborious construction of multiple gRNA expression cassettes comprising each approximately 1500 nucleotides. Another disadvantage relies in the efficiency of the CRISPR-Cas9 mediated genome editing which differed considerably between experiments (triple modification in one yeast transformation round leading to 64% with deletions and 9% with introduction of single nucleotide mutations). The latter might be due to the fact that not all gRNA's were equally expressed in the cell. An approach in which all gRNA are present in one single vector would be therefore preferred.

It is therefore desired to develop low-cost reliable methods for assembling of multiple gRNA-containing vectors in vivo, by using standard building blocks in the assembly.

### SUMMARY OF THE INVENTION

The present invention relates to the CRISPR-CAS system.

The present invention provides a method to produce a vector, preferably a circular vector comprising one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes;
subjecting the one or more hybrid linear polynucleotides obtained in the overlap-extension PCR and optionally one or more additional linear polynucleotides to an assembly reaction yielding a vector, preferably a circular vector comprising one or more functional guide-polynucleotide expression cassettes. Preferably the assembly reaction occurs in vivo.

The present invention further provides a method of assembling a vector, preferably a circular vector comprising one or more guide-polynucleotide expression cassettes in vivo, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
providing one or more linear polynucleotides, wherein said one or more linear polynucleotides comprise at the 5′-terminus and/or at the 3′-terminus at least one guide-polynucleotide expression cassette or a fragment thereof comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes;
optionally selecting a host cell comprising a vector, preferably a circular vector and wherein said vector, preferably said circular vector comprises one or more functional guide-polynucleotide expression cassettes;
optionally recovering the vector, preferably the circular vector.

The present invention also provides a method for producing a recombinant host cell comprising a vector, preferably a circular vector, wherein said vector, preferably said circular vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
providing one or more linear polynucleotides, wherein said one or more linear polynucleotides comprise at the 5′-terminus and/or at the 3′-terminus at least one guide-polynucleotide expression cassette or a fragment thereof comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes;
optionally selecting a host cell comprising a vector, preferably a circular vector and wherein said vector, preferably said circular vector comprises one or more functional guide-polynucleotide expression cassettes.

The present invention provides as well a method for producing a recombinant host cell comprising a vector, preferably a circular vector, wherein said vector, preferably said circular vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
performing a method of assembling a vector, preferably a circular vector comprising one or more guide-polynucleotide expression cassettes in vivo according to the present invention in a first host cell, preferably a first host cell belonging to the species S. cerevisiae, wherein said first host cell comprising the vector, preferably the circular vector, is selected and the vector, preferably the circular vector is recovered, wherein said vector, preferably said circular vector comprises one or more functional guide-polynucleotide expression cassettes,
transforming the vector, preferably the circular vector in a second host cell;
optionally isolating the second host cell comprising the vector, preferably the circular vector.

Furthermore the present invention also provides vectors, preferably circular vectors and host cell comprising a vector, preferably a circular vector obtainable with the methods according to the invention.

The present invention relates to a non-naturally occurring or engineered composition comprising a source of a CRISPR-Cas system comprising a guide-polynucleotide and a Cas protein as described herewith, wherein the guide-polynucleotide and optionally a Cas protein are present in a vector, preferably a circular vector according to the present invention.

The present invention further relates to a method of modulating expression of a polynucleotide in a cell, comprising contacting a host cell with the non-naturally occurring or engineered composition comprising a source of a CRISPR-Cas system comprising a guide-polynucleotide and a Cas protein as described herewith, wherein the guide-polynucleotide directs binding of the Cas protein at the target-polynucleotide in the host cell to form a CRISPR-Cas complex.

The present invention further relates to a method for the production of a compound of interest, comprising culturing under conditions conducive to the production of a compound of interest in a host cell according to the present invention and optionally purifying or isolating the compound of interest.

### BRIEF DESCRIPTION OF THE DRAWINGS

FIG. 1 depicts examples of typical guide-polynucleotides. Both guide-polynucleotides are guide-RNA's comprising a guide-sequence (crRNA) and a guide-polynucleotide structural component. In the upper figure, the guide-polynucleotide structural component is comprised of two separate molecules hybridized to each other; the individual molecules may be referred to as a tracr sequence and a tracr-mate sequence. In the lower figure, the guide-polynucleotide structural component is comprised of a single molecule with internal hybridization. This figure is adapted from Sander and Joung, 2014 and Mali et al., 2013.

FIG. 2 depicts the structure of bricks 1 to 4 and of the primers used in the OE-PCR reactions.

FIG. 3 depicts the structure of extended bricks 1 to 4 used in the assembly reaction in vivo.

FIG. 4 depicts how extended bricks 1 to 4 are assembled with each other in a pre-determined order in the assembly reaction in vivo.

FIG. 5 depicts a vector map of single copy (CEN/ARS) vector pCSN061 expressing CAS9 codon pair optimized for expression in S. cerevisiae. A KanMX marker is present on the vector.

FIG. 6 depicts the structure of bricks 1, 2, 3b, 4 and of the primers used in the OE-PCR reactions.

FIG. 7A depicts the structure of extended bricks 1, 2, 3b and 4 used in the assembly reaction in vivo.

FIG. 7B depicts how extended bricks 1 to 4 are assembled with each other in a pre-determined order in the assembly reaction in vivo.

FIG. 8 depicts a schematic representation of the donor DNA sequences used in the multiplex approach described in transformation Experiment 1. The donor DNA sequences (carotenoid gene expression cassettes) contain approximately 50 bp flanks sequences (INT 5′ and INT 3′), that have homology with the intended integration sites (INT1, INT2 or INT3). Upon transformation, the donor DNA sequences integrate into genomic intended integration sites. Three different promoters were used for expression of crtE, crtYB and crtI, as represented by the white and two different grey colored rectangles.

FIG. 9A depicts a schematic representation of the donor DNA, sequences used in the multiplex approach described in transformation Experiment 2. The donor DNA sequences (carotenoid gene expression cassettes) contain approximately 50 bp flanks sequences (INT 5′ and INT 3′), that have homology with the intended integration sites (INT1, INT2 or INT3). The LF sequences contain overlap with integration sites in genomic DNA and the 50 bp flank sequences plus part of a specific promoter used for expression of crtE, crtYB and crtI (indicated by the small white and two different grey colored rectangles attached to the LF sequences). The RF sequences contain overlap with integration sites in genomic DNA and the 50 bp flank sequences plus part of a specific terminator used for expression of crtE, crtYB and crtI (indicated by the small white rectangle attached to the RF sequences).

FIG. 9B depicts that upon transformation, the donor DNA sequences described in FIG. 9A integrate into genomic intended integration sites. Three different promoters were used for expression of crtE, crtYB and crtI, as represented by the white and two different grey colored rectangles.

FIG. 10 depicts a vector map of multi copy (2 micron) vector pGNR002, containing guide RNA expression cassette in which the guide sequence can be cloned/assembled by making use of the SapI sites. A NatMX (nourseothricin) resistance marker is present on the vector.

FIG. 11 depicts a graphical representation of the primers used in the OE-PCR reactions, in which vector pGRN002 was used as template, used for 1gRNA-vector assembly approach 1 and 1gRNA-vector assembly approach 2.

FIGS. 12A and 12B depict a graphical representation of PCR fragment #1 (one gRNA-vector approach 1) and PCR fragment #2 (one gRNA-vector approach 2), used in the in vivo assembly reaction in yeast. GS: 20 bp guide sequence.

FIG. 13 depicts a graphical representation of the strategy for deletion of approximately 1 or approximately 10 kb of genomic DNA around the INT1 locus using 1gRNA-vector assembly approach 1 or 1gRNA-vector assembly approach 2.

### DESCRIPTION OF THE SEQUENCE LISTING

SEQ ID NO: 1 sets out a preferred termination polynucleotide sequence in yeast.

SEQ ID NO: 2 sets out the polynucleotide sequence to be integrated in the integration locus on chromosome 14 of S. cerevisiae, comprising the Cas9 expression cassette and the natMX marker and including the 5′- and 3′-flanking regions to the integration locus.

SEQ ID NO: 3 sets out the polynucleotide sequence of Brick 1 comprising the 21 plasmid.

SEQ ID NO: 4 sets out the polynucleotide sequence of Brick 2 comprising the expression cassette of Dasher GFP, flanked upstream by the S. cerevisae TDH3 promoter, and flanked downstream by the S. cerevisiae ADH1 terminator sequence.

SEQ ID NO: 5 sets out the polynucleotide sequence of Brick 3 comprising the dominant resistance marker KanMX.

SEQ ID NO: 6 sets out the polynucleotide sequence of Brick 4 comprising the kanR gene, a bacterial selection marker, and pUC ORI.

SEQ ID NO: 7 sets out the polynucleotide sequence of forward primer gR1-HXT1-f.

SEQ ID NO: 8 sets out the polynucleotide sequence of forward primer gR2-HXT2-f.

SEQ ID NO: 9 sets out the polynucleotide sequence of forward primer gR3-HXT3-f.

SEQ ID NO: 10 sets out the polynucleotide sequence of forward primer gR4-GAL2-f.

SEQ ID NO: 11 sets out the polynucleotide sequence of reverse primer gR1-HXT1-r.

SEQ ID NO: 12 sets out the polynucleotide sequence of reverse primer gR2-HXT2-r.

SEQ ID NO: 13 sets out the polynucleotide sequence of reverse primer gR3-HXT3-r.

SEQ ID NO: 14 sets out the polynucleotide sequence of reverse primer gR4-GAL2-r.

SEQ ID NO: 15 sets out the polynucleotide sequence of extended Brick 1.

SEQ ID NO: 16 sets out the polynucleotide sequence of extended Brick 2.

SEQ ID NO: 17 sets out the polynucleotide sequence of extended Brick 3.

SEQ ID NO: 18 sets out the polynucleotide sequence of extended Brick 4.

SEQ ID NO: 19 sets out the polynucleotide sequence of the donor DNA used in the CRISPR-CAS9-mediated deletion of genes HXT5-HXT1-HXT4 in S. cerevisiae strain BIE272.

SEQ ID NO: 20 sets out the polynucleotide sequence of the donor DNA used in the CRISPR-CAS9-mediated deletion of gene HXT2 in S. cerevisiae strain BIE272.

SEQ ID NO: 21 sets out the polynucleotide sequence of the donor DNA used in the CRISPR-CAS9-mediated deletion of genes HXT3-HXT6-HXT7 in S. cerevisiae strain BIE272.

SEQ ID NO: 22 sets out the polynucleotide sequence of the donor DNA used in the CRISPR-CAS9-mediated deletion of gene GAL2 in S. cerevisiae strain BIE272.

SEQ ID NO: 23 sets out the polynucleotide sequence of CAS9 expression vector pCSN061.

SEQ ID NO: 24 sets out the polynucleotide sequence of Brick 3b (natMX-fragment).

SEQ ID NO: 25 sets out the polynucleotide sequence of forward primer g1-INT59-f.

SEQ ID NO: 26 sets out the polynucleotide sequence of forward primer g2-YPRc-f.

SEQ ID NO: 27 sets out the polynucleotide sequence of forward primer g3-INT1-f.

SEQ ID NO: 28 sets out the polynucleotide sequence of forward primer Brick4_to3-f.

SEQ ID NO: 29 sets out the polynucleotide sequence of reverse primer g1-INT59-r.

SEQ ID NO: 30 sets out the polynucleotide sequence of reverse primer g2-YPRc-r.

SEQ ID NO: 31 sets out the polynucleotide sequence of reverse primer g3-INT1-r.

SEQ ID NO: 32 sets out the polynucleotide sequence of reverse primer Brick 3_to4-r.

SEQ ID NO: 33 sets out the polynucleotide sequence of extended Brick 1 (example 4).

SEQ ID NO: 34 sets out the polynucleotide sequence of extended Brick 2 (example 4).

SEQ ID NO: 35 sets out the polynucleotide sequence of extended Brick 3b (Example 4).

SEQ ID NO: 36 sets out the polynucleotide sequence of extended Brick 4 (Example 4).

SEQ ID NO: 37 EMPTY.

SEQ ID NO: 38 sets out the polynucleotide sequence of the synthetic con5—Low p (KITDH2p)—crtE—ScTDH3t—conA expression cassette.

SEQ ID NO: 39 sets out the polynucleotide sequence of the synthetic con5—Med p (KIPGK1p)—crtE—ScTDH3t—conA expression cassette.

SEQ ID NO: 40 sets out the polynucleotide sequence of the synthetic con5—Strong p (ScFBA1p)—crtE—ScTDH3t—conA expression cassette.

SEQ ID NO: 41 sets out the polynucleotide sequence of the synthetic conA—Low p (KIYDRp)—crtYB—ScPDClt—conB expression cassette.

SEQ ID NO: 42 sets out the polynucleotide sequence of the synthetic conA—Med p (KITEF2p)—crtYB—ScPDC1t—conB expression cassette.

SEQ ID NO: 43 sets out the polynucleotide sequence of the synthetic conA—Strong p (ScTEF1p)—crtYB—ScPDClt—conB expression cassette.

SEQ ID NO: 44 sets out the polynucleotide sequence of the synthetic conB—Low p (ScPRE3p)—crtI—ScTAL1t—conC expression cassette.

SEQ ID NO: 45 sets out the polynucleotide sequence of the synthetic conB—Med p (ScACT1p)—crtI—ScTAL1t—conC expression cassette.

SEQ ID NO: 46 sets out the polynucleotide sequence of the synthetic conB—Strong p (KIENO1p)—crtI—ScTAL1t—conC expression cassette.

SEQ ID NO: 47 sets out the polynucleotide sequence of the donor expression cassette sequence with 50 bp LF and RF flanks (PCR fragment). Homology to INT1—Low p (KITDH2p)—crtE—ScTDH3t—Homology to INT1.

SEQ ID NO: 48 sets out the polynucleotide sequence of the donor expression cassette sequence with 50 bp LF and RF flanks (PCR fragment). Homology to INT1—Med p (KIPGK1p)—crtE—ScTDH3t—Homology to INT1.

SEQ ID NO: 49 sets out the polynucleotide sequence of the donor expression cassette sequence with 50 bp LF and RF flanks (PCR fragment). Homology to INT1—Strong p (ScFBA1p)—crtE—ScTDH3t-Homology to INT1.

SEQ ID NO: 50 sets out the polynucleotide sequence of the donor expression cassette sequence with 50 bp LF and RF flanks (PCR fragment). Homology to INT2—Low p (KIYDR1p)—crtYB—ScPDC1t—Homology to INT2.

SEQ ID NO: 51 sets out the polynucleotide sequence of the donor expression cassette sequence with 50 bp LF and RF flanks (PCR fragment). Homology to INT2—Med p (KITEF2p)—crtYB—ScPDC1t—Homology to INT2.

SEQ ID NO: 52 sets out the polynucleotide sequence of the donor expression cassette sequence with 50 bp LF and RF flanks (PCR fragment). Homology to INT2—Strong p (ScTEF1p)—crtYB—ScPDC1t—Homology to INT2.

SEQ ID NO: 53 sets out the polynucleotide sequence of the donor expression cassette sequence with 50 bp LF and RF flanks (PCR fragment). Homology to INT3—Low p (ScPRE3p)—crtI—ScTAL1 t—Homology to INT3.

SEQ ID NO: 54 sets out the polynucleotide sequence of the donor expression cassette sequence with 50 bp LF and RF flanks (PCR fragment). Homology to INT3—Med p (ScACT1p)—crtI—ScTAL1 t—Homology to INT3.

SEQ ID NO: 55 sets out the polynucleotide sequence of the donor expression cassette sequence with 50 bp LF and RF flanks (PCR fragment). Homology to INT3—Strong p (KIENO1p)—crtI—ScTAL1 t—Homology to INT3.

SEQ ID NO: 56 sets out the polynucleotide sequence of the forward (FW) primer to obtain SEQ ID NO: 47.

SEQ ID NO: 57 sets out the polynucleotide sequence of the reverse (REV) primer to obtain SEQ ID NO: 47, 48, 49.

SEQ ID NO: 58 sets out the polynucleotide sequence of the FW primer to obtain SEQ ID NO: 48.

SEQ ID NO: 59 sets out the polynucleotide sequence of the FW primer to obtain SEQ ID NO: 49.

SEQ ID NO: 60 sets out the polynucleotide sequence of the FW primer to obtain SEQ ID NO: 50.

SEQ ID NO: 61 sets out the polynucleotide sequence of the REV primer to obtain SEQ ID NO: 50, 51, 52.

SEQ ID NO: 62 sets out the polynucleotide sequence of the FW primer to obtain SEQ ID NO: 51.

SEQ ID NO: 63 sets out the polynucleotide sequence of the FW primer to obtain SEQ ID NO: 52.

SEQ ID NO: 64 sets out the polynucleotide sequence of the FW primer to obtain SEQ ID NO: 53.

SEQ ID NO: 65 sets out the polynucleotide sequence of the REV primer to obtain SEQ ID NO: 53, 54, 55.

SEQ ID NO: 66 sets out the polynucleotide sequence of the FW primer to obtain SEQ ID NO: 54.

SEQ ID NO: 67 sets out the polynucleotide sequence of the FW primer to obtain SEQ ID NO: 55.

SEQ ID NO: 68 sets out the polynucleotide sequence of the Right Flank (RF) sequence with overlap ScTDH3t—INT1 3′ (part of donor)—INT1 3′ genomic DNA. Homology with INT1 3′ genomic DNA 524 bp.

SEQ ID NO: 69 sets out the polynucleotide sequence of the Left Flank (LF) sequence with overlap KIYDR2p—INT2 5′ (part of donor)—INT2 5′ genomic DNA. Homology with INT2 5′ genomic DNA 322 bp.

SEQ ID NO: 70 sets out the polynucleotide sequence of the LF sequence with overlap KITEF2p—INT2 5′ (part of donor)—INT2 5′ genomic DNA. Homology with INT2 5′ genomic DNA 322 bp.

SEQ ID NO: 71 sets out the polynucleotide sequence of the LF sequence with overlap ScTEF1p—INT2 5′ (part of donor)—INT2 5′ genomic DNA. Homology with INT2 5′ genomic DNA 322 bp.

SEQ ID NO: 72 sets out the polynucleotide sequence of the RF sequence with overlap ScPDC1t—INT2 3′ (part of donor)—INT2 3′ genomic DNA. Homology with INT2 3′ genomic DNA 524 bp.

SEQ ID NO: 73 sets out the polynucleotide sequence of the LF sequence with overlap ScPRE3p—INT3 5′ (part of donor)—INT3 5′ genomic DNA. Homology with INT3 5′ genomic DNA 602 bp.

SEQ ID NO: 74 sets out the polynucleotide sequence of the LF sequence with overlap ScACT1p—INT3 5′ (part of donor)—INT3 5′ genomic DNA. Homology with INT3 5′ genomic DNA 602 bp.

SEQ ID NO: 75 sets out the polynucleotide sequence of the LF sequence with overlap KIENO1p—INT3 5′ (part of donor)—INT3 5′ genomic DNA. Homology with INT3 5′ genomic DNA 602 bp.

SEQ ID NO: 76 sets out the polynucleotide sequence of the RF sequence with overlap ScTAL1t—INT3 3′ (part of donor)—INT3 3′ genomic DNA. Homology with INT3 3′ genomic DNA 624 bp.

SEQ ID NO: 77 sets out the polynucleotide sequence of the FW primer to obtain SEQ ID NO: 68.

SEQ ID NO: 78 sets out the polynucleotide sequence of the REV primer to obtain SEQ ID NO: 68.

SEQ ID NO: 79 sets out the polynucleotide sequence of the FW primer to obtain SEQ ID NO: 69, 70,71.

SEQ ID NO: 80 sets out the polynucleotide sequence of the REV primer to obtain SEQ ID NO: 69.

SEQ ID NO: 81 sets out the polynucleotide sequence of the REV primer to obtain SEQ ID NO: 70.

SEQ ID NO: 82 sets out the polynucleotide sequence of the REV primer to obtain SEQ ID NO: 71.

SEQ ID NO: 83 sets out the polynucleotide sequence of the FW primer to obtain SEQ ID NO: 72.

SEQ ID NO: 84 sets out the polynucleotide sequence of the REV primer to obtain SEQ ID NO: 72.

SEQ ID NO: 85 sets out the polynucleotide sequence of the FW primer to obtain SEQ ID NO: 73, 74, 75.

SEQ ID NO: 86 sets out the polynucleotide sequence of the REV primer to obtain SEQ ID NO: 73.

SEQ ID NO: 87 sets out the polynucleotide sequence of the REV primer to obtain SEQ ID NO: 74.

SEQ ID NO: 88 sets out the polynucleotide sequence of the REV primer to obtain SEQ ID NO: 75.

SEQ ID NO: 89 sets out the polynucleotide sequence of the FW primer to obtain SEQ ID NO: 76.

SEQ ID NO: 90 sets out the polynucleotide sequence of the REV primer to obtain SEQ ID NO: 76.

SEQ ID NO: 91 sets out the polynucleotide sequence of expression vector pRN1120.

SEQ ID NO: 92 sets out the polynucleotide sequence of FW primer to remove SapI restriction site in pRN1120.

SEQ ID NO: 93 sets out the polynucleotide sequence of REV primer to remove SapI restriction site in pRN1120.

SEQ ID NO: 94 sets out the polynucleotide sequence of gBlock allowing direct SapI cloning of the guide sequence, part of vector pGRN002.

SEQ ID NO: 95 sets out the polynucleotide sequence of expression vector pGRN002.

SEQ ID NO: 96 sets out the polynucleotide sequence of FW primer approach 1: 30 bp on either side.

SEQ ID NO: 97 sets out the polynucleotide sequence of REV primer approach 1: 30 bp on either side.

SEQ ID NO: 98 sets out the polynucleotide sequence of PCR fragment approach 1: 30 bp on either side. The fragment was transformed to yeast allowing reconstitution of a circular vector by in vivo recombination.

SEQ ID NO: 99 sets out the polynucleotide sequence of FW primer approach 1: 50 bp tail on one side.

SEQ ID NO: 100 sets out the polynucleotide sequence of REV primer approach 1: 50 bp tail on one side.

SEQ ID NO: 101 sets out the polynucleotide sequence of PCR fragment approach 2: 50 bp tail on one side. The fragment was transformed to yeast allowing reconstitution of a circular vector by in vivo recombination.

SEQ ID NO: 102 sets out the polynucleotide sequence of FW primer to obtain the 5′ flank A—connector 5 sequence to obtain approximately 1 kB deletion of genomic DNA.

SEQ ID NO: 103 sets out the polynucleotide sequence of REV primer to obtain the 5′ flank A and B—connector 5 sequence to obtain approximately 1 kB deletion of genomic DNA.

SEQ ID NO: 104 sets out the polynucleotide sequence of fragment A (5′ flank A—connector 5) to obtain approximately 1 kB deletion of genomic DNA.

SEQ ID NO: 105 sets out the polynucleotide sequence of FW primer to obtain the 5′ flank B—connector 5 sequence to obtain approximately 10 kB deletion of genomic DNA.

SEQ ID NO: 106 sets out the polynucleotide sequence of fragment B (5′ flank B—connector 5) to obtain approximately 10 kB deletion of genomic DNA.

SEQ ID NO: 107 sets out the polynucleotide sequence of FW primer to obtain Connector 5—TDH3p—GFP—ENO1t—Connector 3 PCR fragment.

SEQ ID NO: 108 sets out the polynucleotide sequence of REV primer to obtain Connector 5—TDH3p—GFP—ENO1t—Connector 3 PCR fragment.

SEQ ID NO: 109 sets out the polynucleotide sequence of fragment C, Connector 5—TDH3p—GFP—ENO1t—Connector 3 synthetic cassette.

SEQ ID NO: 110 sets out the polynucleotide sequence of FW primer to obtain the connector 3-3′ flank A sequence to obtain approximately 1 kB deletion of genomic DNA.

SEQ ID NO: 111 sets out the polynucleotide sequence of REV primer to obtain the connector 3-3′ flank A and B sequence to obtain approximately 1 kB deletion of genomic DNA.

SEQ ID NO: 112 sets out the polynucleotide sequence of fragment D (connector 3-3′ flank A) to obtain approximately 1 kB deletion of genomic DNA.

SEQ ID NO: 113 sets out the polynucleotide sequence of FW primer to obtain the connector 3-3′ flank B sequence to obtain approximately 10 kB deletion of genomic DNA.

SEQ ID NO: 114 sets out the polynucleotide sequence of REV primer to obtain the connector 3-3′ flank B sequence to obtain approximately 10 kB deletion of genomic DNA.

SEQ ID NO: 115 sets out the polynucleotide sequence of fragment E (connector 3-3′ flank B) to obtain approximately 10 kB deletion of genomic DNA.

SEQ ID NO: 116 sets out the polynucleotide sequence of the first 15 polynucleotides (nucleotide 1-15) in the forward primers according to SEQ ID NO: 7 to 10.

SEQ ID NO: 117 sets out the polynucleotide sequence of the last 25 polynucleotides (nucleotide 36-60) in the forward primers according to SEQ ID NO: 7 to 10.

SEQ ID NO: 118 sets out the polynucleotide sequence of the first 15 polynucleotides (nucleotide 1-15) in the reverse primers according to SEQ ID NO: 11 to 14.

SEQ ID NO: 119 sets out the polynucleotide sequence of the last 25 polynucleotides (nucleotide 36-60) in the reverse primers according to SEQ ID NO: 11 to 14.

### DETAILED DESCRIPTION OF THE INVENTION

We have surprisingly found that vectors comprising multiple gRNA-expression cassettes can be efficiently produced by applying re-usable, standardized vector elements together with overlap-extension-PCR (OE-PCR) to introduce the variable guide sequences. Due to the design of the multi-fragment assembly according to the invention, functional and multi-gRNA cassettes can be assembled straightforward at low cost. The method described herewith provides the flexibility to work with a set of standardized vector elements that can be re-used in a flexible way, e.g., a set of standardized marker cassettes, Origing of Replication cassettes, fluorescent/chromogenic cassettes, promoters, terminators etc.

In a first aspect the invention relates to a method to produce a vector, preferably a circular vector comprising one or more guide-polynucleotide expression cassettes, wherein said one or more guide-polynucleotide expression cassettes comprise a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes;
subjecting the one or more hybrid linear polynucleotides obtained in the overlap-extension PCR and optionally one or more additional linear polynucleotides to an assembly reaction yielding a vector, preferably a circular vector comprising one or more functional guide-polynucleotide expression cassettes,

wherein the assembly reaction occurs in vivo.

A “guide-polynucleotide expression cassette” is herewith defined as an expression cassette comprising a polynucleotide encoding a guide-polynucleotide operably linked to one or more control sequences which direct the expression of said guide-polynucleotide in a suitable host cell. In the context of the present invention a “guide-polynucleotide” comprises a guide-sequence that essentially is the reverse complement of a target-polynucleotide in a host cell and the guide-polynucleotide can direct binding of a Cas protein at a target-polynucleotide in a host cell to form a CRISPR-Cas complex.

The term “CRISPR-Cas complex” refers in the context of all embodiments of the present invention to a complex comprising a guide-polynucleotide hybridized to a target-polynucleotide and complexed with a Cas protein. In the most straightforward form, where a non-mutated Cas protein is used such as but not limited to the Cas9 protein of Streptococcus pyogenes, the formation of the CRISPR-Cas complex results in cleavage of one or both polynucleotide strands in or near (e.g. within 1, 2, 3, 4, 5, 6, 7, 8, 9, 10, 20, 50, or more base pairs from) the target-polynucleotide. Typically, a target-polynucleotide according to the present invention (defined below herein) is associated with a PAM sequence (defined below herein) and the PAM sequence is preferably immediately downstream (3′) of the target-polynucleotide; the formation of the CRISPR-Cas complex typically results in cleavage of one or both polynucleotide strands 3 base pairs upstream (5′) of the PAM sequence.

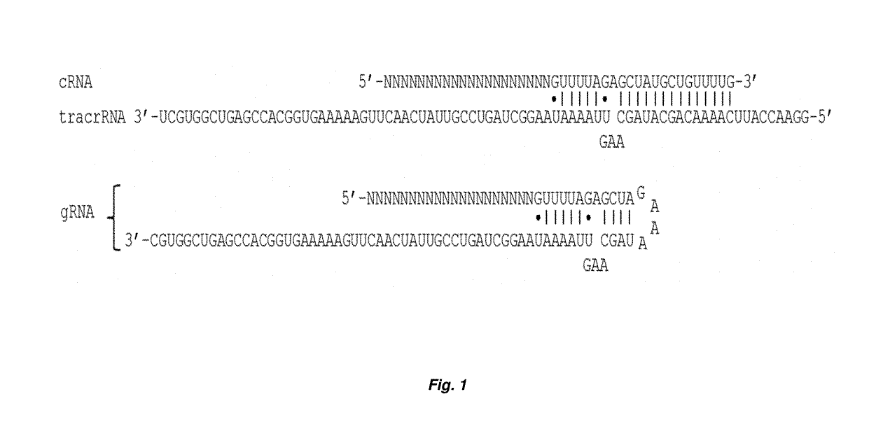

A “guide-polynucleotide” as intended herewith is a polynucleotide comprising at least a guide-sequence that is able to hybridize with the target-polynucleotide, e.g. a target polynucleotide in the genome of a host cell, and is able to direct sequence-specific binding of the CRISPR-Cas system to the target-polynucleotide to form a CRISPR-Cas complex. In one embodiment the guide-polynucleotide is a gRNA. In order to enable formation of an active CRISPR-Cas complex, the guide-polynucleotide preferably also comprises a sequence that has a specific secondary structure and allows binding of the Cas protein to the guide-polynucleotide. Such sequence is known in the art as tracrRNA, tracr sequence, tracr scaffold or guide-polynucleotide structural component, these terms are used interchangeably herein; wherein the tracr is the abbreviation for transactivating CRISPR; tracrRNA thus means transactivating CRISPR RNA. The tracrRNA in the original CRISPR-Cas system is the endogenous bacterial RNA that links the crRNA (guide-sequence) to the Cas nuclease, being able to bind any crRNA. A guide-polynucleotide structural component may be comprised of a single polynucleotide molecule or may be comprised of two or more molecules hybridized to each other; such hybridizing components of a guide-polynucleotide structural component may be referred to as a tracr sequence and a tracr-mate sequence. Accordingly, the guide-polynucleotide preferably also comprises a tracr sequence and/or a tracr-mate sequence. The guide-polynucleotide is a polynucleotide according to the general definition of a polynucleotide set out here below; a preferred guide-polynucleotide comprises ribonucleotides, a more preferred guide-polynucleotide is a RNA (guide-RNA or gRNA). Two examples of typical guide-polynucleotide structures are depicted in FIG. 1.

In the context of the present invention, a “guide-sequence” is referred to as essentially the reverse complement of a target-sequence or of a target-polynucleotide, e.g. a target-sequence or target-polynucleotide present in the genome of a host cell, if the subject sequence is able to hybridize with the target-sequence or target-polynucleotide, preferably under physiological conditions as in a host cell. The degree of complementarity between a guide-sequence and its corresponding target-sequence, when optimally aligned using a suitable alignment algorithm, is preferably higher than 50%, 60%, 75%, 80%, 85%, 90%, 95%, 97.5%, 99% sequence identity.

Optimal alignment may be determined using any suitable algorithm for aligning sequences, preferably an algorithm as defined herein under “Sequence identity”. When the target-polynucleotide is a double stranded polynucleotide, the subject sequence, such as a guide-sequence, may be able to hybridize with either strand of the target-polynucleotide e.g. a coding strand or a non-coding strand. Preferably, a guide-sequence according to the present invention targets a target-sequence that is unique in the target. Preferably, a guide-sequence according to the present invention has 100% sequence identity with the 8, 9, 10, 11, 12, 13, 14, 15, 16, 17, 18, 19 or 20, more preferably 8, 9, 10, 11 or 12 nucleotides in the target-polynucleotide immediately adjacent to a PAM sequence. A guide-sequence according to the present invention preferably is 8-30, more preferably 10-30, more preferably 15-30, more preferably 17-27, more preferably 17-20, more preferably 17, 18, 19, 20, 21, 22, 23, 24, 25, 26, or 27 nucleotides in length. The ability of a guide-sequence to direct sequence-specific binding of a CRISPR-Cas system to a target-sequence to form a CRISPR-Cas complex may be assessed by any suitable assay. For example, the components of a CRISPR system sufficient to form a CRISPR-Cas complex, including the guide-sequence to be tested, may be provided to a host cell having the corresponding target-sequence, such, as by transfection with vectors encoding the components of the CRISPR-Cas system, followed by an assessment of preferential cleavage within the target-sequence, such as by the Surveyor assay (Surveyor® Mutation Detection Kits distributed by Integrated DNA Technologies, Leuven, Belgium) or another sequence analysis assay such as sequencing. Cleavage of a target-polynucleotide may be evaluated in a test tube by providing the target-polynucleotide, components of a CRISPR-Cas system, including the guide-sequence to be tested and a control guide-sequence different from the test guide-sequence, and comparing binding or rate of cleavage at the target-sequence between the test and control guide-sequence reactions. Other assays are possible, and are known to a person skilled in the art. A guide-polynucleotide structural component is believed to be necessary for formation of an active CRISPR-Cas complex. The guide-polynucleotide structural component is believed not necessarily to be operably linked to the guide-sequence; however, a guide-polynucleotide structural component may be operably linked to a guide-sequence within a guide-polynucleotide. A guide-polynucleotide structural component according to the present invention, which may comprise or consist of all or a portion of a wild-type guide-polynucleotide structural component (e.g. about or more than about 20, 26, 32, 45, 48, 54, 63, 67, 85, or more nucleotides of a wild-type tracr-sequence) forms part of a CRISPR-Cas complex; e.g. by hybridization of at least a portion of a tracr-sequence according to the present invention to all or a portion of a tracr-mate sequence according to the present invention and preferably operably linked to a guide-sequence according to the present invention. A tracr-sequence according to the present invention has sufficient complementarity to a tracr-mate sequence according to the present invention to hybridize, preferably under physiological condition as in a host cell, and facilitate formation of a CRISPR-Cas complex. As with the target-sequence according to the present invention, it is believed that complete complementarity is not needed, provided there is sufficient complementarity to be functional. Preferably, the tracr-sequence according to the present invention has at least 50%, 60%, 70%, 80%, 90%, 95% or 99% sequence identity along the length of the tracr-mate sequence according to the present invention when optimally aligned. Optimal alignment may be determined using any suitable algorithm for aligning sequences, preferably an algorithm as defined herein under “Sequence identity”. In general, a tracr mate sequence according to the present invention includes any sequence that has sufficient complementarity with a tracr sequence according to the present invention to promote formation of a CRISPR-Cas complex at a target-sequence, wherein the CRISPR-Cas complex comprises the tracr mate sequence according to the present invention hybridized to the tracr sequence according to the present invention. The degree of complementarity of the tracr sequence according to the present invention and the tracr mate sequence according to the present invention is preferably defined with respect to optimal alignment of the tracr mate sequence and tracr sequence along the length of the shorter of the two sequences. Optimal alignment may be determined using any suitable algorithm for aligning sequences, preferably an algorithm as defined herein under “Sequence identity”. Preferably, with respect to a tracr mate sequence according to the present invention and a tracr sequence according to the present invention, secondary structures are taken into account, such as self-complementarity within either the tracr sequence or tracr mate sequence. Preferably, the degree of complementarity between the tracr sequence according to the present invention and tracr mate sequence according to the present invention along the length of the shorter of the two sequences when optimally aligned is higher than 50%, 60%, 75%, 80%, 85%, 90%, 95%, 97.5%, 99% sequence identity. Preferably, the tracr mate sequence according to the present invention is 5, 6, 7, 8, 9, 10, 11, 12, 13, 14, 15, 16, 17, 18, 19, 20, 25, 30, 40, 50, or more nucleotides in length. Preferably, the tracr sequence according to the present invention is 5, 6, 7, 8, 9, 10, 11, 12, 13, 14, 15, 16, 17, 18, 19, 20, 25, 30, 40, 50, or more nucleotides in length. Preferably, the tracr sequence according to the present invention and tracr mate sequence, i.e. the guide-polynucleotide structural component according to the present invention are comprised within a single transcript, such that hybridization between the two produces a hybridization complex comprising a secondary structure, such as a hairpin. Such hybridization complex may also be formed when the tracr sequence and the tracr mate sequence are not comprised in a single transcript. Preferred loop forming sequences in a tracr sequence according to the present invention and/or a tracr mate sequence according to the present invention and/or guide-polynucleotide structural component according to the present invention for formation of hairpin structures are four nucleotides in length, and most preferably have the sequence GAAA; longer or shorter loop sequences may be used, as may alternative sequences. The loop sequences preferably include a nucleotide triplet (for example, AAA), and an additional nucleotide (for example C or G). Examples of loop forming sequences include CAAA and AAAG. Preferably, a tracr sequence according to the present invention and/or tracr mate sequence according to the present invention or hybridization complex thereof and/or guide-polynucleotide structural component according to the present invention comprise or is able to form at least two or more hairpins. More preferably, a tracr sequence according to the present invention and/or tracr mate sequence according to the present invention or hybridization complex thereof and/or guide-polynucleotide structural component according to the present invention comprises or is able to form two, three, four or five hairpins. Preferably, a tracr sequence according to the present invention and/or tracr mate sequence according to the present invention or hybridization complex thereof and/or guide-polynucleotide structural component according to the present invention comprises or is able to form at most five hairpins. Preferably, the single transcript of a tracr sequence according to the present invention and a tracr-mate sequence according to the present invention or hybridization complex of a tracr sequence according to the present invention and a tracr mate sequence according to the present invention and/or guide-polynucleotide structural component according to the present invention further comprises a transcription termination sequence; preferably this is a polyT sequence, for example six T nucleotides or, preferred for yeast, TTTTTTTGTTTTTTATGTCT (SEQ ID NO: 1). As said, guide-polynucleotide structural components are known to the person skilled in the art; background information can e.g. be found in Gaj et al, 2013.

In the context of all embodiments according to the present invention, the term “target-polynucleotide” refers to a target-sequence as defined herewith to which a guide-sequence as defined herewith is designed to have complementarity, where hybridization between a target-sequence as defined herewith and a guide-sequence as defined herewith promotes the formation of a CRISPR-Cas complex. Full complementarity is not necessarily required, provided there is sufficient complementarity to cause hybridization and promote formation of a CRISPR-Cas complex. Preferably, a guide-sequence as herewith defined targets a target-sequence that is unique in the target. Preferably, a guide-sequence has 100% sequence identity with the 8, 9, 10, 11, 12, 13, 14, 15, 16, 17, 18, 19 or 20, more preferably 8, 9, 10, 11 or 12 nucleotides in the target-polynucleotide immediately adjacent to a PAM sequence. A target-polynucleotide as defined herewith may comprise any polynucleotide, such as DNA or RNA polynucleotides and may be single or double stranded. When the target-polynucleotide is a double strand polynucleotide, a guide-sequence as defined herewith, may be able to hybridize with either strand of the target-polynucleotide e.g. a coding strand or a non-coding strand.

A target-polynucleotide as defined herewith may be located in the nucleus or cytoplasm of a cell. A target-polynucleotide as defined herewith may be located in an organelle of a host cell, for example in a mitochondrion or chloroplast. A target-polynucleotide as defined herewith may be comprised in a genome, may be comprised in a chromosome or may be extra-chromosomal, may be comprised in an artificial chromosome such a Yeast Artificial Chromosome (YAC), may be present in any chromosomal entity or extra-chromosomal entity such as an autosomal replicating entity such as an episomal plasmid or vector. A target-polynucleotide as defined herewith may be native or foreign to the host cell. A target-polynucleotide as defined herewith is preferably associated with a protospacer adjacent motif (PAM), which is a short polynucleotide recognized by the CRISPR-Cas complex. Preferably, the target-polynucleotide and PAM are linked wherein the PAM is preferably immediately downstream (3′) of the target-polynucleotide. The exact sequence and length of the PAM may vary, e.g. different Cas proteins may require different PAM's. A preferred PAM according to the present invention is a polynucleotide of 2 to 8 nucleotides in length. A preferred PAM is selected from the group consisting of 5′-XGG-3′, 5′-XGGXG-3′, 5′-XXAGAAW-3′, 5′-XXXXGATT-3′, 5′-XXAGAA-3′, 5′-XAAAAC-3′, wherein X can be any nucleotide or analog thereof, preferably any nucleotide; and W is A or T. A more preferred PAM is 5′-XGG-3′. The PAM is preferably matched with the Cas protein. The most widely used CAS/CRISPR system is derived from S. pyogenes and the matching PAM sequence 5′-XGG-3′ is located immediately downstream (3′) of the target-sequence. A preferred PAM for a Neisseria meningitidis Cas protein is 5′-XXXXGATT-3′; a preferred PAM for a Streptococcus thermophilus Cas protein is 5′-XXAGAA-3′; a preferred PAM for a Treponema denticola is 5′-XAAAAC-3′. A preferred PAM matches the Cas protein used. A Cas protein according to the present invention may be engineered to match a different PAM than the native PAM matching the wild-type Cas protein. As such, the CRISPR-Cas system according to the present invention may be used for customized specific targeting.

Preferred genomes are those of Aspergillus, Penicillium, Rasamsonia, Trichoderma, Yarrowia, Saccharomyces, Kluyveromyces, Mortierella, Bacillus, Escherichia, Pichia and Issatchenkia, respectively. Non-limiting examples of genomes of said microorganisms can be accessed through their Genbank accession IDs numbers at for example (http://www.ncbi.nlm.nih.cov/) as follows: A. niger ATCC 1015 NCBI accession number ACJE00000000, A. niger CBS 513.88 genome sequence is accessible through EMBL accession numbers AM269948 to AM270415; Aspergillus oryzae RIB40 BA000049 to BA000056; Penicillium chrysogenum Wisconsin 54-1255 complete genome, accession numbers AM920416 to AM920464; Kluyveromyces lactis CBS2359 NRRL Y-1140 accession nos AJ229366 to AJ230023; Kluyveromyces lactis NRRL Y-1140 Accesion ID NC_006037 to NC_006042; Saccharomyces cerevisiae CEN.PK113-7D Accesion ID CM001522 to CM001537; Saccharomyces cerevisiae S288c Accesion ID NC_001133 to NC_001148; Rasamsonia Emersonii CBS 393.64 Accesion ID LASV01000001 to LASV01000862; Yarrowia lipolitica PO1f Accession ID CM002778 to CM002783; Yarrowia lipolitica CLIB122 Accession ID CR382127 to CR382132; Bacillus subtilis subsp. subtilis str. 168 Accession ID AL009126; Escherichia coli str. K-12 substr. MG1655 Accession Id U00096.3; Pichia pastoris Accession Id FN392319-FN392325, Pichia kudriavzevii M12 (also known as Issatchenkia orientalis), accession number: GenBank: ALNQ00000000.1, Trichoderma reesei QM6a GL985056-GL985132.

Unknown or ambiguous nucleotides in a genome (such as a nucleotide depicted with “n”) are preferably excluded as polynucleotide sequence target.

The terms “CRISPR system”, “CRISPR-Cas system” and “CRISPR enzyme system” are used interchangeably herein and refer in the context of all embodiments of the present invention to a collection of elements required to form, together with a target-polynucleotide, a CRISPR-Cas complex; these elements comprise but are not limited to a Cas protein and a guide-polynucleotide.

The terms “polynucleotide”, “(poly)nucleotide sequence” and “nucleic acid” are used interchangeably herein and refer in the context of all embodiments of the present invention to a polymeric form of nucleotides of any length, either deoxyribonucleotides or ribonucleotides, or mixes or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, oligonucleotides and primers. A polynucleotide may comprise one or more modified nucleotides, such as a methylated nucleotide and a nucleotide analogue or nucleotide equivalent wherein a nucleotide analogue or equivalent is defined as a residue having a modified base, and/or a modified backbone, and/or a non-natural internucleoside linkage, or a combination of these modifications.

Preferred nucleotide analogues and equivalents are described in the section “General definitions”. As desired, modifications to the nucleotide structure may be introduced before or after assembly of the polynucleotide. A polynucleotide may be further modified after polymerization, such as by conjugation with a labeling compound. A polynucleotide may be single stranded or double stranded. When referring to polynucleotide coding/encoding for another polynucleotide, generally the term polynucleotide will include in its meaning the double strand polynucleotide, even though reference will only be made to one of the two strands (i.e. reference will be made to the coding strand).

The method to produce a vector, preferably a circular vector according to the first aspect comprises in a first step:
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette.

The one or more polynucleotides may be a polynucleotide as defined herein above. Each of said one or more polynucleotides comprises a polynucleotide sequence to be extended. In the context of the present invention a polynucleotide sequence to be extended is a polynucleotide sequence comprised in each of said one or more polynucleotides which is to be extended by overlap-extension PCR reaction in the second step of the method to produce a vector, preferably a circular vector according to the invention and which become spliced/hybridized with the nucleotide sequence of the primers used in the reaction. The resulting extended polynucleotide sequence comprises the polynucleotide sequence to be extended which is spliced/hybridized with the nucleotide sequence of the primers used in the reaction. Said extended polynucleotide sequence is herewith indicated as hybrid linear polynucleotide.

The polynucleotide sequence to be extended comprised in each of said one or more polynucleotides which undergoes the overlap-extension PCR reaction may be a sub-sequence of said polynucleotide or it may comprise the whole polynucleotide sequence. Therefore in one embodiment the polynucleotide which undergoes the overlap-extension PCR reaction consists of the polynucleotide sequence to be extended.

The polynucleotide sequence to be extended comprises at the 5′-end and/or at the 3′-end to be extended a fragment of a guide-polynucleotide expression cassette.

A “guide-polynucleotide expression cassette”, as defined herein before, is a polynucleotide encoding a guide-polynucleotide operably linked to one or more control sequences which direct the expression of said guide-polynucleotide in a host cell.

The guide-polynucleotide expression cassette contains all the control sequences required for expression of the guide-polynucleotide in a host cell, wherein said control sequences are operably linked to said guide-polynucleotide. Typically a guide-polynucleotide expression cassette will comprise at least a promoter operably linked to the coding sequence of the guide-polynucleotide to be expressed in a host cell and a terminator sequence.

The term “operably linked” as used herein refers to two or more nucleic acid sequence elements that are physically linked and are in a functional relationship with each other. For instance, a control sequence, e.g. a promoter, is operably linked to a DNA coding sequence if the promoter is able to initiate or regulate the transcription or expression of a coding sequence, i.e. in a configuration in which the control sequence is appropriately placed at a position relative to the coding sequence such that the control sequence directs the production of an RNA, e.g. a mRNA or a gRNA and optionally of a polypeptide translated from said mRNA, in which case the coding sequence should be understood as being “under the control of” the promoter. Generally, when two nucleic acid sequences are operably linked, they will be in the same orientation and usually also in the same reading frame. They usually will be essentially contiguous, although this may not be required.

The term “control sequences” is defined herein to include all components, which are necessary or advantageous for the expression of RNA and/or a polypeptide (in case the control sequences are operably linked to a polynucleotide coding a compound of interest or involved in the production of a compound of interest) either in vitro or in a host cell. Such control sequences include, but are not limited to, a leader, Shine-Delgarno sequence (also indicated as Ribosome Binding Site), a polyadenylation sequence, a pro-peptide sequence, a pre-pro-peptide sequence, a promoter, a signal sequence, and a transcription terminator.

The control sequences may be provided with linkers for the purpose of introducing specific restriction sites facilitating ligation of the control sequences with the coding region of the guide polynucleotide.

The control sequence may be an appropriate promoter sequence (promoter).

The control sequence may also be a suitable transcription terminator (terminator) sequence, a sequence recognized by a host cell to terminate transcription. The terminator sequence is operably linked to the 3′-terminus of the nucleic acid sequence encoding the guide-polynucleotide or encoding a compound of interest. Any terminator, which is functional in the cell, may be used in the present invention. The person skilled in the art knows which types of terminators can be used in the host cell as described herein.

The control sequence may also be a 5′-untranslated sequence (also known as leader sequence), a non-translated region of a mRNA which is important for translation by the host cell. The translation initiation sequence or 5′-untranslated sequence is operably linked to the 5′-terminus of the coding sequence encoding a compound of interest or a compound related to the compound of interest as defined elsewhere herewith. Any leader sequence, which is functional in the cell, may be used in the present invention.

The control sequence may also be a polyadenylation sequence, a sequence which is operably linked to the 3′-terminus of the nucleic acid sequence and which, when transcribed, is recognized by the host cell (mutated or parent) as a signal to add polyadenosine residues to transcribed mRNA. Any polyadenylation sequence, which is functional in the cell, may be used in the present invention.

The term “promoter” is defined herein as a DNA sequence that binds RNA polymerase and directs the polymerase to the correct downstream transcriptional start site of a nucleic acid sequence encoding a biological compound, e.g. a compound of interest or the guide-polynucleotide, to initiate transcription. RNA polymerase effectively catalyzes the assembly of guide RNA or of messenger RNA complementary to the appropriate DNA strand of a coding region. The term “promoter” will also be understood to include the 5′-non-coding region (between promoter and translation start) for translation after transcription into mRNA (when the promoter is operably linked to a polynucleotide coding a compound of interest or coding a compound involved in the synthesis of a compound of interest), cis-acting transcription control elements such as enhancers, and other nucleotide sequences capable of interacting with transcription factors.

In the context of the present invention a “fragment of a guide-polynucleotide expression cassette” is a polynucleotide which comprises only a part of a (functional) guide-polynucleotide expression cassette. In one embodiment the fragment of a guide-polynucleotide expression cassette comprises a fragment including the 5′-terminus of the guide-polynucleotide expression cassette. In another embodiment the fragment of a guide-polynucleotide expression cassette comprises a fragment including the 3′-terminus of the guide-polynucleotide expression cassette. In one embodiment the polynucleotide sequence to be extended comprises at the 5′-end a fragment of a guide polynucleotide expression cassette comprising the 3′-terminus of said expression cassette. In another embodiment the polynucleotide sequence to be extended comprises at the 3′-end a fragment of a guide polynucleotide expression cassette comprising the 5′-terminus of a guide polynucleotide expression cassette. In one embodiment a fragment including the 5′-terminus of the guide-polynucleotide expression cassette comprises a control sequence of the guide-polynucleotide expression cassette, more preferably comprises a promoter. In another embodiment a fragment including the 3′-terminus of a guide polynucleotide expression cassette comprises a guide-polynucleotide structural element and a control sequence, more preferably comprises a guide-polynucleotide structural element and a terminator. In a preferred embodiment the fragment of a guide-polynucleotide expression cassette at the 5′-end and/or at the 3′-end of the polynucleotide sequence to be extended, does not comprise a polynucleotide coding for a guide sequence. This latter embodiment has the advantage of allowing use of standardized vector elements which together with overlap-extension-PCR (OE-PCR) allow introduction of the variable guide sequences. Therefore functional and multi-gRNA cassettes can be assembled straightforward at low cost. In a second step, the method to produce a vector, preferably a circular vector comprising one or more guide-polynucleotide expression cassettes according to the first aspect comprises:
performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

Throughout the invention the vector comprising one or more guide-polynucleotide expression cassettes, assembled in vivo according to the methods of the invention is preferably an autonomously replicating circular vector. In alternative said vector comprising one or more guide polynucleotide expression cassettes may be an autonomously replicating linear vectors. Autonomously replicanting linear vectors are known to those skilled in the art. (Finbarr Hayes (2003); Takahashi S et al (2011); Burke D T, et al (1987)).

Therefore in the context of the present invention a one or more polynucleotide which undergoes an overlap-extension PCR reaction is a polynucleotide which comprises a polynucleotide sequence to be extended in the overlap-extension PCR reaction.

In the context of the present invention the terms “overlap-extension PCR” (OE-PCR), “PCR-mediated overlap extension”, “PCR-based overlap extension”, “splicing by overlap extension (SOE)”, “gene splicing by overlap extension”, “splicing by overlap extension PCR” (SOE-PCR)”, “gene splicing by overlap extension PCR”, “gene splicing by PCR-mediated overlap extension”, “gene splicing by PCR-driven overlap extension”, and similar wording have the same meaning and are used interchangeably herewith. Overlap extension PCR (abbreviated herewith as OE-PCR) is a polymerase chain reaction-based technique well known to those skilled in the art and developed in the late 1980's (see Horton et al.). The technique is described in among others the above-mentioned Horton et al., and in Heckman and Pease. The mechanism of overlap extension PCR is for example described in FIG. 1 of Horton et al (herewith incorporated by reference) and in FIG. 1b of Heckman and Pease (herewith incorporated by reference).

In one embodiment, said performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-established order to yield a vector, preferably a circular vector comprising one or more functional guide-polynucleotide expression cassettes, comprises the steps of:

a) subjecting in each reaction one of said one or more polynucleotides and one suitable polynucleotide primer to overlap-extension PCR reaction, wherein the 3′-terminus of said polynucleotide primer contains a complementary sequence to the 3′-end of the polynucleotide sequence to be extended comprised in said one or more polynucleotide or wherein the 3′-terminus of the complement of said polynucleotide primer contains a complementary sequence to the 3′-end of the complement of the polynucleotide sequence to be extended comprised in said one or more polynucleotide, respectively, to yield one hybrid linear polynucleotide having a sequence comprising the polynucleotide sequence to be extended spliced at its 3′-terminus or at its 5′-terminus, respectively, with the polynucleotide sequence of the suitable primer,

b) optionally subjecting the hybrid linear polynucleotide obtained in step a) and another suitable primer to a second overlap-extension PCR reaction, wherein the 3′-terminus of said polynucleotide primer contains a complementary sequence to the 3′-end of the hybrid linear polynucleotide having a sequence comprising the polynucleotide sequence to be extended spliced at its 3′-terminus or at its 5′-terminus, respectively, with the polynucleotide sequence of the first suitable primer, or wherein the 3′-terminus of the complement of the other polynucleotide primer contains a complementary sequence to the 3′-end of the complement of the hybrid linear polynucleotide having a sequence comprising the polynucleotide sequence to be extended spliced at its 3′-terminus or at its 5′-terminus, respectively, with the polynucleotide sequence of the first suitable primer, respectively, to yield one hybrid linear polynucleotide having a sequence comprising the polynucleotide sequence to be extended spliced at its 3′-terminus and at its 5′-terminus, respectively, with the polynucleotide sequences of the suitable primers,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

The one or more polynucleotide sequence to be extended and the suitable polynucleotide primers used in the overlap extension PCR reaction are selected so that

1) each hybrid linear polynucleotide obtained in the one or more overlap-extension PCR reactions comprises at least a polynucleotide coding for a guide sequence;

2) said one or more hybrid linear polynucleotides obtained in the one or more overlap-extension PCR reaction comprise suitable 5′-termini and 3′-termini which allow assembly of the one or more hybrid linear polynucleotides and optionally of one or more additional linear polynucleotides with each other to yield a vector, preferably a circular vector comprising one or more functional guide-polynucleotide expression cassettes

3) said one or more hybrid linear polynucleotides obtained in the one or more overlap-extension PCR reaction comprise suitable 5′-termini and 3′-termini which allow assembly in vivo of the one or more hybrid linear polynucleotides and optionally of one or more additional linear polynucleotides with each other in a pre-defined order.

In order to satisfy condition 1), the polynucleotide sequence to be extended, comprising at the 5′-end and/or at the 3′-end to be extended a fragment of a guide-polynucleotide sequence may comprise a polynucleotide coding for a guide polynucleotide sequence. Alternatively in order to satisfy condition 1) a suitable primer used in the overlap extension PCR reaction may comprise a polynucleotide coding for a guide sequence. In yet another embodiment both the polynucleotide sequence to be extended and the suitable primer used in the overlap extension PCR reaction comprise a part of a polynucleotide coding for a guide sequence capable to yield the full polynucleotide coding for the guide sequence upon splicing of the two sequences in the OE-PCR reaction. In one preferred embodiment the fragment of a guide-polynucleotide expression cassette at the 5′-end and/or at the 3′-end of the polynucleotide sequence to be extended, does not comprise a polynucleotide coding for a guide sequence. In one preferred embodiment a suitable primer used in the overlap extension PCR reaction comprises a polynucleotide coding for a guide sequence.

In order to satisfy condition 2), the polynucleotide sequence to be extended in the one or more OE-PCR reaction, comprises at the 5′-end and/or at the 3′-end to be extended a fragment of a guide-polynucleotide expression cassette and the primer or primers used in the one or more OE-PCR reaction may comprise a second fragment of said guide-polynucleotide expression cassette such that upon OE-PCR the hybrid linear polynucleotide obtained comprises at least one functional guide-polynucleotide expression cassette at its 5′-terminus or at its 3′-terminus. Alternatively, the polynucleotide sequence to be extended in the one or more OE-PCR reaction, comprises at the 5′-end and at the 3′-end to be extended a fragment of a guide-polynucleotide expression cassette and the primers used in the one or more OE-PCR reaction may comprise a second fragment of said guide-polynucleotide expression cassette such that upon OE-PCR the hybrid linear polynucleotide obtained comprises a functional guide-polynucleotide both at the 5′-terminus and at the 3′-terminus. In yet an alternative embodiment the hybrid linear polynucleotides obtained in the one or more overlap-extension PCR reactions comprise at its 5′-terminus and/or 3′-terminus a fragment of a guide polynucleotide expression cassette and functional expression cassette are generated upon assembly of the one or more hybrid linear polynucleotides with each other and optionally with one or more additional linear polynucleotide. Therefore in one embodiment of the invention the hybrid linear polynucleotide obtained in the overlap-extension PCR reaction does not comprise a functional guide-polynucleotide expression cassette. The hybrid linear polynucleotide preferably comprises a fragment of a guide-polynucleotide expression cassette which fragment comprises a polynucleotide coding for a guide sequence.

In the context of the present invention said one or more additional linear polynucleotide is a linear polynucleotide defined as herein before which do not comprise a nucleotide coding for a guide-sequence.

In order to satisfy condition 3) the 5′-termini and 3′-termini present in the one or more hybrid linear polynucleotides and optionally in the one or more additional linear polynucleotides to be assembled are designed so as to allow homologous recombination in vivo between the one or more hybrid linear polynucleotides and optionally the one or more additional linear polynucleotides in a pre-defined order, leading to the formation of one type of product via homologous recombination.

Suitable primers are used in the OE-PCR reaction together with the one or more polynucleotides to yield hybrid linear polynucleotides as defined herewith above.

In one embodiment of a method to produce a vector, preferably a circular vector according to the invention primers used in each overlap-extension PCR reaction are at most 100 nucleotides long, preferably at most 80, 70, 60, 50, 40, 30 nucleotides long. In another embodiment of a method to produce a circular vector according to the invention primers used in each overlap-extension PCR reaction are at least 30 nucleotides long, preferably at least 40, 50, 60 nucleotides long.

The primers used in the method to produce a vector, preferably a circular vector according to the invention are selected, such as selected and designed, so that so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

Therefore in one embodiment the suitable primers used in the OE-PCR reaction comprises at least a polynucleotide coding for a guide sequence. In another embodiment the suitable primers used in the OE-PCR reaction comprises at least a polynucleotide coding for a guide sequence and a terminus, such as a 3′-terminus containing a complementary sequence to the 3′-end of the polynucleotide sequence to be extended. In another embodiment the suitable primers used in the OE-PCR reaction comprises at least a polynucleotide coding for a guide sequence and a terminus, such as a 3′-terminus containing a complementary sequence to the 3′-end of a hybrid polynucleotide. In yet another embodiment the suitable primers used in the OE-PCR reaction comprises comprises at least a polynucleotide coding for a guide sequence, a 3′-terminus containing a complementary sequence to the 3′-end of the polynucleotide sequence to be extended or a 3′-terminus containing a complementary sequence to the 3′-end of a hybrid polynucleotide to be extended, and a 5′-terminus sharing homologous sequences with the 3′-terminus of a hybrid linear polynucleotide or additional linear polynucleotide to be used in the assembly step.

The last step of the method to produce a vector, preferably a circular vector according to the first aspect comprises:
subjecting the one or more hybrid linear polynucleotides obtained in the overlap-extension PCR and optionally one or more additional linear polynucleotides to an assembly reaction yielding a vector, preferably a circular vector comprising one or more functional guide-polynucleotide expression cassettes,

wherein the assembly reaction occurs in vivo.

According to the invention the assembly reaction occurs in a host cell. In other words according to the invention the assembly reaction occurs in vivo. Assembly of the one or more hybrid linear polynucleotides obtained in the overlap-extension PCR and optionally one or more additional linear polynucleotides will occur in vivo by homologous recombination. This step exploits the homologous recombination-mediated double strand brake DNA repair pathway in the host cell where the assembly reaction occurs. A suitable host cell where the assembly reaction may occur is S. cerevisiae. Methods to produce linear or circular vectors exploiting homologous recombination mediated DNA repair pathways are know to those skilled in the art and e.g. described in Raymond et al.

“Homologous recombination” refers to a reaction between nucleotide sequences having corresponding sites containing a similar nucleotide sequence (i.e., homologous sequences) through which the molecules can interact (recombine) to form a new, recombinant nucleic acid sequence. The sites of similar nucleotide sequence are each referred to herein as a “homologous sequence”. Generally, the frequency of homologous recombination increases as the length of the homology sequence increases. Thus, while homologous recombination can occur between two nucleic acid sequences that are less than identical, the recombination frequency (or efficiency) declines as the divergence between the two sequences increases.

Recombination may be accomplished using one homology sequence on each of two molecules to be combined, thereby generating a “single-crossover” recombination product. Alternatively, two homology sequences may be placed on each of two molecules to be recombined. Recombination between two homology sequences on the donor with two homology sequences on the target generates a “double-crossover” recombination product.

Therefore in order for two polynucleotide sequences to be recombined by homologous recombination with each other, both polynucleotides need to share a region of homology with each other. These regions of homology are called interchangeably herewith “flanking regions”, “flanking sequences”, “overlapping regions”, “overlapping sequences”, “homologous regions”, “homologous sequences”. In order for homologous recombination to take place the homologous sequences do not need to be identical. However the efficiency of homologous recombination increases with the level of sequence identity between the homolgous sequences. Preferably the homologous sequence will be at least 50% identical, preferably at least 60%, 70%, 80%, 85%, 90%, 95%, identical with each other, more preferably the homologous sequences will be 100% identical with each other. It is known to those skilled in the art that efficiency of homologous recombination increases with the length of the homologous sequences between the polynucleotides to be recombined. In one embodiment the homologous sequences are at least 10 bp long, preferably at least 20 bp, 30 bp, 40 bp, 50 bp, 100 bp, 500 bp, 1000 bp or more. It is known to those skilled in the art that while in S. cerevisiae the efficiency of homologous recombination is very high as double strand DNA brakes are mainly repaired by this mechanism, in other host cell homologous recombination is less efficient, requiring longer homologous sequences. In filamentous fungi said homologous sequences may be several thousands of nucleobases long. The skilled person will know how to design sufficiently long homologous sequences sharing sufficient sequence identity to allow efficient homologous recombination. Preferably the homologous sequences will share at least 80% sequence identity and will be at least 20 bp long. In a preferred embodiment the assembly reaction in vivo is performed in a host cell belonging to S. cerevisiae.

In an embodiment of the method to produce a vector, preferably a circular vector comprising one or more guide-polynucleotide expression cassettes according to the first aspect, the one or more polynucleotide sequence to be extended or additional linear polynucleotide comprises one or more elements selected from the group consisting of: an origin of replication or a fragment thereof, a selectable marker or a fragment thereof, a CAS9 expression cassette or a fragment thereof, a donor polynucleotide or a fragment thereof. In another embodiment of the method to produce a vector, preferably a circular vector comprising one or more guide-polynucleotide expression cassettes according to the first aspect, the one or more polynucleotide sequence to be extended or additional linear polynucleotide comprises one or more elements selected from the group consisting of: an origin of replication or a fragment thereof, a selectable marker or a fragment thereof, a CAS9 expression cassette or a fragment thereof, a donor polynucleotide or a fragment thereof, a control sequence or fragment thereof. Preferably the one or more elements comprise standardized and/or re-usable vector elements namely, standardized origin of replications or fragments thereof, standardised selectable marker cassettes or fragments thereof, standardised control sequences (e.g. standardised promoters or terminators) or fragments thereof.

An “origin of replication” is herewith defined as a sequence of DNA at which replication is initiated on a chromosome, plasmid or virus. The term “origin of replication” or “plasmid replicator” or “replicon” is defined herein as a nucleotide sequence that enables a plasmid or vector to replicate in vivo. For the vector according to the invention to be maintained in a host cell an origin of replication (ORI), also know as autonomously replicating sequence (ARS) or replicon is necessary. The autonomous replication sequence may be any suitable sequence available to the person skilled in the art that allows for plasmid replication that is independent of chromosomal replication. The origin of replication may be any plasmid replicator mediating autonomous replication that functions in a cell. Examples of bacterial origins of replication are the origins of replication of plasmids pBR322, pUC19, pACYC177, and pACYC184 permitting replication in E. coli, RSF1010 permitting replication in Pseudomonas is described, e.g., by F. Heffron et al., in Proc. Nat'l Acad. Sci. USA 72(9):3623-27 (September 1975), and pUB110, pE194, pTA1060, and pAM131 permitting replication in Bacillus. For the efficient expression of gRNA in yeast an ORI which leads to high copy number of the resulting plasmid is highly desirable. Preferred origin of replications used in S. cerevisiae is the 2-micron (2μ) plasmid sequence (Broach et al.). The autonomous replication sequence may be any suitable sequence available to the person skilled in the art that it confers to the plasmid replication that is independent of chromosomal replication. Preferably, the autonomous replication sequence is the AMA1 replicon (Gems et al.).

A “selectable marker” is a gene which permit easy selection of transformed cells, the product of which provides for biocide or viral resistance, resistance to heavy metals, prototrophy to auxotrophs, and the like. The selectable marker may be introduced into the cell on the vector as an expression cassette or may be introduced on a separate vector. Some chromogenic or fluorescent protein may be used as selectable markers. There are many varieties of chromogenic or fluorescent proteins such as Red Fluorescent Protein (RFP), Green Fluorescent Protein (GFP), mCherry, dsRed, etcetera.

A selectable marker for use in a filamentous fungal cell may be selected from the group including, but not limited to, amdS (acetamidase), argB (ornithine carbamoyltransferase), bar (phosphinothricinacetyltransferase), bleA (phleomycin binding), hygB (hygromycinphosphotransferase), niaD (nitrate reductase), pyrG (orotidine-5′-phosphate decarboxylase), sC (sulfate adenyltransferase), NAT or NTC (Nourseothricin) and trpC (anthranilate synthase), KanMX (resistance to G418/geneticin; the selection marker kanMX is a hybrid gene consisting of a bacterial aminoglycoside phosphotransferase (kanr from transposon Tn903) under control of the strong TEF promoter from Ashbya gossypii; mammalian cells, yeast, and other eukaryotes acquire resistance to geneticin (=G418, an aminoglycoside antibiotic similar to kanamycin) when transformed with a kanMX marker; in yeast, the kanMX marker avoids the requirement of auxotrophic markers; in addition, the kanMX marker renders E. coli resistant to kanamycin.) as well as equivalents from other species. Preferred for use in an Aspergillus and Penicillium cell are the amdS (see for example EP 635574 B1, EP0758020A2, EP1799821A2, WO 97/06261A2) and pyrG genes of A. nidulans or A. oryzae and the bar gene of Streptomyces hygroscopicus. More preferably an amdS gene is used, even more preferably an amdS gene from A. nidulans or A. niger. A most preferred selectable marker gene is the A. nidulans amdS coding sequence fused to the A. nidulans gpdA promoter (see EP 635574 B1). Other preferred AmdS markers are those described in WO2006/040358. AmdS genes from other filamentous fungi may also be used (WO 97/06261).

Markers which can be used in a prokaryotic host cell include ATP synthetase, subunit 9 (oliC), orotidine-5′-phosphatedecarboxylase (pvrA), the ampicillin resistance gene (E. coli), resistance genes for neomycin, kanamycin, tetracycline, spectinomycin, erythromycin, chloramphenicol, phleomycin (Bacillus) and the E. coli uidA gene, coding for β-glucuronidase (GUS). Vectors may be used in vitro, for example for the in vitro production of RNA in an in vitro transcription system or used to transfect or transform a host cell.

Versatile marker genes that can be used for transformation of most filamentous fungi and yeasts such as acetamidase genes or cDNAs (the amdS, niaD, facA genes or cDNAs from A. nidulans, A. oryzae or A. niger), or genes providing resistance to antibiotics like G418, hygromycin, bleomycin, kanamycin, methotrexate, phleomycin orbenomyl resistance (benA). Alternatively, specific selection markers can be used such as auxotrophic markers which require corresponding mutant host strains: e. g. D-alanine racemase (from Bacillus), URA3 (from S. cerevisiae or analogous genes from other yeasts), pyrG or pyrA (from A. nidulans or A. niger), argB (from A. nidulans or A. niger) or trpC.

The procedures used to ligate elements described above to construct a vector according to the present invention are well known to one skilled in the art (see, e.g. Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995).

A “CAS9 expression cassette” is a polynucleotide sequence comprising a polynucleotide coding for a CAS9 protein operably linked to control sequences which allow expression of said protein in a suitable host cell. Control sequences and operably linked have been herewith above defined.

A Cas protein in the context of all embodiments of the present invention refers to any Cas protein suitable for the purpose of the invention. A Cas protein may comprise enzymatic activity or may not comprise enzymatic activity. Non-limiting examples of Cas proteins include CasI, CasI B, Cas2, Cas3, Cas4, Cas5, Cas6, Cas7, Cas8, Cas9 (also known as CsnI and CsxI2), CasIO, CsyI, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CsbI, Csb2, Csb3, CsxI7, CsxI4, CsxIO, CsxI6, CsaX, Csx3, CsxI, CsxIS, CsfI, Csf2, Csf3, Csf4, homologs thereof or modified versions thereof (Slaymaker et al 2016; Kleinstiver et al 2016; Nelson and Gersbach 2016). In alternative to a Cas protein as described herein above, a Cpf1 protein (as described in Zetsche et al 2015) can also be used in the method according to the invention, optionally in combination with adaptation of the guide-polynucleotide expression cassette. These Cas proteins are known to the person skilled in the art; for example, the amino acid sequence of S. pyogenes Cas9 protein may be found in the SwissProt database under accession number Q99ZW2. Preferably, an unmodified Cas protein according to the present invention has DNA cleavage activity, such as e.g. Cas9. Preferably, a Cas protein according is Cas9, and may be Cas9 from S. pyogenes or S. pneumoniae or a Cas9 from Staphylococcus aureus (Ran et al 2015). Preferably, a Cas protein according to the present invention directs cleavage of one or both polynucleotide strands at the location of the target-polynucleotide, such as within the target-polynucleotide and/or within the reverse complement of the target-polynucleotide. At the location of the target-polynucleotide is herein defined as within about 1, 2, 3, 4, 5, 6, 7, 8, 9, 10, 15, 20, 25, 50, 100, 200, 500, or more nucleotides from the first or last nucleotide of a target-polynucleotide; more preferably, within 1,2, 3, 4, 5, 6, 7, 8, 9, 10, 15, 20, 25, 50, 100, 200, 500, or more nucleotides from the first or last nucleotide of a target-polynucleotide; even more preferably, within 1, 2, 3, 4, 5, 6, 7, 8, 9, 10, 15, 20, 25, 50 nucleotides from the first or last nucleotide of a target-polynucleotide. Accordingly, a Cas protein according to the present invention preferably directs cleavage of one or both polynucleotide strands within about 1, 2, 3, 4, 5, 6, 7, 8, 9, 10, 15, 20, 25, 50, 100, 200, 500, or more nucleotides from the first or last nucleotide of a target-polynucleotide; more preferably, within 1,2, 3, 4, 5, 6, 7, 8, 9, 10, 15, 20, 25, 50, 100, 200, 500, or more nucleotides from the first or last nucleotide of a target-polynucleotide; even more preferably, within 1, 2, 3, 4, 5, 6, 7, 8, 9, 10, 15, 20, 25, 50 nucleotides from the first or last nucleotide of a target-polynucleotide. Typically, a target-polynucleotide according to the present invention is associated with a PAM sequence (defined elsewhere herein) and the PAM sequence is preferably immediately downstream (3′) of the target-sequence; the formation of the CRISPR-Cas complex typically results in cleavage of one or both polynucleotide strands 3 base pairs upstream (5′) of the PAM sequence.

Preferably, a Cas protein has activity for directing cleavage of both polynucleotide strands at the location of the target-polynucleotide. Cas nuclease activity is typically performed by two separate catalytic domains, namely RuvC and HNH. Each domain cuts one polynucleotide strand each domain can be inactivated by a single point mutation. A Cas protein as defined herewith may thus conveniently be mutated with respect to a corresponding wild-type Cas protein such that the mutated Cas protein has altered nuclease activity and lacks the ability to cleave one or both strands of a target-polynucleotide. For example, an aspartate-to-alanine substitution (D10A) in the RuvC I catalytic domain of Cas9 from S. pyogenes converts Cas9 from a nuclease that cleaves both strands to a nickase, which is herein defined as a Cas protein that cleaves a single strand of a target-polynucleotide. Other examples of mutations that render Cas9 into a nickase include, but are not limited to H840A, N854A, and N863A. In the context of the present invention, a Cas protein having nickase activity may be used for genome editing via homologous recombination, preferably the double nicking technique according to Ran et al., 2013. Accordingly, a preferred Cas protein according to the present invention comprises at least one mutation, such that the protein has altered nuclease activity compared to the corresponding wild-type Cas protein, preferably having activity to direct cleavage of a single polynucleotide strand at the location of the target-sequence. Such so-called nickase mutant can conveniently be used in duplex set-up, i.e. in a composition according to the present invention comprising a Cas protein nickase mutant with RuvC mutated and a Cas protein nickase mutant wherein NHN is mutated, such that the one Cas protein mutant nicks one strand of the polynucleotide target and the other Cas protein mutant nicks the other strand of the polynucleotide target. Depending on the two guide-polynucleotides used, the two different CRISPR-Cas complexes will effectively result in two single-strand nicks in the polynucleotide target; these nicks may be several nucleotides up to 5, 10, 20, 30 or more apart. Such double nicking method greatly enhances specificity of NEJH. Background information on double nicking can be found in e.g. Ran et al, 2013.

A Cas protein according to the present invention may comprise two or more mutated catalytic domains of Cas9, such as RuvC I, RuvC II and/or RuvC III to result in a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. Preferably, a Cas protein is considered to substantially lack all DNA cleavage activity when the DNA cleavage activity of the mutated enzyme is less than about 25%, 10%, 5%, 1%, 0.1%, 0.01%, or lower with respect to its non-mutated form. A Cas protein lacking substantially all enzyme activity can conveniently be used for gene silencing or down regulation of expression since the CRISPR-CAS complex will hamper transcription from the target-polynucleotide. Other mutations may be useful; where the Cas9 or other Cas protein is from a species other than S. pyogenes, mutations in corresponding amino acids may be made to achieve similar effects; the person skilled in the art knows how to identify these corresponding amino acids.

A Cas protein according to the present invention may be a fusion protein and comprise at least one heterologous functional domain, such domain preferably is a domain comprising FokI activity such as described by Aggarwal et al (Aggarwal, A. K.; Wah, D. A.; Hirsch, J. A.; Dorner, L. F.; Schildkraut, I. (1997). “Structure of the multimodular endonuclease FokI bound to DNA”. Nature 388 (6637): 97-100). The enzyme FokI is naturally found in Flavobacterium okeanokoites and is a bacterial type IIS restriction endonuclease consisting of an N-terminal DNA-binding domain and a non-specific DNA cleavage domain at the C-terminal (Durai et al., 2005). When the FokI protein is bound to double stranded DNA via its DNA-binding domain at the 5′-GGATG-3′:3′-CATCC-5′ recognition site, the DNA cleavage domain is activated and cleaves, without further sequence specificity, the first strand 9 nucleotides downstream and the second strand 13 nucleotides upstream of the nearest nucleotide of the recognition site (Wah et al., 1998. Cas9-FokI fusions have been described inter alia in Guilinger et al., 2014; and in Tsai et al., 2014.

A Cas fusion protein according to the present invention may comprise 1, 2, 3, 4, 5, 6, 7, 8, 9, 10, or more domains in addition to the Cas protein. Examples of protein domains that may be fused to a Cas protein include, but are not limited to, epitope tags, reporter gene sequences, and protein domains having one or more of the following activities: methylase activity, demethylase activity, transcription activation activity, transcription repression activity, transcription release factor activity, historic modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A Cas protein may be fused to a gene sequence encoding a protein or a fragment of a protein that bind DNA molecules or bind other cellular molecules, including but not limited to, maltose binding protein (MBP), S-tag, Lex A DNA binding domain (DBD) fusions, GAL4 DNA binding domain fusions, and herpes simplex virus (HSV) BP 16 protein fusions. Additional domains that may form part of a fusion protein comprising a CRISPR enzyme are described in US20110059502. A tagged Cas protein may be used to identify the location of a target-polynucleotide. A preferred Cas fusion protein according to the present invention comprises a FokI domain as defined here above.

A preferred Cas protein according to the present invention comprises a nuclear localization sequence, preferably a heterologous nuclear localization sequence. Such nuclear localization sequence is also referred as a nuclear localization signal. Preferably, such nuclear localization signal confers to the CRISPR-Cas complex sufficient strength to drive accumulation of said CRISPR-Cas complex in a detectable amount in the nucleus of a host cell. Without wishing to be bound by theory, it is believed that a nuclear localization sequence is not necessary for CRISPR-Cas activity in a host cell, but that including such sequences enhances activity of the system, especially as to targeting nucleic acid molecules into the nucleus. Such nuclear localization sequence is preferably present in the Cas protein, but may also be present anywhere else such that targeting of the CRISPR-Cas system to the nucleus is facilitated. A preferred nuclear localization sequence is the SV40 nuclear localization sequence. A polynucleotide coding for a Cas protein, e.g. a polynucleotide coding for a Cas9 protein, is preferably codon optimized for the host cell it is to be expressed in, more preferably the Cas protein encoding polynucleotide is codon pair optimized. In general, codon optimization refers to a process of modifying a nucleic acid sequence for enhanced expression in a host cell of interest by replacing at least one codon (e.g. more than 1, 2, 3, 4, 5, 10, 15, 20, 25, 50, or more codons) of a native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See e.g. Nakamura, Y., et al., 2000. Computer algorithms for codon optimizing a particular sequence for expression in a particular host cell are also available, such as Gene Forge (Aptagen; Jacobus, Pa.), are also available. Preferably, one or more codons (e.g. 1, 2, 3, 4, 5, 10, 15, 20, 25, 50, or more, or all codons) in a sequence encoding a Cas protein correspond to the most frequently used codon for a particular amino acid. Preferred methods for codon optimization are described in WO2006/077258 and WO2008/000632). WO2008/000632 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence.

A “donor polynucleotide” as defined herewith is an exogenous polynucleotide, also referred to as donor DNA when the polynucleotide is a DNA, or repair template, that upon cleavage of the target-polynucleotide by the CRISPR-Cas complex recombines with the target-polynucleotide, resulting in a modified target-polynucleotide. Such exogenous polynucleotide is herein referred to as an exogenous polynucleotide or donor nucleotide according to the present invention and may be single-stranded or double-stranded. The exogenous polynucleotide may be present on a vector or may be present as such, may be encoded by another polynucleotide or may be operably linked to the guide-polynucleotide and may have sequence identity to a part of the target-polynucleotide upstream of the PAM associated with the guide-sequence (i.e. on the 5′ side of the PAM) or may have sequence identity to a part of the target-polynucleotide downstream of the PAM associated with the guide-sequence (i.e. on the 5′ side of the PAM). The donor polynucleotide may be comprised in a separate vector. A vector carrying an exogenous polynucleotide may be any vector described herein below. The exogenous polynucleotide may be present on a vector that comprises a polynucleotide encoding a Cas protein as defined herewith and/or comprising a guide-polynucleotide or a polynucleotide encoding a guide-polynucleotide as defined herewith.

In one embodiment according to the method to produce a vector, preferably a circular vector comprising one or more guide polynucleotide expression cassettes, the vector or circular vector obtained after assembly comprises one or more elements selected from the group consisting of: an origin of replication, a selectable marker, a CAS9 expression cassette, a donor polynucleotide or a combination of one or more of said elements.

In yet another embodiment according to the method to produce a vector, preferably a circular vector comprising one or more guide polynucleotide expression cassettes, the vector or circular vector obtained after assembly comprises an origin of replication, preferably an origin of replication and a selectable marker, even more preferably an origin of replication, a selectable marker and a CAS 9 expression cassette and/or a donor polynucleotide.

Accordingly, in an embodiment, the present invention provides for a vector according to the present invention wherein a polynucleotide encoding a Cas protein, a guide-polynucleotide or a polynucleotide encoding a guide-polynucleotide are present on a single vector, which may further comprise any elements necessary for expressing the encoded products such as promoter and terminator elements. Such single (all-in-one) vector has the advantage that all components necessary for a CRISPR-Cas system are present together; in addition, a single transformation event, optionally in combination with a donor polynucleotide, suffices to introduce the components into a host cell.

In one embodiment of the method to produce a vector, preferably a circular vector according to the first aspect the vector, preferably the circular vector comprises one guide-polynucleotide expression cassette

Therefore in one embodiment of the first aspect the invention provides a method to produce a vector, preferably a circular vector comprising one guide-polynucleotide expression cassettes, wherein said one guide-polynucleotide expression cassettes comprise a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method preferably comprises the following steps:
providing one polynucleotide, wherein said polynucleotide comprises a polynucleotide sequence to be extended, wherein the polynucleotide sequence to be extended comprises at the 5′-end and/or at the 3′-end to be extended a fragment of a guide-polynucleotide expression cassette;
performing one overlap-extension PCR reactions by subjecting in the reaction said polynucleotide and two suitable polynucleotide primers to yield one hybrid linear polynucleotide,

wherein the polynucleotide sequence to be extended and the suitable polynucleotide primers are selected so that the hybrid linear polynucleotide obtained in the overlap-extension PCR reaction comprises at least a polynucleotide coding for a guide sequence and suitable 5′-termini and 3′-termini which allow assembly in vivo of the hybrid linear polynucleotides and optionally of one or more additional linear polynucleotides with each other in a pre-defined order to yield a vector, preferably a circular vector comprising one or more functional guide-polynucleotide expression cassettes;
subjecting the hybrid linear polynucleotide obtained in the overlap-extension PCR and optionally one or more additional linear polynucleotides to an assembly reaction yielding a vector, preferably a circular vector comprising one or more functional guide-polynucleotide expression cassettes,

preferably wherein the assembly reaction occurs in vivo.

In another embodiment of the method to produce a vector, preferably a circular vector according to the first aspect the vector, preferably the circular vector comprises two or more guide-polynucleotide expression cassettes.

Therefore in one embodiment of the first aspect the invention provides a method to produce a vector, preferably a circular vector comprising two or more guide-polynucleotide expression cassettes, wherein said two or more guide-polynucleotide expression cassettes comprise a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method preferably comprises the following steps:
providing two or more polynucleotides, wherein the two or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing two or more overlap-extension PCR reactions by subjecting in each reaction one of said two or more polynucleotides and two suitable polynucleotide primers to yield one hybrid linear polynucleotide,

wherein the two or more polynucleotide sequence to be extended and the suitable polynucleotide primers are selected so that each hybrid linear polynucleotide obtained in the two or more overlap-extension PCR reactions comprises at least a polynucleotide coding for a guide sequence and suitable 5′-termini and 3′-termini which allow assembly in vivo of the two or more hybrid linear polynucleotides and optionally of one or more additional linear polynucleotides with each other in a pre-defined order to yield a vector, preferably a circular vector comprising one or more functional guide-polynucleotide expression cassettes;
subjecting the two or more hybrid linear polynucleotides obtained in the overlap-extension PCR and optionally one or more additional linear polynucleotides to an assembly reaction yielding a vector, preferably a circular vector comprising two or more functional guide-polynucleotide expression cassettes,

preferably wherein the assembly reaction occurs in vivo.

In a second aspect, the present invention provides a method of assembling a vector, preferably a circular vector comprising one or more guide-polynucleotide expression cassettes in vivo, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
providing one or more linear polynucleotides, wherein said one or more linear polynucleotides comprise at the 5′-terminus and/or at the 3′-terminus at least one guide-polynucleotide expression cassette or a fragment thereof comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes;
optionally selecting a host cell comprising a vector, preferably a circular vector and wherein said vector comprises one or more functional guide-polynucleotide expression cassettes;
optionally recovering the vector, preferably the circular vector.

In a first step a method of assembling a vector, preferably a circular vector comprising one or more guide-polynucleotide expression cassettes in vivo according to the second aspect of the invention comprises:
providing one or more linear polynucleotides, wherein said one or more linear polynucleotides comprise at the 5′-terminus and/or at the 3′-terminus at least one guide-polynucleotide expression cassette or a fragment thereof comprising at least the guide sequence.

A guide-polynucleotide expression cassette, a CRISPR-CAS complex, a guide-polynucleotide, a guide-sequence, a target-polynucleotide, a CRISPR-CAS system, polynucleotide, a control sequence, “operably linked”, promoter, fragment of a guide-polynucleotide expression cassette, homologous recombination, homologous sequence, have been defined herewith above in the first aspect of the invention. These definitions are valid throughout the whole specification.

The one or more linear polynucleotides are polynucleotides as defined herewith which comprise at the 5′-terminus and/or at the 3′-terminus at least one guide-polynucleotide expression cassette or a fragment thereof comprising at least the guide sequence.

The one or more linear polynucleotides, the one or more polynucleotides comprising a polynucleotide sequence to be extended and the one ore more additional linear polynucleotides as defined herewith may be produced according to methods known in the art. E.g the polynucleotides suitable for use in the invention may typically be generated by any amplification process known in the art (e.g., PCR, RT-PCR and the like).

The one or more linear polynucleotides and optionally the one or more additional polynucleotides used in the second aspect may comprise one or more elements selected from the group consisting of: an origin of replication or a fragment thereof, a selectable marker or a fragment thereof, a CAS9 expression cassette or a fragment thereof, a donor polynucleotide or a fragment thereof, as defined herein before.

In a second step, a method of assembling a vector, preferably a circular vector comprising one or more guide-polynucleotide expression cassettes in vivo according to the second aspect of the invention comprises:
transforming said one or more linear polynucleotides, and optionally one or more additional linear polynucleotides into a host cell.

Transformation of said one or more linear polynucleotides and optionally one or more additional linear polynucleotides into a host cell may occur according to various techniques known to those skilled in the art. Non-limiting examples of methods used to introduce heterologus nucleic acids into various organisms include; transformation, transfection, transduction, electroporation, ultrasound-mediated transformation, particle bombardment, microprojectile bombardment, protoplast method, Agrobacterium mediated transformation (AMT) and the like. In some instances the addition of carrier molecules can increase the uptake of DNA in cells typically thought to be difficult to transform by conventional methods. Conventional methods of transformation are readily available to the skilled person. Preferably the protoplast method is used for filamentous fungi. Procedures for transformation are inter alia described by J. R. S. Fincham, Transformation in fungi. 1989, Microbiological reviews. 53, 148-170. Transformation may involve a process consisting of protoplast formation, transformation of the protoplasts, and regeneration of the cell wall in a manner known per se. Suitable procedures for transformation of Aspergillus cells are described in EP 238 023 and Yelton et al., 1984, Proceedings of the National Academy of Sciences USA 81:1470-1474. Suitable procedures for transformation of Aspergillus and other filamentous fungal host cells using Agrobacterium tumefaciens are described in e.g. De Groot et al., Agrobacterium tumefaciens-mediated transformation of filamentous fungi. Nat Biotechnol. 1998, 16:839-842. Erratum in: Nat Biotechnol 1998 16:1074. A suitable method of transforming Fusarium species is described by Malardier et al., 1989, Gene 78:147156 or in WO 96/00787. Other methods can be applied such as a method using biolistic transformation as described in: Christiansen et al., Biolistic transformation of the obligate plant pathogenic fungus, Erysiphe graminis f. sp. hordei. 1995, Curr Genet. 29:100-102. Yeast may be transformed using any method known in the art such as the procedures described by Becker and Guarente, In Abelson, J. N. and Simon, 1983; Hinnen et al., 1978, and Gietz R D, Woods R A. 2002.

In a third step, a method of assembling a vector, preferably a circular vector comprising one or more guide-polynucleotide expression cassettes in vivo according to the second aspect of the invention comprises:
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes.

The one more more linear polynucleotides and one more more additional linear polynucleotides have been selected to comprise suitable 5′-termini and 3′-termini which allow homologous recombination of said one or more linear polynucleotides and optionally of said one or more additional linear polynucleotides with each other in a pre-established order. How to select linear polynucleotides and additional linear polynucleotides to comprise suitable 5′-termini and 3′-termini to allow homologous recombination is performed by providing the two polynucleotide sequences to be recombined by homologous recombination with each other, with homologous sequences as defined herein before. Preferably the homologous sequence will be at least 50% identical, preferably at least 60%, 70%, 80%, 85%, 90%, 95%, identical with each other, more preferably the homologous sequences will be 100% identical with each other. In one embodiment the homologous sequences are at least 10 bp long, preferably at least 20 bp, 30 bp, 40 bp, 50 bp, 100 bp, 500 bp, 1000 bp or more. The skilled person will know how to design sufficiently long homologous sequences sharing sufficient sequence identity to allow efficient homologous recombination in the host cell. Preferably the homologous sequences will share at least 80% sequence identity and will be at least 20 bp long. In a preferred embodiment the method of the second aspect of the invention is performed in a host cell belonging to S. cerevisiae.

In a fourth optional step, a method of assembling a vector, preferably a circular vector comprising one or more guide-polynucleotide expression cassettes in vivo according to the second aspect of the invention comprises:
optionally selecting a host cell comprising a vector, preferably a circular vector and wherein said vector comprises one or more functional guide-polynucleotide expression cassettes.

In a fifth optional step, a method of assembling a vector, preferably a circular vector comprising one or more guide-polynucleotide expression cassettes in vivo according to the second aspect of the invention comprises:
optionally recovering the vector, preferably the circular vector.

Selection of host cells comprising the vector, preferably the circular vector may be performed with various techniques know to those skilled in the art. Typically the one or more linear polynucleotides and optionally one or more additional linear polynucleotides used in the assembly reaction will comprise one or more selectable markers which are incorporated in the polynucleotide sequence of the circular vector and allow differential selection of host cells comprising a vector comprising all of the one or more functional guide-polynucleotide expression cassettes from other vectors.

Once the host cell comprising the vector, preferably the circular vector have been selected, the vector, preferably the circular vector may be recovered therefrom according to methods known to those skilled in the art. Typically host cell comprising the correct vector may be propagated in a suitable medium, after sufficient cell growth the culture may be harvested, host cell may be lysed and the vector, preferably the circular vector may be purified from other cellular components, e.g. by chromatography such as chromatography using silica gel or anion exchange resins. The vector can be further recovered after chromatography using standard techniques, such as ethanol precipitation.

In one embodiment of the method according to the second aspect the vector, preferably the circular vector comprises two or more guide-polynucleotide expression cassettes, preferably the method comprises the following steps:
providing two or more polynucleotides, wherein the two or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing two or more overlap-extension PCR reactions by subjecting in each reaction one of said two or more polynucleotides and two suitable polynucleotide primers to yield one hybrid linear polynucleotide,

wherein the two or more polynucleotide sequence to be extended and the suitable polynucleotide primers are selected so that each hybrid linear polynucleotide obtained in the two or more overlap-extension PCR reactions comprises at least a polynucleotide coding for a guide sequence and suitable 5′-termini and 3′-termini which allow assembly in vivo of the two or more hybrid linear polynucleotides and optionally of one or more additional linear polynucleotides with each other in a pre-defined order to yield a vector, preferably a circular vector comprising one or more functional guide-polynucleotide expression cassettes;
subjecting the two or more hybrid linear polynucleotides obtained in the overlap-extension PCR and optionally one or more additional linear polynucleotides to an assembly reaction yielding a vector, preferably a circular vector comprising two or more functional guide-polynucleotide expression cassettes,

preferably wherein the assembly reaction occurs in vivo.

In another embodiment in the method according to the second aspect the vector, preferably the circular vector comprises one guide-polynucleotide expression cassette, preferably wherein the method comprises the following steps:
providing one polynucleotide, wherein said polynucleotide comprises a polynucleotide sequence to be extended, wherein the polynucleotide sequence to be extended comprises at the 5′-end and/or at the 3′-end to be extended a fragment of a guide-polynucleotide expression cassette;
performing one overlap-extension PCR reactions by subjecting in the reaction said polynucleotide and two suitable polynucleotide primers to yield one hybrid linear polynucleotide,

wherein the polynucleotide sequence to be extended and the suitable polynucleotide primers are selected so that the hybrid linear polynucleotide obtained in the overlap-extension PCR reaction comprises at least a polynucleotide coding for a guide sequence and suitable 5′-termini and 3′-termini which allow assembly in vivo of the hybrid linear polynucleotides and optionally of one or more additional linear polynucleotides with each other in a pre-defined order to yield a vector, preferably a circular vector comprising one or more functional guide-polynucleotide expression cassettes;
subjecting the hybrid linear polynucleotide obtained in the overlap-extension PCR and optionally one or more additional linear polynucleotides to an assembly reaction yielding a vector, preferably a circular vector comprising one or more functional guide-polynucleotide expression cassettes,

preferably wherein the assembly reaction occurs in vivo.

In a third aspect the invention provides a method for producing a recombinant host cell comprising a vector, preferably a circular vector, wherein said vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
providing one or more linear polynucleotides, wherein said one or more linear polynucleotides comprise at the 5′-terminus and/or at the 3′-terminus at least one guide-polynucleotide expression cassette or a fragment thereof comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes;
optionally selecting a host cell comprising a circular vector and wherein said vector comprises one or more functional guide-polynucleotide expression cassettes.

The first four steps of the method according to the third aspect may be performed in analogy with the corresponding steps of the method according to the second aspect of the invention.

In one embodiment, the method according to the third aspect of the invention further comprises the steps of:
recovering the vector, preferably the circular vector;
transforming the vector, preferably the circular vector in a second host cell;
optionally isolating the second host cell comprising the vector, preferably the circular vector.

Recovery of the vector, preferably the circular vector in the method according to the third aspect, transformation of the vector, preferably the circular vector in a second host cell and isolation of the second host cell comprising the vector, preferably the circular vector may be performed according to methods analogous to those described herein above.

In a fourth aspect, the invention provides a method for producing a recombinant host cell comprising a vector, preferably a circular vector, wherein said vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and the guide-polynucleotide can direct binding of a Cas protein at a target-polynucleotide in a host cell to form a CRISPR-Cas complex, wherein the method comprises the following steps:
performing a method of assembling a circular vector comprising one or more guide-polynucleotide expression cassettes in vivo according to the second aspect in a first host cell, preferably a first host cell belonging to the species S. cerevisiae, wherein said first host cell comprising a vector, preferably a circular vector, is selected and the vector, preferably the circular vector is recovered, wherein said vector comprises one or more functional guide-polynucleotide expression cassettes,
transforming the vector, preferably the circular vector in a second host cell;
optionally isolating the second host cell comprising the vector, preferably the circular vector.

Recovery of the vector, preferably of the circular vector in the method according to the forth aspect, transformation of the vector in a second host cell and isolation of the second host cell comprising the vector may be performed according to methods analogous to those described herein above.

In one embodiment of the method according to the third or fourth aspect, the one or more linear polynucleotides are hybrid linear polynucleotides as defined herein before in the first aspect of the invention, obtained by
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

In a preferred embodiment of the method according to the third or forth aspect of the invention, said performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other to yield a vector, preferably a circular vector comprising one or more functional guide-polynucleotide expression cassettes, comprises the steps of:

a) subjecting in each reaction one of said one or more polynucleotides and one suitable polynucleotide primer to overlap-extension PCR reaction, wherein the 3′-terminus of said polynucleotide primer contains a complementary sequence to the 3′-end of the polynucleotide sequence to be extended comprised in said one or more polynucleotide or wherein the 3′-terminus of the complement of said polynucleotide primer contains a complementary sequence to the 3′-end of the complement of the polynucleotide sequence to be extended comprised in said one or more polynucleotide, respectively, to yield one hybrid linear polynucleotide having a sequence comprising the polynucleotide sequence to be extended spliced at its 3′-terminus or at its 5′-terminus, respectively, with the polynucleotide sequence of the suitable primer,

b) optionally subjecting the hybrid linear polynucleotide obtained in step a) and another suitable primer to a second overlap-extension PCR reaction, wherein the 3′-terminus of said polynucleotide primer contains a complementary sequence to the 3′-end of the hybrid linear polynucleotide having a sequence comprising the polynucleotide sequence to be extended spliced at its 3′-terminus or at its 5′-terminus, respectively, with the polynucleotide sequence of the suitable primer, or wherein the 3′-terminus of the complement of said polynucleotide primer contains a complementary sequence to the 3′-end of the complement of the hybrid linear polynucleotide having a sequence comprising the polynucleotide sequence to be extended spliced at its 3′-terminus or at its 5′-terminus, respectively, with the polynucleotide sequence of the other suitable primer, respectively, to yield one hybrid linear polynucleotide having a sequence comprising the polynucleotide sequence to be extended spliced at its 3′-terminus and at its 5′-terminus, respectively, with the polynucleotide sequences of the suitable primers,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably the circular vector comprising one or more functional guide-polynucleotide expression cassettes.

In a preferred embodiment according to the method of the third or forth aspect of the invention the assembly reaction occurs in a host cell belonging to S. cerevisiae species.

In one embodiment of the method according to the third or forth aspect the second host cell is a prokaryotic, such as a bacterial host cell, or an eukaryotic host cell, preferably a fungal host cell. E. g. the second host cell is a prokaryotic host cell selected from Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. Alternatively, the second host cell is a fungal host cell, more preferably a filamentous fungal host cell, most preferably a filamentous fungal host cell selected from Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. In yet another embodiment the fungal host cell is a yeast host cell, preferably a yeast host cell selected from Candida, Hansenula, Issatchenkia, Kluyveromyces, Pichia, Saccharomyces, Schizosaccharomyces, Yarrowia or Zygosaccharomyces.

According to one embodiment of any one of the aspects according to the invention the circular vector comprising one or more functional guide polynucleotides expression cassettes comprises at least 2 or more functional guide polynucleotide expression cassettes, preferably at least 3, 4, 5, 6 or more functional guide polynucleotide expression cassettes.

In a fifth aspect, the invention provides a vector, preferably the circular vector comprising one or more functional guide polynucleotide expression cassettes obtainable by the method of according to the first or second aspect of the invention. Any embodiment relating to the previous aspects of the invention is equally applicable to the fifth aspect of the invention.

In the context of all embodiments of the present invention, a vector may be any vector (e.g., a plasmid or virus), which can conveniently be subjected to recombinant DNA procedures and can mediate expression of the one or more guide polynucleotide expression cassettes. The choice of the vector will typically depend on the compatibility of the vector with the host cell into which the vector is to be introduced. Preferred vectors are the vectors used in the examples herein. A vector according to the fifth aspect of the invention is preferably a closed circular plasmid. In other contexts a vector may either be a circular or may be a linear polynucleotide. A vector may be an autonomously replicating vector, i.e., a vector, which exists as an extra-chromosomal entity, the replication of which is independent of chromosomal replication, e.g., a plasmid, an extra-chromosomal element, a mini-chromosome, or an artificial chromosome.

Preferably, the vector is an autonomously replicating vector, preferably a 2p vector (for yeast) or an AMA-vector (for filamentous fungi). An AMA-vector preferably comprise the AMA1-sequence (see e.g. Aleksenko and Clutterbuck 1997) or a functional variant or equivalent thereof.

A vector according to the fifth aspect of the invention is preferably not integrated into the genome of the host cell. Preferably the vector according to the forth aspect is not an integrative vector. A vector according to the fourth aspect of the invention may contain one or more selectable markers, which permit easy selection of transformed cells. The vector comprising one or more guide-polynucleotide expression cassettes, assembled in vivo according to the methods of the invention is preferably an autonomously replicating circular vector. In alternative said vector comprising one or more guide polynucleotide expression cassettes may be an autonomously replicating linear vector. Autonomously replicanting linear vectors are known to those skilled in the art. (Finbarr Hayes (2003); Takahashi S et al (2011); Burke D T, et al (1987)).

In a sixth aspect the invention provides a recombinant host cell comprising a vector, preferably a circular vector comprising one or more functional guide polynucleotides expression cassettes which recombinant host cell is obtainable by the methods according to the third or forth aspect. Any embodiment relating to the previous aspects of the invention is equally applicable to the sixth aspect of the invention.

A recombinant host cell comprising a vector, preferably a circular vector comprising one or more functional guide polynucleotides expression cassettes may be any host cell as defined herein e.g. in the general definitions. When the host cell is a host cell wherein the assembly of the vector, preferably the circular vector occurs, said host cell is preferably a S. cerevisiae host cell. A preferred host cell is a modified host cell wherein expression of a component associated with non-homologous end joining (NHEJ) is altered compared to the corresponding wild-type host cell; preferably expression of the component associated with NHEJ is lowered. Preferred components associated with NHEJ are the yeast Ku70 and Ku80 and their respective orthologs in preferred non-mammalian host cells according to the present invention. Another preferred component associated with NHEJ is the yeast LIG4 and its respective orthologs in preferred non-mammalian host cells according to the present invention.

In a seventh aspect the invention provides with the use of a vector, preferably a circular vector according to the fifth aspect or of a host cell comprising a vector, preferably a circular vector according to the sixth aspect in a CRISP-CAS mediated transformation.

In a eight aspect the invention provides a non-naturally occurring or engineered composition comprising a source of a CRISPR-Cas system comprising a guide-polynucleotide and a Cas protein, wherein the guide-polynucleotide comprises a guide-sequence that essentially is the reverse complement of a target-polynucleotide in a host cell and the guide-polynucleotide can direct binding of the Cas protein at the target-polynucleotide in the host cell to form a CRISPR-Cas complex, wherein the guide-sequence is essentially the reverse complement of the (N)y part of a 5′-(N)yPAM-3′ polynucleotide sequence target in the genome of the host cell, wherein y is an integer of 8-30, wherein PAM is a protospacer adjacent motif, wherein the host cell is preferably a prokaryote or a eukaryote, which eukaryote is a filamentous fungus, preferably an Aspergillus, a Penicillium or a Rasamsonia; or a yeast, preferably a Yarrowia, a Saccharomyces or a Kluyveromyces; Pichia, Issatchenkia or a Mortierella and wherein PAM is preferably a sequence selected from the group consisting of 5′-XGG-3′, 5′-XGGXG-3′, 5′-XXAGAAW-3′, 5′-XXXXGATT-3′, 5′-XXAGAA-3′, 5′-XAAAAC-3′, wherein X can be any nucleotide or analog thereof, preferably X can be any nucleotide; and W is A or T, wherein the guide-polynucleotide is comprised in a vector, preferably a circular vector according to the fifth aspect.

The invention provides a non-naturally occurring or engineered composition comprising a source of a CRISPR-Cas system comprising a guide-polynucleotide and a Cas protein, wherein the guide-polynucleotide comprises a guide-sequence that essentially is the reverse complement of a target-polynucleotide in a host cell and the guide-polynucleotide can direct binding of the Cas protein at the target-polynucleotide in the host cell to form a CRISPR-Cas complex, wherein the guide-sequence is essentially the reverse complement of the (N)y part of a 5′-(N)yPAM-3′ polynucleotide sequence target in the genome of the host cell, wherein y is an integer of 8-30, more preferably 10-30, more preferably 15-30, more preferably 17-27, more preferably 17-20, more preferably 17, 18, 19, 20, 21, 22, 23, 24, 25, 26, or 27, wherein PAM is a protospacer adjacent motif, wherein the host cell is a prokaryote or a eukaryote, which eukaryote is a filamentous fungus, preferably an Aspergillus, a Penicillium or a Rasamsonia or a Mortierella; or a yeast, preferably a Yarrowia, a Saccharomyces or a Kluyveromyces or Pichia or Issatchenkia; and wherein PAM is preferably a sequence selected from the group consisting of 5′-XGG-3′, 5′-XGGXG-3′, 5′-XXAGAAW-3′, 5′-XXXXGATT-3′, 5′-XXAGAA-3′, 5′-XAAAAC-3′, wherein X can be any nucleotide or analog thereof, preferably X can be any nucleotide; and W is A or T, wherein the guide-polynucleotide is comprised in a vector, preferably a circular vector according to the fifth aspect.

The composition, source, CRISPR-Cas system, guide-polynucleotide, Cas protein, target-polynucleotide, host cell and CRISPR-Cas complex are herein referred to as a composition, source, CRISPR-Cas system, guide-polynucleotide, Cas protein, target-polynucleotide, host cell and CRISPR-Cas complex according to the present invention. For the sake of completeness, since “a” is defined elsewhere herein as “at least one”, a composition according to the present invention comprises a source of at least one, i.e. one, two, three or more guide-polynucleotides and/or at least one, i.e. one, two, three or more Cas proteins. Accordingly, the present invention conveniently provides for a multiplex CRISPR-Cas system. Such multiplex CRISPR-Cas system can conveniently be used for introduction of a donor polynucleotide, deletion of a polynucleotide and polynucleotide library insertion into the genome of a host cell.

The term “non-naturally occurring composition” refers in the context of all embodiments of the present invention to a composition that in its form used in the present invention does not occur in nature. The individual elements may e.g. occur as such or in combinations with other elements in nature, but the non-naturally occurring composition comprises e.g. at least one element more or less than a naturally composition.

The term “engineered composition” refers in the context of all embodiments of the present invention to a composition wherein at least one of the elements has been engineered, i.e. modified by man, in such a way that resulting element does not occur in nature. It follows that by virtue of comprising at least one engineered element, an engineered composition does not occur in nature.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence-specific manner. The complex may comprise two strands forming a duplex structure, three or more strands forming a multi stranded complex, a single self hybridizing strand, or any combination of these. A hybridization reaction may constitute a step in a more extensive process, such as the cleavage of a polynucleotide by an enzyme. Preferred hybridization conditions are physiological conditions as within a host cell according to the present invention.

The term “source” in the context of all embodiments of the present invention refers to any source of a CRISPR-Cas system comprising a guide-polynucleotide and a Cas protein. The guide-polynucleotide and Cas protein may be present in separate sources. In such case, the composition according to the present invention comprises a CRISPR-Cas system comprising a source of a guide-polynucleotide and a source of a Cas-protein. Any source means that the guide-polynucleotide and Cas protein may be present as such in a form that they can function within a CRISPR-Cas system. The guide-polynucleotide and/or the Cas-protein may be provided in its active forms and may e.g. be provided from an inactive form or from another entity. The guide-polynucleotide may e.g. be present on another polynucleotide or may be encoded by a polynucleotide that is transcribed to provide for the actual guide-polynucleotide. The Cas protein may be encoded by a polynucleotide (e.g. DNA or mRNA) that is transcribed and/or translated to provide the actual Cas protein. An encoding polynucleotide may be present in a nucleic acid construct as defined herein and/or in a vector as defined herein. Such nucleic acid construct and vector are herein referred to as a nucleic acid construct according to the present invention and a vector according to the present invention.

Preferably, in the composition according to the present invention, the Cas protein is encoded by a polynucleotide and/or the guide-polynucleotide is encoded by or present on a polynucleotide.

Preferably, in the composition according to the present invention, the Cas protein is encoded by a polynucleotide and/or the guide-polynucleotide is encoded by or present on another polynucleotide and the polynucleotide or polynucleotides are comprised in a vector.

Preferably, in the composition according to the present invention, the Cas protein is encoded by a polynucleotide and the guide-polynucleotide is encoded by or present on another polynucleotide and the polynucleotides are comprised in one vector.

Preferably, in the composition according to the present invention, the Cas protein is encoded by a polynucleotide comprised in a vector and the guide-polynucleotide is encoded by or present on another polynucleotide comprised in another vector.

The invention thus provides for the possibilities that the guide-polynucleotide and the Cas protein are provided as such, or that they are encoded on or present on a vector, such as the vector according to the fifth aspect of the invention. In the latter case, the encoding polynucleotides may each be on a separate vector or may both be on a single vector.

The amount of Cas protein in a source in a composition according to the present invention may vary and may be optimized for optimal performance. It may be convenient to avoid too high levels of Cas protein in a host cell since high levels of Cas protein may be toxic to the host cell, even without a guide-polynucleotide present (see e.g. Ryan et al 2014 and Jacobs et al., 2014). A person skilled in the art knows how to regulate expression levels, such as by choosing a weaker promoter or inducible promoter for expression of the Cas protein. Examples of promoters suitable for expression of a protein are depicted elsewhere herein.

In a composition according to the present invention wherein a guide-polynucleotide according to the present invention is encoded by a polynucleotide, expression of the guide-polynucleotide may be facilitated by a promoter operably linked to the encoding polynucleotide. Such promoter may be any suitable promoter known to the person skilled in the art. Several types of promoters can be used. It may be convenient to use an RNA polymerase III promoter or an RNA polymerase II promoter. Background information on RNA polymerase III and its promoters can be found e.g. in Marck et al., 2006. In some cases, such as in S. cerevisiae, S. pombe, RNA polymerase III promoters include promoter elements in the transcribed region. Accordingly, it may be convenient to use an RNA polymerase II promoter; these are known to the person skilled in the art and reviewed in e.g. Kornberg 1999. However, transcripts from an RNA II polymerase often have complex transcription terminators and transcripts are polyadenylated; this may hamper with the requirements of the guide-polynucleotide which because both its 5′ and 3′ ends need to be precisely defined in order to achieve the required secondary structure to produce a functional CRISPR-Cas system. These drawbacks can however be circumvented. In case an RNA polymerase II promoter is used, the polynucleotide encoding the guide-polynucleotide may also encode self-processing ribozymes and may be operably linked to an RNA polymerase II promoter; as such the polynucleotide encodes a pre-guide-polynucleotide comprising the guide-polynucleotide and self-processing ribozymes, wherein, when transcribed, the guide-polynucleotide is released by the self-processing ribozymes from the pre-guide-polynucleotide transcript. Preferred constructs comprising a polynucleotide encoding a pre-guide-polynucleotide according to the present invention operably linked to an RNA polymerase II promoter are those depicted in examples 8-17 and 18-34 herein. Background information on such constructs can be found in e.g. Gao et al, 2014 et al.

Preferably, in a composition according to the present invention wherein the guide-polynucleotide is encoded by a polynucleotide, said polynucleotide is operably linked to an H1 RNA polymerase III promoter, preferably a human H1 RNA polymerase III promoter.

Preferably, in a composition according to the present invention wherein the guide-polynucleotide is encoded by a polynucleotide, said polynucleotide is operably linked to a U6 RNA polymerase III promoter, preferably a human U6 RNA polymerase III promoter.

Preferably, in a composition according to the present invention wherein the guide-polynucleotide is encoded by a polynucleotide, said polynucleotide is operably linked to an SNR52p RNA polymerase III promoter, preferably a yeast SNR52p RNA polymerase III promoter. Such promoter is preferably used when the host is a yeast host cell, such as a Saccharomyces or a Kluyveromyces.

Preferably, in a composition according to the present invention wherein the guide-polynucleotide is encoded by a polynucleotide, said polynucleotide is operably linked to an RNA polymerase II promoter and encodes a pre-guide-polynucleotide comprising the guide-polynucleotide and self-processing ribozymes, wherein, when transcribed, the guide-polynucleotide is released by the self-processing ribozymes from the pre-guide-polynucleotide transcript.

The composition according to the eight aspect of the present invention can conveniently be used to modulate expression of a polynucleotide in a host cell. Accordingly, in a ninth aspect, the present invention provides a method of modulating expression of a polynucleotide in a host cell, comprising contacting a host cell with the composition according to the eight aspect of the invention, wherein the guide-polynucleotide directs binding of the Cas protein at the target-polynucleotide in the host cell to form a CRISPR-Cas complex, preferably wherein the host cell comprises a polynucleotide encoding a compound of interest.

The term “expression” in the context of the present invention is herein defined as the process by which a polynucleotide is transcribed from a polynucleotide template (e.g. a DNA template polynucleotide is transcribed into an mRNA polynucleotide transcript or other RNA transcript) and/or the process by which an mRNA transcript is subsequently translated into peptides, polypeptides, or proteins. Transcripts and encoded polypeptides may be collectively referred to as “gene product”. If the polynucleotide transcript is derived from a genomic template DNA, expression may include splicing of the mRNA transcript in a host cell. The term “modulating expression” refers herein to increased or reduced expression compared to a parent host cell wherein expressing is not modulated when assayed using the same conditions. Reduced expression may be a reduced amount of transcript such as mRNA and/or a reduced amount of translation product such as a polypeptide. It follows that increased expression may be an enhanced amount of transcript such as mRNA and/or an enhanced amount of translation product such as a polypeptide.

Preferably, the CRISPR-Cas complex cleaves one or both polynucleotide strands at the location of the target-polynucleotide, resulting in modulated expression of the gene product. The CRISPR-Cas complex may also have altered nuclease activity and substantially lack the ability to cleave one or both strands of a target-polynucleotide; in such case, expression is modulated by the binding of the complex to the target-polynucleotide. A Cas protein lacking substantially all enzyme activity can conveniently be used for gene silencing or down regulation of expression since the CRISPR-Cas complex will hamper transcription from the target-polynucleotide. Alternatively, a Cas protein can be modified into a transcription factor for programmable transcriptional activation or silencing of a gene of interest (Larson, et al., 2013).

A composition according to the eight aspect of the present invention can conveniently be used for the deletion of polynucleotide. In an embodiment, when the composition according to the eight aspect of the present invention comprises a source of at least one or two guide-polynucleotides and/or a source of at least at least one Cas protein, at least one CRISPR-Cas complex or two different CRISPR-Cas complexes are formed that cleave one or both polynucleotide strands at one location or at different locations of the target-polynucleotide, resulting in deletion of a polynucleotide fragment from the target-polynucleotide. Preferably, such composition according to the present invention comprising at least one or two guide-polynucleotides and/or a source of at least at least one Cas protein, additionally comprises an exogenous polynucleotide as defined herein below that is at least partly complementary to the at least one or two target-polynucleotides targeted by the guide-polynucleotide(s). Such polynucleotide fragment to be deleted or deleted fragment may be several nucleotides in length up to a few thousand nucleotides in length, an entire gene may be deleted or a cluster of genes may be deleted. Accordingly, the present invention provides for a method of modulating expression of a polynucleotide in a host cell, wherein a polynucleotide fragment is deleted from a target-polynucleotide.

In an embodiment, the method of modulating expression comprises cleavage of one or both polynucleotide strands at least one location of the target-polynucleotide followed by modification of the target-polynucleotide by homologous recombination with an exogenous polynucleotide. In such case, the composition according to the eight aspect of the present invention preferably further comprises such exogenous polynucleotide. Such modification may result in insertion, deletion or substitution of at least one nucleotide in the target-polynucleotide, wherein the insertion or substitution nucleotide may originate from the exogenous polynucleotide. A modification can also be made when the exogenous polynucleotide is a non-integrating entity such as described in Dong et al., and Beetham et al.; in this case the target-polynucleotide is modified but no nucleotide of the exogenous polynucleotide is introduced into the target-polynucleotide. Consequently, the resulting host is a non-recombinant host cell when the Cas-protein according to the invention is transformed as a protein. The exogenous polynucleotide may be any polynucleotide of interest such as a polynucleotide encoding a compound of interest as defined herein below, or a part of such polynucleotide or a variant thereof. Such exogenous polynucleotide is herein referred to as an exogenous polynucleotide according to the present invention and may single-stranded or double-stranded.

Various applications can be considered by the person skilled in the art for the compositions and methods according to the present invention. A polynucleotide (or gene) in a genome may be modified, edited or disrupted using compositions and methods according to the present invention. E.g. when a fully active Cas protein is used that cuts in both strands of the target-polynucleotide and when no exogenous polynucleotide is present as a suitable repair template, the double strand break is repaired by non-homologous end joining repair (NHEJ). During NHEJ insertions and/or deletions (which may be construed as substitution in some cases) of one or several nucleotides may occur, these are randomly inserted or deleted at the repair site; this is characteristic for NHEJ. Such insertions and/or deletions may impact the reading frame of the coding sequence, resulting amino acid changes in the gene product or even a truncated protein in case of genesis of a (premature) stop codon or alteration of a splice site.

A polynucleotide (or gene) in a genome may be modified, edited or disrupted using compositions and methods according to the present invention using homologous end joining repair (HEJ), also known as homology-directed repair (HDR), when an exogenous polynucleotide is present as repair template. E.g. when an exogenous polynucleotide having sequence identity to the target-polynucleotide (i.e. upstream (5′) and downstream (3′) of the double strand break) is present together with a CRISPR-Cas system according to the present invention, HDR will introduce (or actually reproduce) the corresponding nucleotides of the exogenous polynucleotide at the double strand break in the target-polynucleotide. Preferably, an exogenous polynucleotide according to the present invention does not contain the target sequence itself followed by a functional PAM sequence to avoid the risk of the exogenous polynucleotide itself or the modified target-polynucleotide being (re)cut by the CRISPR-CAS system.

In the embodiments of the present invention, when a CRISPR-Cas system according to the present invention comprises an exogenous polynucleotide (donor polynucleotide, donor DNA, repair template), the CRISPR-Cas system according to the present invention preferably comprises two or more guide-polynucleotides encoded by or present on one or more separate polynucleotides or vectors, and two or more exogenous polynucleotides are provided together with said CRISPR-Cas system enabling the formation of two or more CRISPR-CAS complexes. In a method according to the present invention, such CRISPR-Cas systems according to the present invention can conveniently be used to modulate expression at two or more target-polynucleotides, i.e. a method to target multiple target sites. Such CRISPR-Cas system according to the present invention will by chance form one, two or more CRISPR-CAS complexes at one or more target-polynucleotides. Such method can be used to generate one or more insertions, deletions, substitutions, optionally in combination with the one or more exogenous polynucleotides, in the genome of the host cell, or to modulate expression of genes via the formed CRISPR-CAS complexes.

Preferably, the CRISPR-Cas complex cleaves one or both polynucleotide strands at the location of the target-polynucleotide, resulting in modulated expression of the gene product. The CRISPR-Cas complex may also have altered nuclease activity and lack the ability to cleave one or both strands of a target-polynucleotide; in such case, expression is modulated by the binding of the complex to the target-polynucleotide.

In an embodiment, when the composition according to the eight aspect of the present invention comprises a source of at least one or two guide-polynucleotides and/or a source of at least one Cas protein, at least one CRISPR-Cas complex or two different CRISPR-CAS complexes are formed that cleave one or both polynucleotide strands at one location or at different locations of the target-polynucleotide, resulting in deletion of a polynucleotide fragment from the target-polynucleotide. Preferably, such composition according to the present invention comprising at least one or two guide-polynucleotides and/or a source of at least at least one Cas protein, additionally comprises an exogenous polynucleotide as defined herein below that is at least partly complementary to the at least one or two target-polynucleotides targeted by the guide-polynucleotide(s). Such polynucleotide fragment to be deleted or deleted fragment may be from several nucleotides in length up to a few thousand nucleotides in length, an entire gene may be deleted or a cluster of genes may be deleted. Accordingly, the present invention provides for a method of modulating expression of a polynucleotide in a host cell, wherein a polynucleotide fragment is deleted from a target-polynucleotide.

In one embodiment a method of modulating expression of a polynucleotide in a host cell, wherein a polynucleotide fragments is deleted from a target-polynucleotide, comprises contacting a host cell with a composition as descried herein, wherein the guide-polynucleotide directs binding of the Cas protein at the target-polynucleotide in the host cell to form a CRISPR-Cas complex. Preferably a method of modulating expression of a polynucleotide in a host cell, wherein a polynucleotide fragments is deleted from a target-polynucleotide, comprises contacting a host cell with a composition as descried herein, wherein the guide-polynucleotide directs binding of the Cas protein at the target-polynucleotide in the host cell to form a CRISPR-Cas complex, wherein the host cell is a modified host cell deficient in a component associated with NHEJ. In another preferred embodiment a method of modulating expression of a polynucleotide in a host cell, wherein a polynucleotide fragments is deleted from a target-polynucleotide, comprises contacting a host cell with a composition as descried herein, wherein the guide-polynucleotide directs binding of the Cas protein at the target-polynucleotide in the host cell to form a CRISPR-Cas complex, wherein the host cell is a modified host cell deficient in a component associated with NHEJ, wherein the composition as described herein does not comprise an exogenous or donor polynucleotide. In one preferred embodiment the component associated with NHEJ is a yeast Ku70 or a yeast Ku80 or a yeast LIG4 or its respective ortholog in the host cells according to the present invention. In another embodiment the host cell is a filamentous fungal host cell. In another embodiment of the method of modulating expression of a polynucleotide in a host cell the composition is comprised in an AMA vector.

Therefore the present invention relates in one embodiment to a method of modulating expression of a polynucleotide in a cell, wherein a polynucleotide fragment is deleted from a target-polynucleotide, comprising contacting a host cell with the composition as described herein but preferably not comprising a donor polynucleotide as defined herein, wherein the guide-polynucleotide directs binding of the Cas protein at the target-polynucleotide in the host cell to form a CRISPR-Cas complex, wherein the host cell is deficient in a component associated with NHEJ, preferably a yeast Ku70 or yeast Ku80 or a yeast LIG4 or its respective ortholog in the host cells.

Surprisingly it has been found that in a host cell deficient in a gene involved in NHEJ it is possible to obtain deletions in the host cell genome in a controlled way by using the CRISPR/CAS9 system when regions of homology are present at both sites of the intended cleavage site and wherein the composition as described herein does not comprise a donor DNA, in a method of modulating expression of a polynucleotide in a cell, wherein a polynucleotide fragment is deleted from a target-polynucleotide, as described herein.

Therefore in one embodiment the invention relates to a method of modulating expression of a polynucleotide in a cell, wherein a polynucleotide fragment is deleted from a target-polynucleotide, comprising contacting a host cell with a non-naturally occurring or engineered composition comprising a source of a CRISPR-Cas system comprising a guide-polynucleotide and a Cas protein, wherein the guide-polynucleotide comprises a guide-sequence that essentially is the reverse complement of a target-polynucleotide in a host cell and the guide-polynucleotide can direct binding of the Cas protein at the target-polynucleotide in the host cell to form a CRISPR-Cas complex, wherein the guide-sequence is essentially the reverse complement of the (N)y part of a 5′-(N)yPAM-3′ polynucleotide sequence target in the genome of the host cell, wherein y is an integer of 8-30, wherein PAM is a protospacer adjacent motif, wherein the host cell is a eukaryote, which eukaryote is a filamentous fungus, preferably an Aspergillus, a Penicillium, a Rasamsonia or a Mortierella and wherein PAM is preferably a sequence selected from the group consisting of 5′-XGG-3′, 5′-XGGXG-3′, 5′-XXAGAAW-3′, 5′-XXXXGATT-3′, 5′-XXAGAA-3′, 5′-XAAAAC-3′, wherein X can be any nucleotide or analog thereof, preferably X can be any nucleotide; and W is A or T herein but preferably not comprising a donor polynucleotide as defined herein, wherein the guide-polynucleotide directs binding of the Cas protein at the target-polynucleotide in the host cell to form a CRISPR-Cas complex, wherein the host cell is deficient in a component associated with NHEJ, preferably a yeast Ku70 or yeast Ku80 or a yeast LIG4 or its respective ortholog in the host cells, wherein the Cas protein has activity for directing cleavage of both polynucleotide strands at the location of the target-sequence and wherein the cleavage occurs in a region of the genome comprised between two homologous regions which upon cleavage by the Cas protein recombine with each other resulting in the deletion of a polynucleotide comprised between said regions. Preferably the degree of homology between the two homologous regions is such to allow homologous recombination. Preferably the two homologous regions have at least 60%, 70%, 80%, 90%, 99% or 100% sequence identity over the whole length of the homologous regions. It has been surprisingly found that the length of homologous region can be very short even in filamentous fungi, wherein usually a length of at least 1 or several kb is necessary to allow homologous recombination. Therefore in a preferred embodiment the length of the homologous regions is preferably at most 1 kb, at most 0.5 kb, at most 100 bp, at most 50 bp, at most 40 bp, at most 30 bp, at most 20 bp, at most 10 bp.

Preferably the distance between the two homologous regions is at most 10 kb, at most 9, at most 8 kb, at most 7 kb, at most 6 kb, at most 5 kb, at most 4 kb, at most 3 kb, at most 2 kb, at most 1 kb, at most 0.5 kb, at most 100 bp, at most 50 bp, at most 40 bp, at most 30, 20, 10 kb.

In one aspect, the invention relates to a software algorithms able to identify PAM sites in the genome comprised between homology regions of about 7-20 bp in a neighborhood of the PAM site to design a method to target one or more PAM sites and create deletion of polynucleotides without use of a donor DNA.

The above method can be used for efficient removal of polynucleotide sequences in a designed way. For example upon introducing a Cas9 expression cassette at the genomic DNA and after several rounds of modifications mediated by the CRISPR/CAS9 system, one can remove the CAS9 from the genome by the introduction of a gRNA targeting a site in the Cas9 expression casette and wherein the Cas9 expression cassette is comprised between two homologous regions as defined above, preferably 100-bp long, more preferably 20-bp, 15-bp long or shorter and cleave out the Cas9 open reading frame or a large part of the expression cassette.

The above method can also be used for transient inactivation of a gene. Eg. one could for example make a gene, e.g. a Ku70 polynucleotide non-functional by inserting a polynucleotide sequence in the ORF of the Ku70 gene, comprising two homologous regions at its 5′-end and 3′end respectively, wherein preferably the homologous regions are 100-bp, more preferably 20-bp, 15-bp long or shorter. The Ku70 gene can be made functional again using a CRISPR-Cas9 system without donor DNA as described above.

In an embodiment, the method of modulating expression comprises cleavage of one or both polynucleotide strands at at least one location of the target-polynucleotide followed by modification of the target-polynucleotide by homologous recombination with an exogenous polynucleotide. In such case, the composition according to the first aspect of the present invention preferably further comprises such exogenous polynucleotide. Such modification may result in insertion, deletion or substitution of at least one nucleotide in the target-polynucleotide, wherein the insertion or substitution nucleotide may or may not originate from the exogenous polynucleotide. In one embodiment the exogenous polynucleotide comprises regions of homology with the target-polynucleotide. Preferably the degree of homology between these homologous regions is such to allow homologous recombination. Preferably the homologous regions have at least 60%, 70%, 80%, 90%, 99% or 100% sequence identity over the whole length of the homologous regions. In one embodiment, wherein the host cell is deficient in a component involve in NHEJ as defined herewith, the homologous regions are preferably at most 1 kb, at most 0.5 kb, at most 100 bp, at most 50 bp, at most 40 bp, at most 30 bp, at most 20 bp, at most 10 bp.

A modification can also be made when the exogenous polynucleotide is a non-integrating entity; in this case the target-polynucleotide is modified but no nucleotide of the exogenous polynucleotide is introduced into the target-polynucleotide. Consequently, the resulting host is a non-recombinant host when the Cas-protein according to the present invention is transformed as a protein. In a method according to this aspect of the present invention, the host cell may thus be a recombinant host cell or may be a non-recombinant host cell. The exogenous polynucleotide may be any polynucleotide of interest such as a polynucleotide encoding a compound of interest as defined herein, or a part of such polynucleotide or a variant thereof.

In a further aspect, the present invention provides a method of producing a host cell, comprising contacting a host cell with the composition according to the eight aspect of the present invention, wherein the guide-polynucleotide directs binding of the Cas protein at the target-polynucleotide in the host cell to form a CRISPR-Cas complex. A host cell in this embodiment of the present invention may be any type of host cell as defined herein and may comprise a polynucleotide encoding a compound of interest as defined elsewhere herein. A preferred method of producing a host cell according to the present invention comprises a step to produce an offspring host cell, wherein in said offspring host cell no components of a CRISPR-Cas system according to the present invention are present anymore. A further preferred host cell is a modified host cell wherein expression of a component associated with NHEJ as depicted above is altered compared to the corresponding wild-type host cell; preferably expression of the component associated with NHEJ is lowered.

The composition according to the eight aspect of the present invention may be any such composition as defined herein. Contacting a host cell with a composition according to the present invention may be performed by any means known to the person skilled in the art. A host cell according to the present invention may simply be brought into a solution comprising a composition according to the present invention. Specific means of delivering a composition according to the present invention into a host cell may be used.

In a tenth aspect, the present invention provides for a method for the production of a compound of interest, comprising culturing under conditions conducive to the compound of interest a host cell according to the sixth aspect of the present invention or a host cell obtainable or obtained by a method according to the third or forth aspect of the present invention, and optionally purifying or isolating the compound of interest.

A compound of interest in the context of all embodiments of the present invention may be any biological compound. The biological compound may be biomass or a biopolymer or a metabolite. The biological compound may be encoded by a single polynucleotide or a series of polynucleotides composing a biosynthetic or metabolic pathway or may be the direct result of the product of a single polynucleotide or products of a series of polynucleotides, the polynucleotide may be a gene, the series of polynucleotide may be a gene cluster. In all embodiments of the present invention, the single polynucleotide or series of polynucleotides encoding the biological compound of interest or the biosynthetic or metabolic pathway associated with the biological compound of interest, are preferred targets for the compositions and methods according to the present invention. The biological compound may be native to the host cell or heterologous to the host cell.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. The term polypeptide refers to polymers of amino acids of any length. The polymer may he linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. Polypeptides further include naturally occurring allelic and engineered variations of the above-mentioned polypeptides and hybrid polypeptides. The polypeptide may be native or may be heterologous to the host cell. The polypeptide may be a collagen or gelatine, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may also be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase, monooxygenase.

According to the present invention, a compound of interest can be a polypeptide or enzyme with improved secretion features as described in WO2010/102982. According to the present invention, a compound of interest can be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter(s) and terminator. The hybrid polypeptides may comprise a combination of partial or complete polypeptide sequences obtained from at least two different polypeptides wherein one or more may be heterologous to the host cell. Example of fusion polypeptides and signal sequence fusions are for example as described in WO2010/121933.

The biopolymer may be a polysaccharide. The polysaccharide may be any polysaccharide, including, but not limited to, a mucopolysaccharide (e. g., heparin and hyaluronic acid) and nitrogen-containing polysaccharide (e.g., chitin). In a preferred option, the polysaccharide is hyaluronic acid.

A polynucleotide coding for the compound of interest or coding for a compound involved in the production of the compound of interest according to the invention may encode an enzyme involved in the synthesis of a primary or secondary metabolite, such as organic acids, carotenoids, (beta-lactam) antibiotics, and vitamins. Such metabolite may be considered as a biological compound according to the present invention.

The term “metabolite” encompasses both primary and secondary metabolites; the metabolite may be any metabolite. Preferred metabolites are citric acid, gluconic acid, adipic acid, fumaric acid, itaconic acid and succinic acid.

A metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

A primary metabolite may be, but is not limited to, an amino acid, fatty acid, nucleoside, nucleotide, sugar, triglyceride, or vitamin.

A secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof.

According to the invention, a compound of interest is preferably a polypeptide as described in the list of compounds of interest.

According to another embodiment of the invention, a compound of interest is preferably a metabolite.

The host cell according to the present invention may already be capable of producing the compound of interest. The mutant microbial host cell may also be provided with a homologous or heterologous nucleic acid construct that encodes a polypeptide wherein the polypeptide may be the compound of interest or a polypeptide involved in the production of the compound of interest. The person skilled in the art knows how to modify a microbial host cell such that it is capable of producing the compound of interest

More than one copy of a polynucleotide according to the present invention may be inserted into the microbial host cell to mediate production of the product encoded by said polynucleotide. This can be done, preferably by integrating multiple copies of the polynucleotide into the genome of the host cell, more preferably by targeting the integration of the polynucleotide at one of the highly expressed loci defined in the former paragraph. Alternatively, integration of multiple copies can be achieved by including an amplifiable selectable marker gene with a polynucleotide according to the present invention, such that cells containing amplified copies of the selectable marker gene (and thereby additional copies of the nucleic acid sequence) can be selected for by cultivating the cells in the presence of the appropriate selectable agent. To increase the number of copies of a polynucleotide according the present invention even more, the technique of gene conversion as described in WO98/46772 may be used.

### General Definitions

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element or more than one element.

The word “about” or “approximately” when used in association with a numerical value (e.g. about 10) preferably means that the value may be the given value (of 10) more or less 1% of the value.

A preferred nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage. A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the present invention of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Within the present invention, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gin or his; Asp to glu; Cys to ser or ala; Gin to asn; Glu to asp; Gly to pro; His to asn or gin; lie to leu or val; Leu to ile or val; Lys to arg; gin or glu; Met to leu or ile; Phe to met, leu or tyr; Ser to thr; Thr to ser; Trp to tyr; Tyr to trp or phe; and, Val to ile or leu.

A polynucleotide according to the present invention is represented by a nucleotide sequence. A polypeptide according to the present invention is represented by an amino acid sequence. A nucleic acid construct according to the present invention is defined as a polynucleotide which is isolated from a naturally occurring gene or which has been modified to contain segments of polynucleotides which are combined or juxtaposed in a manner which would not otherwise exist in nature. Optionally, a polynucleotide present in a nucleic acid construct according to the present invention is operably linked to one or more control sequences, which direct the production or expression of the encoded product in a host cell or in a cell-free system.

The sequence information as provided herein should not be so narrowly construed as to require inclusion of erroneously identified bases. The skilled person is capable of identifying such erroneously identified bases and knows how to correct for such errors.

All embodiments of the present invention, i.e. a composition according to the present invention, a method of modulating expression, a host cell comprising a composition according to the present invention, a method of producing a host cell according to the present invention, a host cell according to the present invention and a method for the production of a compound of interest according to the present invention preferably refer to host cell, not to a cell-free in vitro system; in other words, the CRISPR-Cas systems according to the present invention are preferably host cell systems, not cell-free in vitro systems.

In all embodiments of the present invention, e.g. a composition according to the present invention, a method of modulating expression, a host cell comprising a composition according to the present invention, a method of producing a host cell according to the present invention, a host cell according to the present invention and a method for the production of a compound of interest according to the present invention, the host cell preferably is a microbial cell, which microbial host cell preferably is a prokaryotic or a eukaryotic host cell. When the host cell according to the present invention is a eukaryotic host cell, the host cell may or not be a mammalian host cell; in the latter case the eukaryotic host cell according to the present invention is not a mammalian host cell. A host cell according to the present invention may be a haploid, diploid or polyploid host cell.

The host cell according to the present invention may be a prokaryotic host cell. Preferably, the prokaryotic host cell according to the present invention is a bacterial host cell. The term “bacterial host cell” includes both Gram-negative and Gram-positive microorganisms. Preferably, a bacterial host cell according to the present invention is from a genus selected from the group consisting of Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces. More preferably, the bacterial host cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Paracoccus denitrificans, Escherichia coli, Corynebacterium glutamicum, Staphylococcus carnosus, Streptomyces lividans, Sinorhizobium melioti and Rhizobium radiobacter.

The host cell according to the present invention may be a eukaryotic host cell. Preferably, the eukaryotic host cell according to the present invention is a mammalian, insect, plant, fungal, or algal host cell, more preferably, the eukaryotic host cell according to the present invention is an insect, plant, fungal, or algal host cell, even more preferably, the eukaryotic host cell according to the present invention is a fungal, or algal host cell such as a Schizochitrium, even more preferably, the eukaryotic host cell according to the present invention is a fungal host cell, even more preferably, the eukaryotic host cell according to the present invention is a filamentous fungal host cell. Preferred mammalian host cells are selected from the group consisting of Chinese hamster ovary (CHO) cells, COS cells, 293 cells, PerC6 cells, and hybridomas. Preferred insect host cells are selected from the group consisting of Sf9 and Sf21 cells and derivatives thereof. A preferred fungal host cell is a yeast host cell, a preferred yeast host cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyces lactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica CLIB122, Yarrowia lipolytica ML324 (deposited as ATCC18943), Pichia stipidis, Scheffersomyces stipitis, and Pichia pastoris. A more preferred fungal host cell is a filamentous fungal cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell according to the present invention is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255(ATCC28089); even more preferably the filamentous fungal host cell according to the present invention is an Aspergillus niger. When the host cell according to the present invention is an Aspergillus niger host cell, the host cell preferably is CBS 513.88, CBS124.903 or a derivative thereof. Several strains of filamentous fungi are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells according to the present invention are Aspergillus niger CBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255(ATCC28089), Penicillium citrinum ATCC 38065, Penicillium chrysogenum P2, Thielavia terrestris NRRL8126, Rasamsonia emersonii CBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC1 1906, Myceliophthora thermophila C1, Garg 27K, VKM-F 3500 D, Chrysosporium lucknowense C1, Garg 27K, VKM-F 3500 D, ATCC44006 and derivatives thereof. Preferably, and more preferably when the microbial host cell according to the invention is a filamentous fungal host cell, a host cell according to the present invention further comprises one or more modifications in its genome such that the host cell is deficient in the production of at least one product selected from glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE if compared to a parent host cell and measured under the same conditions. Oxalic acid hydrolase (oahA) is a component of the synthesis pathway of oxalic acid in many host cells. A host cell deficient in oahA will be deficient in oxalic acid. Oxalic acid is an unwanted by-product in many applications such as food applications. Furthermore, oxalic acid lowers the pH of the medium cultivations of host cell producing this component, resulting in lowered yields; i.e. yield is increased in oxalic acid deficient host cells. It is therefore advantageous if a host cell according to the present invention is deficient in oahA. OahA deficient host cells and preferred methods of producing said host cells are extensively described in WO 2000/50576 and WO2004/070022. A preferred method to produce an oahA deficient host cell is the recombinant method of disruption described in WO 2000/50576. Preferably, a host cell according to the present invention is deficient in oahA. Preferably, the oahA is a fungal oahA. More preferably, the oahA is the oahA from Aspergillus. Even more preferably the oahA is the oahA from Aspergillus niger. Even more preferably the oahA is the oahA from Aspergillus niger CBS 513.88. Most preferably, the oahA comprises the sequence of An10g00820.

prtT is a transcriptional activator of proteases in eukaryotic cells. Several fungal transcriptional activators of proteases have been recently described in WO 00/20596, WO 01/68864, WO 2006/040312 and WO 2007/062936. These transcriptional activators were isolated from Aspergillus niger (A. niger), Aspergillus fumigatus (A. fumigatus), Penicillium chrysogenum (P. chrysogenum) and Aspergillus oryzae (A. oryzae). These transcriptional activators of protease genes can be used to improve a method for producing a polypeptide in a host cell, wherein the polypeptide is sensitive for protease degradation. When a host cell according to the present invention is deficient in prtT, the host cell will produce less proteases that are under transcriptional control of prtT. It is therefore advantageous when a host cell according to the invention is deficient in prtT. prtT deficient hosts and preferred methods to produce these hosts are extensively described in WO 01/68864, WO 2006/040312. WO 01/68864 and WO 2006/040312 describe recombinant and classic methods to disrupt the prtT coding sequence. WO 2007/062936 describes disruption of the prtT binding site in a protease promoter. Disruption of the binding site impedes binding of prtT to the binding site. Consequently, the transcription of the protease is not activated by prtT and less protease is produced.

Preferably, a host cell according to the present invention comprises a polynucleotide encoding prtT, said polynucleotide comprising a modification such that the host cell is deficient in the production of prtT compared to a parent cell it originates from when cultivated under comparable conditions. Preferably, the prtT is a fungal prtT. More preferably, the prtT is the prtT from Aspergillus. Even more preferably the prtT is the prtT from Aspergillus niger. Even more preferably the prtT is the prtT from Aspergillus niger CBS 513.88. Most preferably, the prtT comprises the sequence of An04g06940.

The term “glucoamylase” (glaA) is identical to the term “amyloglucosidase” and is defined herein as an enzyme having dextrin 6-alpha-D-glucanohydrolase activity which catalyses the endo hydrolysis of 1, 6-alpha-D-glucoside linkages at points of branching in chains of 1, 4-linked alpha-D-glucose residues and terminal 1, 4-linked alpha-D-glucose residues. Glucoamylase activity can be measured as AGIU/ml by determining the liberation of paranitrofenol from the substrate p-nitrophenyl-a-D-glucopyranoside (Sigma). This results in a yellow colour, whose absorbance can be measured at 405 nm using a spectrophotometer. 1 AGIU is the quantity of enzyme, which produces 1 μmole of glucose per minute at pH 4.3 and 60° C. from a soluble starch substrate. In WO98/46772 additional details of the assay can be found.

Preferably, a host cell according to the present invention comprises a polynucleotide encoding glaA, said polynucleotide comprising a modification such that the host cell is deficient in the production of glaA compared to a parent cell it originates from when cultivated under comparable conditions. Preferably, the glaA is a fungal glaA. More preferably, the glaA is the glaA from Aspergillus. Even more preferably the glaA is the glaA from Aspergillus niger. Even more preferably the glaA is the glaA from Aspergillus niger CBS 513.88. Most preferably, the glaA comprises the sequence of An03g06550.

The term “alpha-amylase” is defined herein as 1, 4-alpha-D-glucan glucanohydrolase activity which catalyzes the endohydrolysis of polysaccharides with three or more alpha-1, 4-linked glucose units in the presence of water to malto-oligosaccharides. To determine the (neutral) alpha-amylase activity, the Megazyme cereal alpha-amylase kit is used (Megazyme, CERALPHA alpha amylase assay kit, catalogus. ref. K-CERA, year 2000-2001), according a protocol of the supplier. The measured activity is based on hydrolysis of non-reducing-endblocked ρ-nitrophenyl maltoheptaoside in the presence of excess glucoamylase and α-glucosidase at a pH of 7.0. The amount of formed ρ-nitrophenol is a measure for alpha-amylase activity present in a sample.

The term “acid stable alpha-amylase” (amyA) is defined herein as an enzyme having alpha-amylase activity with optimal activity in the acid pH range. To determine the acid stable alpha-amylase activity, also the Megazyme cereal alpha-amylase kit is used (Megazyme, CERALPHA alpha amylase assay kit, catalogus. ref. K-CERA, year 2000-2001), according a protocol of the supplier but at an acid pH. The measured activity is based on hydrolysis of non-reducing-endblocked ρ-nitrophenyl maltoheptaoside in the presence of excess glucoamylase and α-glucosidase at a pH of 4.5. The amount of formed ρ-nitrophenol is a measure for acid stable alpha-amylase activity present in a sample.

Preferably, a host cell according to the present invention comprises a polynucleotide encoding AmyA, said polynucleotide comprising a modification, wherein the host cell is deficient in amyA compared to the parent cell it originates from when cultivated under comparable conditions. Preferably, the amyA is a fungal amyA. More preferably, the amyA is the amyA from Aspergillus. Even more preferably the amyA is the amyA from Aspergillus niger. Even more preferably the amyA is the amyA from Aspergillus niger CBS 513.88. Most preferably, the amyA comprises the sequence of An11g03340.

The term “neutral alpha-amylase activity” (amy) is defined herein as an enzyme having alpha-amylase activity with optimal activity in the neutral pH range.

Preferably, a host cell according to the present invention comprises a polynucleotide encoding AmyB, said polynucleotide comprising a modification, wherein the host cell is deficient in amyBI and/or amyBII compared to the parent cell it originates from when cultivated under comparable conditions. More preferably, a host cell according to the present invention is deficient in amyBI and amy BII. Preferably, the amyB a is a fungal amyB. More preferably, the amyB is the amyB from Aspergillus. Even more preferably the amyB is the amyBI from Aspergillus niger. Even more preferably the amyB is the amyBI from Aspergillus niger CBS 513.88. Most preferably, the amyBI comprises the sequence of An12g06930. Even more preferably the amyB is the amyBII from Aspergillus niger. Even more preferably the amyB is the amyBII from Aspergillus nigerCBS 513.88. Most preferably, the amyBII comprises the sequence of An05g02100.

The term toxin associated polynucleotide is defined herein as a gene cluster, a multitude of genes, a gene or part thereof encoding a compound, or biochemical pathway responsible for the biosynthesis or secretion of at least one toxin or toxin intermediate compound. Said compound may e.g. be a polypeptide, which may be an enzyme.

A number of host cells, especially filamentous fungal host cells, which are used as for the production of polypeptides of interest, comprise genes encoding enzymes involved in the biosynthesis of various toxins. For example, cyclopiazonic acid, kojic acid, 3-nitropropionic acid and aflatoxins are known toxins, which are formed in, e.g., Aspergillus flavus. Similarly, trichothecenes are formed in a number of filamentous fungi, e.g., in Fusarium sp. such as Fusarium venenatum as well as in Trichoderma; ochratoxin may be produced by Aspergillus. Recently, sequencing of the genome of an industrial Aspergillus niger host strain revealed an inactive fumonisin gene cluster (Pel et al., “Genome sequencing and analysis of the versatile cell factory Aspergillus niger CBS 513.88”. Nat Biotechnol. 2007 February; 25 (2):221-231). The formation of such toxins during the fermentation of compounds of interest is highly undesirable as these toxins may present a health hazard to operators, customers and the environment. Consequently, a toxin deficient host cell enables toxin-free production of a compound of interest. The toxin-free compound is easier to produce since no toxin has to be removed from the product. Furthermore, the regulatory approval procedure for the compound is easier.

Preferably, a host cell according to the present invention comprises a toxin associated polynucleotide encoding a compound (which may e.g. be a polypeptide which may be an enzyme) or biochemical pathway, said toxin associated polynucleotide comprising a modification, wherein the host cell is deficient in the production of said toxin or a toxin intermediate compound compared to the parent cell it originates from when cultivated under comparable conditions. Preferably, the toxin or toxin intermediate compound is a fungal toxin or toxin intermediate compound. More preferably, the toxin or toxin intermediate compound is a toxin or toxin intermediate compound from Aspergillus. Even more preferably the toxin or the toxin intermediate compound is a toxin or toxin intermediate compound from Aspergillus niger. Even more preferably the toxin or toxin intermediate compound is a toxin or toxin intermediate compound from Aspergillus niger CBS 513.88. Even more preferably, the toxin or the toxin intermediate compound is fumonisin or a fumonisin intermediate compound. Even more preferably, the toxin or the toxin intermediate compound is ochratoxin or an ochratoxin intermediate compound. Most preferably, the toxin or the toxin intermediate compound is ochratoxin or fumonisin or an ochratoxin or a fumonisin intermediate compound.

Preferably, the toxin associated polynucleotide encodes a compound (which may e.g. be a polypeptide which may be an enzyme) or a biochemical pathway which is involved in the production of a fungal toxin or toxin intermediate compound. More preferably, said toxin or toxin intermediate compound is from Aspergillus. Even more preferably, said toxin or toxin intermediate compound is from Aspergillus niger. Even more preferably, said toxin or toxin intermediate compound is from Aspergillus niger CBS 513.88. Even more preferably, said toxin or toxin intermediate compound is a fumonisin or a fumonisin intermediate compound; even more preferably, a fumonisin-B or a fumonisin-B intermediate compound; even more preferably, a fumonisin-B2 or a fumonisin-B2 intermediate compound. Preferably, the toxin associated polynucleotide comprises the sequence of the fumonisin cluster from An01g06820 until An01g06930; more preferably, the toxin associated polynucleotide comprises the sequence of An01g06930. Alternatively or in combination when the toxin or toxin intermediate compound is a fumonisin or a fumonisin intermediate compound, the toxin associated polynucleotide encodes a compound (which may e.g. be a polypeptide which may be an enzyme) or a biochemical pathway, which is involved in ochratoxin or an ochratoxin intermediate compound; preferably, an ochratoxin A or an ochratoxin A intermediate compound; more preferably, the toxin associated polynucleotide comprises the sequence of the cluster from An15g07880 until An15g07930; most preferably, the toxin associated polynucleotide comprises the sequence of An15g07910 and/or the sequence of An15g07920.

Preferably, a host cell according to the present invention comprises at least one toxin associated polynucleotide encoding a compound (which may e.g. be a polypeptide which may be an enzyme) or biochemical pathway, said toxin associated polynucleotide comprising at least one modification, wherein the host cell is deficient in the production of a toxin or, toxin intermediate compound compared to the parent cell it originates from when cultivated under comparable conditions. More preferably, a host cell according to the present invention comprises two toxin associated polynucleotides, said two toxin associated polynucleotides each comprising at least one modification, wherein the host cell is preferably deficient in the production of fumonisin and ochratoxin compared to the parent cell it originates from when cultivated under comparable conditions. Even more preferably, a mutant microbial host cell according to the invention comprises three or more toxin associated polynucleotides, said three or more toxin associated polynucleotides each comprising at least one modification, wherein the host cell is preferably deficient in the production of fumonisin, ochratoxin and at least one additional toxin or toxin intermediate compound compared to the parent cell it originates from when cultivated under comparable conditions.

Preferably, a host cell according to the present invention comprises one or more modifications in its genome to result in a deficiency in the production of the major extracellular aspartic protease PepA. Preferably, the host cell according to the present invention comprises a disruption of the pepA gene encoding the major extracellular aspartic protease PepA; more preferably, the pepA is the pepA from Aspergillus; even more preferably the pepA is the pepA from Aspergillus niger even more preferably the pepA is the pepA from Aspergillus niger CBS 513.88; most preferably, the pepA comprises the sequence of An14g04710.

Preferably, the efficiency of targeted integration of a polynucleotide to a pre-determined site into the genome of a host cell according to the invention is increased by rendering the cell deficient in a component in NHEJ (non-homologous recombination). Preferably, a host cell according to the invention comprises a polynucleotide encoding an NHEJ component comprising a modification, wherein said host cell is deficient in the production of said NHEJ component compared to a parent cell it originates from when cultivated under the same conditions.

The NHEJ component to be modified can be any NHEJ component known to the person skilled in the art. Preferred NHEJ components to be modified are selected from the group of filamentous fungal homologues of yeast KU70, KU80, MRE11, RAD50, RAD51, RAD52, XRS2, SIR4, LIG4. More preferred NHEJ components to be modified are filamentous fungal homologues of yeast KU70 and KU80, preferably hdfA (homologue of yeast KU70) or homologues thereof and hdfB (homologue of yeast KU80) or homologues thereof. The most preferred NHEJ component to be modified is KU70 or hdfA, or a homologue thereof. Another preferred NHEJ component to be modified is KU80 or hdfB, or a homologue thereof. Yet another preferred NHEJ component to be modified is a filamentous fungal homologue of yeast LIG4, or a homologue thereof. Methods to obtain such host cell deficient in a component involved in NHEJ are known to the skilled person and are extensively described in WO2005/095624. Preferably, the hdfA gene is the hdfA gene from A. niger, more preferably the hdfA from A. niger according to SEQ ID NO: 1 of WO2005/095624. In another preferred embodiment the hdfB gene is the hdfB gene from A. niger, more preferably the hdfB from A. niger according to SEQ ID NO: 4 of WO2005/095624.

When a host cell according to the present invention is a filamentous fungal host cell, said host cell preferably additionally comprises one or more modifications in its genome to result in a deficiency in the production of the product encoded by the hdf A gene (as depicted in SEQ ID NO: 3 of WO 2005/095624) and/or hdfB gene (as depicted in SEQ ID NO: 6 of WO 2005/095624). A host cell according to the present invention preferably further comprises a disruption of the hdfA and/or hdfB gene. Filamentous fungal host cells which are deficient in a product encoded by the hdfA and/or hdfB gene have been described in WO 2005/095624.

When a host cell according to the present invention is a filamentous fungal host cell, said host cell preferably further comprises a modification in its genome which results in the deficiency in the production of the non-ribosomal peptide synthase npsE, preferably the npsE depicted in SEQ ID NO: 38 of WO2012/001169. Such host cells deficient in the production of non-ribosomal peptide synthase npsE have been described in WO2012/001169 (npsE has a genomic sequence as depicted in SEQ ID NO: 35, a coding sequence as depicted in SEQ ID NO: 36, an mRNA as depicted in SEQ ID NO: 37 and the nrps protein as depicted in SEQ ID NO: 38 of WO2012/001169).

A host cell according to the present invention preferably further comprises a modification in its genome which results in the deficiency in the production of the α-amylase amyC, preferably the mature AmyC protein shown in SEQ ID NO: 4 and 8 of WO2014/013073. Such host cells deficient in the production of the α-amylase amyC have been described in WO2014/013073. amyC has a genomic sequence as depicted in SEQ ID NO: 1 or 5 and a coding sequence depicted in SEQ ID NO: 2 or 6 and the AmyC protein as depicted in SEQ ID NO: 3 or 7 with the mature AmyC protein shown in SEQ ID NO: 4 and 8 of WO2014/013073).

A host cell according to the present invention preferably further comprises a modification in its genome which results in the deficiency in the production of the AgsE protein, preferably the mature AgsE protein shown in SEQ ID NO: 3 or comprised in SEQ ID NO: 3 of WO2014/013074. Such host cells deficient in the production of the AgsE protein have been described in WO2014/013074. AgsE has a genomic sequence as depicted in SEQ ID NO: 1 and a coding sequence depicted in SEQ ID NO: 2 and the AgsE protein as depicted in SEQ ID NO: 3 with the mature AgsE protein comprised in SEQ ID NO: 3 of WO2014/013074).

The deficiency in the production of at least one product selected from glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, amylase amyC if compared to a parent host cell and measured under the same conditions may already be present in a parent host cell from which a host cell according to the present invention that is deficient in a further product selected from the group consisting of glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, amylase amyC is derived. The deficiency in the production of at least one product selected from glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, amylase amyC, protein AgsE if compared to a parent host cell and measured under the same conditions may already be present in a parent host cell from which a host cell according to the present invention that is deficient in a further product selected from the group consisting of glucoamylase (glaA), acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, amylase amyC, protein AgsE is derived. A preferred host cell according to the present invention comprises a deficiency in the production of glaA and optionally at least another product selected from the group consisting of acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, amylase amyC if compared to a parent host cell and measured under the same conditions.

A further preferred host cell according to the present invention comprises a deficiency in the production of glaA, PepA and optionally at least another product selected from the group consisting of acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions. A further preferred host cell according to the present invention comprises a deficiency in the production of glaA, PepA, acid stable alpha-amylase (amyA) and optionally at least another product selected from the group consisting of neutral alpha-amylase (amyBI and amyBII), oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions. A further preferred host cell according to the present invention comprises a deficiency in the production of glaA, PepA, acid stable alpha-amylase (amyA), neutral alpha-amylase amyBI and optionally at least another product selected from the group consisting of neutral alpha-amylase amyBII, oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions.

A further preferred host cell according to the present invention comprises a deficiency in the production of glaA, PepA, acid stable alpha-amylase (amyA), neutral alpha-amylase amyBI and amyBII, and optionally at least another product selected from the group consisting of oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC if compared to a parent host cell and measured under the same conditions. A further preferred host cell according to the present invention comprises a deficiency in the production of glaA, PepA, acid stable alpha-amylase (amyA), neutral alpha-amylase amyBI and amyBII, a product encoded by the gene hdfA and optionally at least another product selected from the group consisting of oxalic acid hydrolase (oahA), a toxin, preferably ochratoxin and/or fumonisin, a protease transcriptional regulator prtT, a product encoded by the gene hdfB, a non-ribosomal peptide synthase npsE, amylase amyC if compared to a parent host cell and measured under the same conditions.

A further preferred host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA) and optionally at least another product selected from the group consisting of, a toxin, preferably ochratoxin and/or fumonisin, a protease transcriptional regulator prtT, a product encoded by the gene hdfB, a non-ribosomal peptide synthase npsE, amylase amyC if compared to a parent host cell and measured under the same conditions.

A further preferred host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), ochratoxin, fumonisin, and optionally at least another product selected from the group consisting of a protease transcriptional regulator prtT, a product encoded by the gene hdfB, a non-ribosomal peptide synthase npsE, amylase amyC if compared to a parent host cell and measured under the same conditions.

A further preferred host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), ochratoxin, fumonisin, a protease transcriptional regulator prtT and optionally at least another product selected from the group consisting of a product encoded by the gene hdfB, a non-ribosomal peptide synthase npsE, amylase amyC if compared to a parent host cell and measured under the same conditions.

A further preferred host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), ochratoxin, fumonisin, a protease transcriptional regulator prtT, a non-ribosomal peptide synthase npsE and optionally at least another product selected from the group consisting of a product encoded by the gene hdfB, amylase amyC if compared to a parent host cell and measured under the same conditions.

A further preferred host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), ochratoxin, fumonisin, a protease transcriptional regulator prtT, amylase amyC and optionally at least another product selected from the group consisting of a product encoded by the gene hdfB, a non-ribosomal peptide synthase npsE, if compared to a parent host cell and measured under the same conditions.

A further preferred host cell according to the present invention comprises a reduced amylase background and comprises a deficiency in the production of glaA, acid stable alpha-amylase (amyA), neutral alpha-amylase amyBI and amyBII, if compared to a parent host cell and measured under the same conditions. Such host cell preferably also comprises a deficiency in the production of a filamentous fungal homolog of KU70 or KU80. Such host cell preferably also comprises a deficiency in the production of a toxin. Such a host cell preferably also comprises a deficiency in the production of a filamentous fungal homolog of KU70 or KU80 and a deficiency in the production of a toxin.

A further preferred host cell according to the present invention comprises a reduced amylase background and further comprises a deficiency in the production of glaA, acid stable alpha-amylase (amyA), neutral alpha-amylase amyBI, amyBII and amyC if compared to a parent host cell and measured under the same conditions. Such a host cell may preferably also comprises a filamentous fungal homolog of KU70 or KU80. Such host cell preferably also comprises a deficiency in the production of a toxin. Such host cell preferably also comprises a deficiency in the production of a filamentous fungal homolog of KU70 or KU80 and a deficiency in the production of a toxin.

A preferred host cell according to the present invention is a filamentous fungal host cell which comprises a deficiency in the production of glaA and optionally at least another product selected from the group consisting of acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, PepA, a product encoded by the gene hdfA and/or hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, and optionally at least another product selected from the group consisting of acid stable alpha-amylase (amyA), neutral alpha-amylase (amyBI and amyBII), oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA) and optionally at least another product selected from the group consisting of neutral alpha-amylase (amyBI and amyBII), oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and optionally at least another product selected from the group consisting of neutral alpha-amylase amyBII, oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII and optionally at least another product selected from the group consisting of oxalic acid hydrolase (oahA), a toxin, preferably ochratoxin and/or fumonisin, a protease transcriptional regulator prtT, a product encoded by the gene hdfA and/or hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA and optionally at least another product selected from the group consisting of oxalic acid hydrolase (oahA), a toxin, preferably ochratoxin and/or fumonisin, a protease transcriptional regulator prtT, a product encoded by the gene hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), and optionally at least another product selected from the group consisting of a toxin, preferably ochratoxin and/or fumonisin, a protease transcriptional regulator prtT, a product encoded by the gene hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), a protein AgsE and optionally at least another product selected from the group consisting of a toxin, preferably ochratoxin and/or fumonisin, a protease transcriptional regulator prtT, a product encoded by the gene hdfB, a non-ribosomal peptide synthase npsE, amylase amyC, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), a protein AgsE, a toxin, preferably ochratoxin and/or fumonisin, and optionally at least another product selected from the group consisting of a protease transcriptional regulator prtT, a product encoded by the gene hdfB, a non-ribosomal peptide synthase npsE, amylase amyC, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, oxalic acid hydrolase (oahA), a protein AgsE, a toxin, preferably ochratoxin and/or fumonisin, amylase amyC, and optionally at least another product selected from the group consisting of a protease transcriptional regulator prtT, a product encoded by the gene hdfB, a non-ribosomal peptide synthase npsE, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, and optionally at least another product selected from the group consisting of oxalic acid hydrolase (oahA), a protease transcriptional regulator prtT, a product encoded by the gene hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, amylase amyC, and optionally at least another product selected from the group consisting of oxalic acid hydrolase (oahA), a protease transcriptional regulator prtT, a product encoded by the gene hdfB, a non-ribosomal peptide synthase npsE, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, a non-ribosomal peptide synthase npsE, and optionally at least another product selected from the group consisting of oxalic acid hydrolase (oahA), a protease transcriptional regulator prtT, a product encoded by the gene hdfB, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, a protein AgsE, and optionally at least another product selected from the group consisting of oxalic acid hydrolase (oahA), a protease transcriptional regulator prtT, a product encoded by the gene hdfB, a non-ribosomal peptide synthase npsE, amylase amyC, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, a protein AgsE, amylase amyC, and optionally at least another product selected from the group consisting of oxalic acid hydrolase (oahA), a protease transcriptional regulator prtT, a product encoded by the gene hdfB, a non-ribosomal peptide synthase npsE, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, a protein AgsE, a non-ribosomal peptide synthase npsE, and optionally at least another product selected from the group consisting of oxalic acid hydrolase (oahA), a protease transcriptional regulator prtT, a product encoded by the gene hdfB, amylase amyC, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, amylase amyC, a non-ribosomal peptide synthase npsE, and optionally at least another product selected from the group consisting of oxalic acid hydrolase (oahA), a protease transcriptional regulator prtT, a protein AgsE, a product encoded by the gene hdfB, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, a protein AgsE, amylase amyC, a non-ribosomal peptide synthase npsE, and optionally at least another product selected from the group consisting of oxalic acid hydrolase (oahA), a protease transcriptional regulator prtT, a product encoded by the gene hdfB, if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, oxalic acid hydrolase (oahA), and optionally at least another product selected from the group consisting of a protease transcriptional regulator prtT, a product encoded by the gene hdfB, a non-ribosomal peptide synthase npsE, amylase amyC, a protein AgsE if compared to a parent host cell and measured under the same conditions.

In one embodiment the host cell according to the present invention comprises a deficiency in the production of glaA, PepA, acid stable alpha-amylase (amyA), neutral alpha-amylase amyBI and amyBII, a product encoded by the gene hdfA, a toxin, preferably ochratoxin and/or fumonisin, oxalic acid hydrolase (oahA), a non-ribosomal peptide synthase npsE, and optionally at least another product selected from the group consisting of a protease transcriptional regulator prtT, a product encoded by the gene hdfB, amylase amyC, a protein AgsE if compared to a parent host cell and measured under the same conditions.

A further preferred host cell according to the present invention comprises a reduced alpha-amylase background and comprises a deficiency in the production of acid stable alpha-amylase (amyA), neutral alpha-amylase amyBI and amyBII and, optionally, amyC if compared to a parent host cell and measured under the same conditions. Such host cell preferably also comprises a filamentous fungal homolog of KU70 or KU80. Such host cell preferably also comprise a deficiency in the production of a toxin. Such host cell preferably also comprises a deficiency in the production of a filamentous fungal homolog of KU70 or KU80 and a deficiency in the production of a toxin.

When a host cell according to the present invention is a filamentous fungal host cell, the host cell preferably further comprises at least two substantially homologous DNA domains suitable for integration of one or more copies of a polynucleotide according to the present invention or of a polynucleotide encoding a compound of interest, wherein at least one of the at least two substantially homologous DNA domains is adapted to have enhanced integration preference for the polynucleotide encoding a compound of interest compared to the substantially homologous DNA domain it originates from, and wherein the substantially homologous DNA domain where the adapted substantially homologous DNA domain originates from has a gene conversion frequency that is at least 10% higher than one of the other of the at least two substantially homologous DNA domains. Such host cells have extensively been described in WO2011/009700. Strains containing two or more copies of these substantially homologous DNA domains are also referred herein as strain containing two or more amplicons. Examples of host cells comprising such amplicons are inter alia described in van Dijck et al, 2003, Regulatory Toxicology and Pharmacology 28; 27-35: On the safety of a new generation of DSM Aspergillus niger enzyme production strains. In van Dijck et al, an Aspergillus niger strain is described that comprises 7 amplified glucoamylase gene loci, i.e. 7 amplicons. Preferred host cells according to the present invention are filamentous fungus host cells, preferably A. niger host cells, comprising two or more amplicons, preferably two or more ΔglaA amplicons, more preferably comprising 2, 3, 4, 5, 6, 7 ΔglaA amplicons, wherein the amplicon which has the highest frequency of gene conversion has been adapted to have enhanced integration preference for the polynucleotide according to the present invention or the polynucleotide encoding a compound of interest, compared to the amplicon it originates from. Adaptation of the amplicon can be performed according to any one of the methods described in WO2011/009700 (which is here fully incorporated by reference). Host cells comprising two or more amplicons wherein one amplicon has been adapted to have enhanced integration preference for a polynucleotide encoding a compound of interest compared to the amplicon it originates from are herein referred as host cells comprising an adapted amplicon. Preferred host cells with adapted amplicons, described in WO2011/009700, are host cells comprising three ΔglaA amplicons being a BamHI truncated amplicon, a SalI truncated amplicon and a BglII truncated amplicon and wherein the BamHI amplicon has been adapted to have enhanced integration preference for a polynucleotide according to the present invention or polynucleotide encoding a compound of interest, compared to the BamHI amplicon it originates from.

When a host cell according to the present invention is a filamentous fungal host cell, the host cell according to the present invention preferably further comprises a modification of Sec61. A preferred SEC61 modification is a modification which results in a one-way mutant of SEC61; i.e. a mutant wherein the de novo synthesized protein can enter the ER via SEC61, but the protein cannot leave the ER via SEC61. Such modifications are extensively described in WO2005/123763. In a preferred embodiment the mutant microbial host cell comprises a modification in a Sec61 as depicted in SEQ ID NO: 3 of WO2005/123763. Most preferably, the SEC 61 modification is the S376W mutation in which Serine 376 is replaced by Tryptophan in SEQ ID NO: 3 of WO2005/123763.

A modification, preferably in the genome, is construed herein as one or more modifications. A modification, preferably in the genome of a host cell according to the present invention, can either be effected by
a) subjecting a parent host cell to recombinant genetic manipulation techniques; and/or
b) subjecting a parent host cell to (classical) mutagenesis; and/or
c) subjecting a parent host cell to an inhibiting compound or composition. Modification of a genome of a host cell is herein defined as any event resulting in a change in a polynucleotide sequence in the genome of the host cell.

Preferably, a host cell according to the present invention has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes for a partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In one embodiment the mutant microbial host cell according to the invention comprises a modification in its genome selected from
a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32, (7) electronic access http://nar.oupjournals.org/cgi/reprint/32/7/e59 or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene replacement or partial or complete gene deletion.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example a polynucleotide corresponding to the endogenous polynucleotide may be replaced by a defective polynucleotide, that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified. Alternatively or in combination with other mentioned techniques, a technique based on in vivo recombination of cosmids in E. coli can be used, as described in: A rapid method for efficient gene replacement in the filamentous fungus Aspergillus nidulans (2000) Chaveroche, M-K., Ghico, J-M. and d'Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.).

A modification resulting in reduced or no production of undesired compound is preferably due to a reduced production of the mRNA encoding said undesired compound if compared with a parent microbial host cell which has not been modified and when measured under the same conditions. A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in WO2008/053019, WO2005/05672A1, WO2005/026356A1, Oliveira et al., Crook et al., 2014; and/or Barnes et al., may be used at this purpose.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath_et al, (2003) Nature: Systematic functional analysis of the Caenorhabditis elegans genome using RNAi.vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266(4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6.) or by the use of inhibitors inhibiting enzymatic activity of an undesired polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)).

In an embodiment of the present invention, the modification in the genome of the host cell according to the invention is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell according to the present invention produces 1% less of the un-desired compound if compared with the parent host cell which has not been modified and measured under the same conditions, at least 5% less of the un-desired compound, at least 10% less of the un-desired compound, at least 20% less of the un-desired compound, at least 30% less of the un-desired compound, at least 40% less of the un-desired compound, at least 50% less of the un-desired compound, at least 60% less of the un-desired compound, at least 70% less of the un-desired compound, at least 80% less of the un-desired compound, at least 90% less of the un-desired compound, at least 91% less of the un-desired compound, at least 92% less of the un-desired compound, at least 93% less of the un-desired compound, at least 94% less of the un-desired compound, at least 95% less of the un-desired compound, at least 96% less of the un-desired compound, at least 97% less of the un-desired compound, at least 98% less of the un-desired compound, at least 99% less of the un-desired compound, at least 99.9% less of the un-desired compound, or most preferably 100% less of the un-desired compound.

A reference herein to a patent document or other matter which is given as prior art is not to be taken as an admission that that document or matter was known or that the information it contains was part of the common general knowledge as at the priority date of any of the claims.

The sequence information as provided herein should not be so narrowly construed as to require inclusion of erroneously identified bases. The skilled person is capable of identifying such erroneously identified bases and knows how to correct for such errors.

The disclosure of each reference set forth herein is incorporated herein by reference in its entirety.

The invention is illustrated by the following, non limiting embodiments:

### EMBODIMENTS OF THE INVENTION

1. Method to produce a vector, preferably a circular vector comprising one or more guide-polynucleotide expression cassettes, wherein said one or more guide-polynucleotide expression cassettes comprise a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following steps:
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes;
subjecting the one or more hybrid linear polynucleotides obtained in the overlap-extension PCR and optionally one or more additional linear polynucleotides to an assembly reaction yielding a vector, preferably a circular vector comprising one or more functional guide-polynucleotide expression cassettes,

wherein the assembly reaction occurs in vivo.

2. The method of embodiment 1 wherein the guide-polynucleotide is a gRNA.

3. The method according to any one of the preceding embodiments wherein the fragment of a guide-polynucleotide expression cassette at the 5′-end and/or at the 3′-end of the polynucleotide sequence to be extended, does not comprise a polynucleotide coding for a guide sequence.

4. The method according to any one of the preceding embodiments wherein the vector, preferably the circular vector comprises two or more guide-polynucleotide expression cassettes, preferably wherein the method comprises the following steps:
providing two or more polynucleotides, wherein the two or more polynucleotides comprise a polynucleotide sequence to be extended, wherein the polynucleotide sequence to be extended comprises at the 5′-end and/or at the 3′-end to be extended a fragment of a guide-polynucleotide expression cassette;
performing two or more overlap-extension PCR reactions by subjecting in each reaction one of said two or more polynucleotides and two suitable polynucleotide primers to yield one hybrid linear polynucleotide,

wherein the two or more polynucleotide sequence to be extended and the suitable polynucleotide primers are selected so that each hybrid linear polynucleotide obtained in the two or more overlap-extension PCR reactions comprises at least a polynucleotide coding for a guide sequence and suitable 5′-termini and 3′-termini which allow assembly in vivo of the two or more hybrid linear polynucleotides and optionally of one or more additional linear polynucleotides with each other in a pre-defined order to yield a vector, preferably a circular vector comprising one or more functional guide-polynucleotide expression cassettes;
subjecting the two or more hybrid linear polynucleotides obtained in the overlap-extension PCR and optionally one or more additional linear polynucleotides to an assembly reaction yielding a vector, preferably a circular vector comprising two or more functional guide-polynucleotide expression cassettes,

wherein the assembly reaction occurs in vivo.

5. The method according to any one of embodiments 1 to 3 wherein the vector, preferably the circular vector comprises one guide-polynucleotide expression cassette, preferably wherein the method comprises the following steps:
providing one polynucleotide, wherein said polynucleotide comprises a polynucleotide sequence to be extended, wherein the polynucleotide sequence to be extended comprises at the 5′-end and/or at the 3′-end to be extended a fragment of a guide-polynucleotide expression cassette;
performing one overlap-extension PCR reactions by subjecting in the reaction said polynucleotide and two suitable polynucleotide primers to yield one hybrid linear polynucleotide,

wherein the polynucleotide sequence to be extended and the suitable polynucleotide primers are selected so that the hybrid linear polynucleotide obtained in the overlap-extension PCR reaction comprises at least a polynucleotide coding for a guide sequence and suitable 5′-termini and 3′-termini which allow assembly in vivo of the hybrid linear polynucleotides and optionally of one or more additional linear polynucleotides with each other in a pre-defined order to yield a vector, preferably a circular vector comprising one or more functional guide-polynucleotide expression cassettes;
subjecting the hybrid linear polynucleotide obtained in the overlap-extension PCR and optionally one or more additional linear polynucleotides to an assembly reaction yielding a vector, preferably a circular vector comprising one or more functional guide-polynucleotide expression cassettes,

wherein the assembly reaction occurs in vivo.

6. The method according to any one of embodiments 1 to 5, wherein said performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes,

comprises the steps of:

a) subjecting in each reaction one of said one or more polynucleotides and one suitable polynucleotide primer to overlap-extension PCR reaction, wherein the 3′-terminus of said polynucleotide primer contains a complementary sequence to the 3′-end of the polynucleotide sequence to be extended comprised in said one or more polynucleotide or wherein the 3′-terminus of the complement of said polynucleotide primer contains a complementary sequence to the 3′-end of the complement of the polynucleotide sequence to be extended comprised in said one or more polynucleotide, respectively, to yield one hybrid linear polynucleotide having a sequence comprising the polynucleotide sequence to be extended spliced at its 3′-terminus or at its 5′-terminus, respectively, with the polynucleotide sequence of the suitable primer,

b) optionally subjecting the hybrid linear polynucleotide obtained in step a) and another suitable primer to a second overlap-extension PCR reaction, wherein the 3′-terminus of said polynucleotide primer contains a complementary sequence to the 3′-end of the hybrid linear polynucleotide having a sequence comprising the polynucleotide sequence to be extended spliced at its 3′-terminus or at its 5′-terminus, respectively, with the polynucleotide sequence of the suitable primer, or wherein the 3′-terminus of the complement of said polynucleotide primer contains a complementary sequence to the 3′-end of the complement of the hybrid linear polynucleotide having a sequence comprising the polynucleotide sequence to be extended spliced at its 3′-terminus or at its 5′-terminus, respectively, with the polynucleotide sequence of the other suitable primer, respectively, to yield one hybrid linear polynucleotide having a sequence comprising the polynucleotide sequence to be extended spliced at its 3′-terminus and at its 5′-terminus, respectively, with the polynucleotide sequences of the suitable primers,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

7. The method according to any one of embodiments 1 to 6 wherein the one or more polynucleotide sequence to be extended or additional linear polynucleotide comprises one or more elements selected from the group consisting of: an origin of replication or a fragment thereof, a selectable marker or a fragment thereof, a CAS9 expression cassette or a fragment thereof, a donor polynucleotide or a fragment thereof.

8. The method according to embodiment 7 wherein the vector, preferably the circular vector obtained after assembly comprises one or more elements selected from the group consisting of: an origin of replication, a selectable marker, a CAS9 expression cassette, a donor polynucleotide or a combination of one or more of said elements.

9. The method according to embodiment 8 wherein the vector, preferably the circular vector obtained after assembly further comprises an origin of replication, preferably an origin of replication and a selectable marker.

10. The method according to any one of the preceding embodiment wherein the primers used in each overlap-extension PCR reaction are at most 100 nucleotides long, preferably at most 80, 70, 60, 50, 40, 30 nucleotides long.

11. The method according to any one of the preceding embodiments wherein the primers used in each overlap-extension PCR reaction are at least 30 nucleotides long, preferably at least 40, 50, 60 nucleotides long.

12. A method of assembling a vector, preferably a circular vector comprising one or more guide-polynucleotide expression cassettes in vivo, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following steps:
providing one or more linear polynucleotides, wherein said one or more linear polynucleotides comprise at the 5′-terminus and/or at the 3′-terminus at least one guide-polynucleotide expression cassette or a fragment of at least one guide-polynucleotide expression cassette comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes;
optionally selecting a host cell comprising a vector, preferably a circular vector and wherein said vector comprises one or more functional guide-polynucleotide expression cassettes;
optionally recovering the vector, preferably the circular vector.

13. A method for producing a recombinant host cell comprising a vector, preferably a circular vector, wherein said vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following steps:
providing one or more linear polynucleotides, wherein said one or more linear polynucleotides comprise at the 5′-terminus and/or at the 3′-terminus at least one guide-polynucleotide expression cassette or a fragment of at least one guide-polynucleotide expression cassette comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a vector, preferably a circular vector comprising one or more functional guide-polynucleotide expression cassettes;
optionally selecting a host cell comprising a vector, preferably a circular vector and wherein said vector comprises one or more functional guide-polynucleotide expression cassettes.

14. A method for producing a recombinant host cell according to embodiment 13 further comprising the step of:
recovering the vector, preferably the circular vector;
transforming the vector in a second host cell;
optionally isolating the second host cell comprising the vector.

15. A method for producing a recombinant host cell comprising a vector, preferably a circular vector, wherein said vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following steps:
performing a method of assembling a vector, preferably a circular vector comprising one or more guide-polynucleotide expression cassettes in vivo according to embodiment 10 in a first host cell, preferably a first host cell belonging to the species S. cerevisiae, wherein said first host cell comprising the vector or circular vector, is selected and the vector or circular vector is recovered, wherein said vector or circular vector comprises one or more functional guide-polynucleotide expression cassettes,
transforming the vector, preferably the circular vector in a second host cell;
optionally isolating the second host cell comprising the vector or circular vector.

16. A method according to any one of embodiments 12 to 15 wherein the one or more linear polynucleotides are hybrid linear polynucleotides obtained by
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette, preferably wherein the fragment of a guide-polynucleotide expression cassette at the 5′-end and/or at the 3′-end of the polynucleotide sequence to be extended, does not comprise a polynucleotide coding for a guide sequence;
performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

17. A method according to embodiment 16 wherein the one or more linear polynucleotides are two or more hybrid linear polynucleotides preferably obtained by:
providing two or more polynucleotides, wherein the two or more polynucleotides comprise a polynucleotide sequence to be extended, wherein the polynucleotide sequence to be extended comprises at the 5′-end and/or at the 3′-end to be extended a fragment of a guide-polynucleotide expression cassette, preferably wherein the fragment of a guide-polynucleotide expression cassette at the 5′-end and/or at the 3′-end of the polynucleotide sequence to be extended, does not comprise a polynucleotide coding for a guide sequence;
performing two or more overlap-extension PCR reactions by subjecting in each reaction one of said two or more polynucleotides and two suitable polynucleotide primers to yield one hybrid linear polynucleotide,

wherein the two or more polynucleotide sequence to be extended and the suitable polynucleotide primers are selected so that each hybrid linear polynucleotide obtained in the two or more overlap-extension PCR reactions comprises at least a polynucleotide coding for a guide sequence and suitable 5′-termini and 3′-termini which allow assembly in vivo of the two or more hybrid linear polynucleotides and optionally of one or more additional linear polynucleotides with each other in a pre-defined order to yield a vector, preferably a circular vector comprising one or more functional guide-polynucleotide expression cassettes.

18. The method according to embodiment 16 wherein the one or more linear polynucleotides is one hybrid linear polynucleotides preferably obtained by:
providing one polynucleotide, wherein said polynucleotide comprises a polynucleotide sequence to be extended, wherein the polynucleotide sequence to be extended comprises at the 5′-end and/or at the 3′-end to be extended a fragment of a guide-polynucleotide expression cassette, preferably wherein the fragment of a guide-polynucleotide expression cassette at the 5′-end and/or at the 3′-end of the polynucleotide sequence to be extended, does not comprise a polynucleotide coding for a guide sequence;
performing one overlap-extension PCR reactions by subjecting in the reaction said polynucleotide and two suitable polynucleotide primers to yield one hybrid linear polynucleotide,

wherein the polynucleotide sequence to be extended and the suitable polynucleotide primers are selected so that the hybrid linear polynucleotide obtained in the overlap-extension PCR reaction comprises at least a polynucleotide coding for a guide sequence and suitable 5′-termini and 3′-termini which allow assembly in vivo of the hybrid linear polynucleotides and optionally of one or more additional linear polynucleotides with each other in a pre-defined order to yield a vector, preferably a circular vector comprising one or more functional guide-polynucleotide expression cassettes.

19. A method according to any one of embodiments 16 to 18 wherein said performing one or more overlap-extension PCR reactions by subjecting in each reaction one of said one or more polynucleotides and two suitable polynucleotide primers to yield one hybrid linear polynucleotide,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other to yield a vector, preferably a circular vector comprising one or more functional guide-polynucleotide expression cassettes, comprises the steps of:

a) subjecting in each reaction one of said one or more polynucleotides and one suitable polynucleotide primer to overlap-extension PCR reaction, wherein the 3′-terminus of said polynucleotide primer contains a complementary sequence to the 3′-end of the polynucleotide sequence to be extended comprised in said one or more polynucleotide or wherein the 3′-terminus of the complement of said polynucleotide primer contains a complementary sequence to the 3′-end of the complement of the polynucleotide sequence to be extended comprised in said one or more polynucleotide, respectively, to yield one hybrid linear polynucleotide having a sequence comprising the polynucleotide sequence to be extended spliced at its 3′-terminus or at its 5′-terminus, respectively, with the polynucleotide sequence of the suitable primer,

b) optionally subjecting the hybrid linear polynucleotide obtained in step a) and another suitable primer to a second overlap-extension PCR reaction, wherein the 3′-terminus of said polynucleotide primer contains a complementary sequence to the 3′-end of the hybrid linear polynucleotide having a sequence comprising the polynucleotide sequence to be extended spliced at its 3′-terminus or at its 5′-terminus, respectively, with the polynucleotide sequence of the suitable primer, or wherein the 3′-terminus of the complement of said polynucleotide primer contains a complementary sequence to the 3′-end of the complement of the hybrid linear polynucleotide having a sequence comprising the polynucleotide sequence to be extended spliced at its 3′-terminus or at its 5′-terminus, respectively, with the polynucleotide sequence of the other suitable primer, respectively, to yield one hybrid linear polynucleotide having a sequence comprising the polynucleotide sequence to be extended spliced at its 3′-terminus and at its 5′-terminus, respectively, with the polynucleotide sequences of the suitable primers,

wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a vector, preferably a circular vector comprising one or more functional guide-polynucleotide expression cassettes.

20. A method according to any one of the preceding embodiments, wherein the assembly reaction occurs in a host cell belonging to S. cerevisiae species.

21. A method according to any one of embodiments 15 to 20 wherein the second host cell is a prokaryotic, such as a bacterial host cell, or an eukaryotic host cell, preferably a fungal host cell.

22. A method according to embodiment 21 wherein the second host cell is a prokaryotic host cell selected from Escherichia, Anabaena, Caulobactert, Gluconobacter, Rhodobacter, Pseudomonas, Paracoccus, Bacillus, Brevibacterium, Corynebacterium, Rhizobium (Sinorhizobium), Flavobacterium, Klebsiella, Enterobacter, Lactobacillus, Lactococcus, Methylobacterium, Staphylococcus or Streptomyces.

23. A method according to embodiment 21 wherein the second host cell is a fungal host cell, more preferably a filamentous fungal host cell, most preferably a filamentous fungal host cell selected from Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma.

24. A method according to embodiment 23, wherein the fungal host cell is a yeast host cell, preferably a yeast host cell selected from Candida, Hansenula, Issatchenkia, Kluyveromyces, Pichia, Saccharomyces, Schizosaccharomyces, Yarrowia or Zygosaccharomyces.

25. The method according to anyone of embodiments 1 to 24 wherein the fragment of a guide-polynucleotide expression cassette present at the 3′-end of the one or more polynucleotide sequence to be extended is the same in said one or more polynucleotides.

26. The method of any one of embodiments 1 to 25 wherein the fragment the guide-polynucleotide expression cassette present at the 5′-end of the one or more polynucleotidesequence to be extended is the same in said one or more polynucleotides.

27. The method according to anyone of embodiments 1 to 26 wherein the fragment of a guide polynucleotide expression cassette present at the 3′-end of the one or more polynucleotide sequence to be extended comprises or consists of a promoter polynucleotide sequence.

28. The method of anyone of embodiments 1 to 27 wherein the fragment of a guide polynucleotide expression cassette present at the 5′-end of the one or more polynucleotide sequence to be extended comprises or consists of a terminator polynucleotide sequence.

29. The method according to anyone of embodiments 1 to 28 wherein the vector, preferably the circular vector comprises at least 2 or more functional guide polynucleotide expression cassettes

30. A vector, preferably a circular vector comprising one or more functional guide polynucleotide expression cassettes obtainable by the method of any one of embodiments 1 to 12 or 16 to 29.

31. A recombinant host cell comprising a vector, preferably a circular vector said vector or circular vector comprising one or more functional guide polynucleotides expression cassettes which recombinant host cell is obtainable by the method of any one of embodiments 13 to 29.

32. Use of a vector, preferably a circular vector according to claim 30 or of a host cell comprising a circular vector according to embodiment 27 in a CRISP-CAS mediated transformation.

33. A non-naturally occurring or engineered composition comprising a source of a CRISPR-Cas system comprising a guide-polynucleotide and a Cas protein, wherein the guide-polynucleotide comprises a guide-sequence that essentially is the reverse complement of a target-polynucleotide in a host cell and wherein the guide-polynucleotide can direct binding of the Cas protein at the target-polynucleotide in the host cell to form a CRISPR-Cas complex, wherein the guide-sequence is essentially the reverse complement of the (N)y part of a 5′-(N)yPAM-3′ polynucleotide sequence target in the genome of the host cell, wherein y is an integer of 8-30, wherein PAM is a protospacer adjacent motif, wherein the host cell is preferably a prokaryote or a eukaryote, and wherein PAM is preferably a sequence selected from the group consisting of 5′-XGG-3′, 5′-XGGXG-3′, 5′-XXAGAAW-3′, 5′-XXXXGATT-3′, 5′-XXAGAA-3′, 5′-XAAAAC-3′, wherein X can be any nucleotide or analog thereof, preferably X can be any nucleotide; and W is A or T, wherein the guide-polynucleotide in said composition and optionally a Cas protein in said composition is comprised in a vector, preferably a circular vector according to embodiment 30.

34. Method of modulating expression of a polynucleotide in a host cell, comprising contacting a host cell with the composition according to embodiment 33, wherein the guide-polynucleotide directs binding of the Cas protein at the target-polynucleotide in the host cell to form a CRISPR-Cas complex, preferably wherein the host cell comprises a polynucleotide encoding a compound of interest.

35. Method for the production of a compound of interest, comprising culturing under conditions conducive to the production of the compound of interest a recombinant host cell according to embodiment 31 and optionally purifying or isolating the compound of interest.

The present invention is further illustrated by the following examples:

### EXAMPLES

The described invention combines the more direct approach of introducing multiple gRNA cassettes and the efficiency of resulting multiple genome modifications by connecting each new gRNA sequence to a standard component of a to-be-recombined expression vector functional in yeast. These standard DNA fragments would bear the sequences of components of a yeast expression vector (e.g. 2-micron, dominant resistance or auxotrophic marker, colour marker).

The gRNA sequences are introduced to the DNA fragments by overlap extension PCR using oligonucleotide primers of e.g. 60 nucleotides in length needing no additional costly purification methods at the oligonucleotide supplier. After transformation of four DNA fragments flanked on each side with individual guide sequences and subsequent in vivo recombination of the plasmid, correct transformants are selected based on complementation of an auxotrophic marker or antibiotics (facilitated by the presence of a marker on one of the DNA fragments) and fluoresense/colouration of the colony (facilitated by the presence of a colour marker on one of the DNA fragments). Correct transformants are screened on the targeted modifications by colony PCR, restriction analysis and/or sequencing of the target locus.

### Example 1

### Construction of Cas9-Expressing Pentose-Fermenting Yeast Strain

For demonstration of S. cerevisiae strain modification using a CRISPR-Cas9 system including a vector according to the present invention, the genetically modified strain BIE272 (described in US20140141473) was used. This strain is able to ferment both hexose sugars (glucose, mannose, galactose) and pentose sugars (xylose and arabinose) because of the introduction of heterologous utilization pathways. Prior to the introduction of a 4gRNA self-assembling vector system according to the invention (see below), BIE272 was modified to express the Streptococcus pyogenes Cas9 protein (DiCarlo et al., 2013). The Cas9 expression cassette was integrated in combination with the natMX marker on an integration locus on chromosome 14. The complete sequence of the integration fragment including flanks to the integration locus, Cas9 expression cassette and the natMX marker is included in here as SEQ ID NO 2.

### Example 2

### Description of the Standard Biobricks for 4gRNA-Vector Assembly in S. cerevisiae

For the expression of gRNA sequences into S. cerevisiae a gRNA expression cassette as previously described (DiCarlo et al., 2013) was used. The gRNA expression cassettes comprises the SNR52 promoter, the gRNA sequence consisting of the guide-sequence (crRNA; 20 nt) and the structural component, followed by the SUP4 terminator.

For the assembly of a episomally expressed plasmid in yeast there are certain elements which are required for the replication of the plasmid in yeast, or upon which selection can be exerted. The plasmid is assembled from 4 different polynucleotides (hereafter indicated as biobricks) each one corresponding to a polynucleotide consisting of a polynucleotide sequence to be extended in the overlap-extension PCR reaction described in Example 3.

Each biobrick is flanked:
on the 5′-end terminus by a fragment of the guide RNA expression cassette corresponding to the structural component of the gRNA and SUP4 terminator.
on the 3′-end terminus by a fragment of the guide RNA expression cassette corresponding to part of the SNR52 promoter for type III RNA polymerase transcription.

The assembly approach described in this example may be also compatible with hybrid gRNA platforms, like, for example, the Alt-R™ CRISPR-Cas9 System (Integrated DNA Technologies, Leuven, Belgium), by adapting the 5′ ends/termini of the BioBrick flanks replacing the structural component sequence by a crRNA sequence similar to a Alt-R™ CRISPR crRNA sequence that excludes the guide-sequence (also named protospacer element). In that case, the generic Alt-R™ CRISPR tracrRNA (Integrated DNA technologies, Leuven, Belgium) has to be included in the transformation or has to be part of one of the BioBricks to form a functional Alt-R™ crRNA:tracrRNA complex.

The biobricks (the polynucleotide sequences to be extended) used in the assembly comprise the following elements:

### Brick 1 (ORI-Yeast)

For the plasmid to be maintained in yeast an origin of replication (ORI) is necessary. For the efficient expression of gRNA in yeast an ORI which leads to high copy number of the resulting plasmid is highly desirable. Therefore, the 2-micron (2μ) plasmid sequence (Broach et al.) was selected as element for the plasmid in Brick 1 (SEQ ID NO. 3).

### Brick 2 (Color Marker)

For easy visual identification of correct transformants a chromogenic or fluorescent protein may be used. There are many varieties of chromogenic or fluorescent proteins such as Red Fluorescent Protein (RFP), Green Fluorescent Protein (GFP), mCherry, dsRed, etcetera. For Brick 2 an expression cassette of Dasher GFP (DNA2.0, Palo Alto, Calif., USA) flanked upstream by the S. cerevisae TDH3 promoter, and flanked downstream by the S. cerevisiae ADH1 terminator sequence (SEQ ID NO 4) was selected.

### Brick 3 (Dominant Resistance or Auxotrophic Marker)

To allow for selection of transformants any suitable markers suitable for S. cerevisiae may be used. One group of markers are the auxotrophic markers. Upon introduction or complementation of the gene sequence encoding the auxotrophic marker, a previously lacking enzymatic activity in a metabolic pathway towards an essential S. cerevisiae building block (e.g. adenine, leucine, uracil, histidine). Drawback is the need for an auxotrophic strain to be able to use the auxotrophic marker. Another group of markers which can be used in prototrophic strains is a dominant resistance marker (DRM). Upon transformation of the gene sequence of a DRM to a host cell gives rise to resistance to an added compound (e.g. antibiotic, antifungicide) in the growth medium whereas the untransformed host cell which did not express the gene sequence would remain sensitive and would not grow. Examples of functional DRMs in yeast are e.g. kanMX, hphMX, and natMX (Goldstein & McCusker, 1999). In this example, for Brick 3, kanMX was selected as dominant resistance marker in S. cerevisiae (SEQ ID NO 5).

Brick 4 (Bacterial ampR/kanR and ORI)

To enable E. coli-based plasmid amplification for sub-sequential use in another host organism, a biobrick containing a bacterial selection marker (ampR or kanR) and ORI (e.g. pUC) may be used. In this example, for Brick 4 the kanRgene, a bacterial selection marker, and pUC ORI were selected (SEQ ID NO 6).

Bricks 1, 2, 3, and 4 were produced synthetically (e.g. DNA2.0, Palo Alto, Calif., USA) and served as standard templates for the introduction of the 20 bp-long polynucleotide coding for the gRNA-sequences by overlap-extension PCR method.

### Example 3

### Using the 4gRNA Assembly System to Delete HXT Genes in BIE272

In this example the 4gRNA-vector assembly system was used to delete four hexose transporter gene clusters (HXT3-HXT6-HXT7 on chromosome IV, HXT5-HXT1-HXT4 on chromosome VIII, HXT2 on chromosome XIII, GAL2 on chromosome XII) in BIE272 rendering this pentose-fermenting strain unable to grow on pentose sugars since the pentose uptake capacity is eliminated with deletion of of the genes hxt1-hxt2-hxt3-hxt4-hxt5-hxt6-hxt7(indicated as hxt1-7 hereafter) and gal2 (Nijland et al., 2014). The gRNA sequences for HXT1 (to delete cluster HXT5-HXT1-HXT4 cluster), HXT2 (to delete HXT2), HXT3 (to delete cluster HXT3-HXT6-HXT7) and GAL2 (to delete GAL2) were selected on the DNA2.0 website (https:/www.dna20.come/eCommerce/cas9/input). The guide sequences with score 100 were selected for implementation into the 4gRNA assembly system.

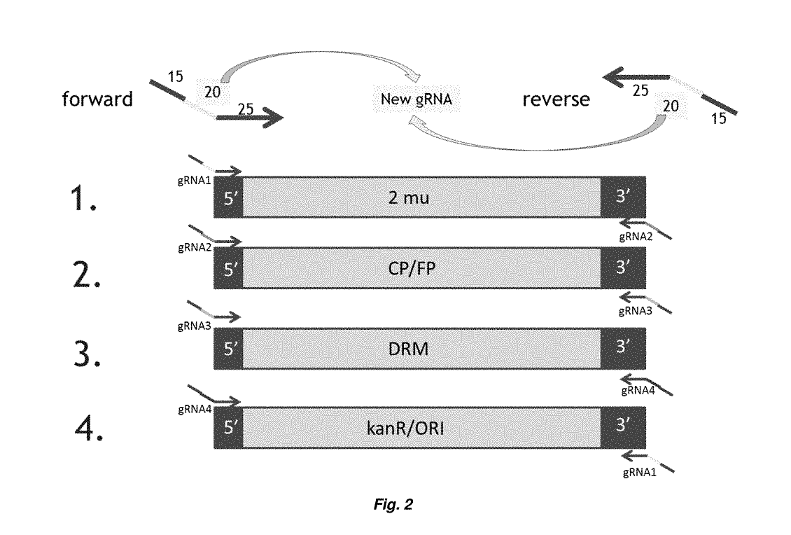

Extension of Biobricks with gRNA sequences targeting HXT genes by overlap extension PCR 20-bp polynucleotides coding for the four selected gRNA guide-sequences were added to both the 5′-terminus and the 3′-terminus of each of the Bricks 1 to 4 (previously described above, SEQ ID NOs: 3-6) by overlap extension PCR (see FIG. 2 for a schematic representation). The sequences of coding for the four guide-sequences were present in the primer sequences. Table 1, depicts to which Brick the different gRNA sequences were introduced.

TABLE 1
orientation of the gRNA sequences to the Bricks

| Biobrick | 5′-flank | 3′-flank |
|---|---|---|
| Brick 1 (2μ) | gRNA1 (HXT1) | gRNA2 (HXT2) |
| Brick 2 (GFP) | gRNA2 (HXT2) | gRNA3 (HXT3) |
| Brick 3 (kanMX) | gRNA3 (HXT3) | gRNA4 (GAL2) |
| Brick 4 (kanR/ORI) | gRNA4 (GAL2) | gRNA1 (HXT1) |

Generally, for extending the Biobricks with gRNA sequences, oligonucleotide primers consisted of the following parts going from 5′-end to 3′-end direction (see FIG. 2 for schematic representation):

a) forward primers (annealing to 5′ end of Biobrick)
15 nucleotides of the SNR52 promoter sequence directly upstream of the gRNA sequence
20 nucleotides of the gRNA sequence, and
25 nucleotides of the structural component directly downstream of the gRNA sequence, namely the nucleotides of the template Biobrick to which the oligonucleotide primer annealed

b) reverse primers (annealing to 3′end of Biobrick)
15 nucleotides of the structural component of the gRNA directly downstream of the gRNA sequence
20 nucleotides of the gRNA sequence, and
25 nucleotides of the SNR52 promoter sequence directly upstream of the gRNA sequence, namely the nucleotides of the template Biobrick to which the oligonucleotide annealed

Table 2 depicts the sequences in the different parts of the oligonucleotide primers (the 15, the 20, and the 25 nt part, respectively) and Table 3 depicts the full sequences of the oligonucleotide primers used in the OE-PCR reactions.

TABLE 2
standardized setup of oligonucleotide primers used in this example

| Primer | | | |
|---|---|---|---|
| orientation | 15 (5′→3′) | 20 (5′→3′) | 25 (5′→3′) |
| Forward | TGAAAGAT | NNNNNNNNNNNNNNNN | GTTTTAGAGCTAGAAATAGC |
| | AAATGAT | NNNN | AAGTT (SEQ ID NO: 117) |
| | (SEQ ID NO: | | |
| | 116) | | |
| Reverse | TTCTAGCTC | NNNNNNNNNNNNNNNN | ATCATTTATCTTTCACTGCG |
| | TAAAAC | NNNN | GAGAA (SEQ ID NO: 119) |
| | (SEQ ID NO: | | |
| | 118) | | |

TABLE 3
oligonucleotide primers used to fuse gRNA sequences to Biobricks for
HXT gene deletions

| | SEQ | |
|---|---|---|
| | ID | Sequence (5′ to 3′; in italics |
| primer | NO | and underlined the gRNA sequence is depicted) |
| gR1-HXT1-f | 7 | TGAAAGATAAATGATGTTGTAGTCAGCGCCTCTCTGTTTTAGAGCTAGA |
| | | AATAGCAAGTT |
| gR2-HXT2-f | 8 | TGAAAGATAAATGATTGGGTTAACCTTAGGACGTCGTTTTAGAGCTAGA |
| | | AATAGCAAGTT |
| gR3-HXT3-f | 9 | TGAAAGATAAATGATGGTGGCTAGTATTTGTTTCGGTTTTAGAGCTAGA |
| | | AATAGCAAGTT |
| gR4-GAL2-f | 10 | TGAAAGATAAATGATTCTAACTCTGCCTGGACGGCGTTTTAGAGCTAGA |
| | | AATAGCAAGTT |
| gR1-HXT1-r | 11 | TTCTAGCTCTAAAACAGAGAGGCGCTGACTACAACATCATTTATCTTTC |
| | | ACTGCGGAGAA |
| gR2-HXT2-r | 12 | TTCTAGCTCTAAAACGACGTCCTAAGGTTAACCCAATCATTTATCTTTC |
| | | ACTGCGGAGAA |
| gR3-HXT3-r | 13 | TTCTAGCTCTAAAACCGAAACAAATACTAGCCACCATCATTTATCTTTC |
| | | ACTGCGGAGAA |
| gR4-GAL2-r | 14 | TTCTAGCTCTAAAACGCCGTCCAGGCAGAGTTAGAATCATTTATCTTTC |
| | | ACTGCGGAGAA |

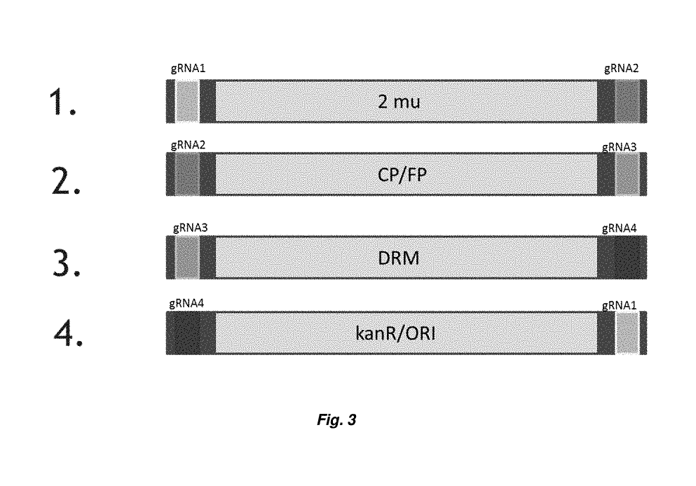

Using the primer design as displayed in Table 2 we have designed forward and reverse primers specifically comprising the gRNA sequences targeting HXT1, HXT2, HXT3, and GAL2, which were selected as explained above (see SEQ ID NO. 7 to 14). Bricks 1, 2, 3, 4 were extended with the gRNA sequences of gRNA1 (HXT1), gRNA2 (HXT2), gRNA3 (HXT3) or gRNA4 (GAL2) in the design as depicted in Table 1 and FIG. 2), by overlap extension PCR reaction. In Table 4, the specific combinations of forward primer, reverse primer and template Biobrick were given. PCR reactions were carried out using Phusion High-Fidelity DNA Polymerase (Thermoscientific Landsmeer, the Netherlands) according to the instructions of the supplier. As annealing temperature 60° C. was chosen. The resulting PCR products (hybrid linear polynucleotides) with SEQ ID NOs listed in Table 4 were subsequently purified using a standard PCR purification kit (Promega). DNA concentrations were measured on a Nanodrop device (Thermoscientific Landsmeer, the Netherlands). By this way 4 double-stranded DNA products were amplified corresponding to the hybrid linear polynucleotides (indicated in the examples as extended Biobricks), being 4 linear DNA elements to be used for yeast transformation and in-vivo assembly (see FIG. 3).

TABLE 4
primer combinations for PCR amplifications of gRNA fusion to Bricks 1-4

| | | Forward | Reverse | | | |
|---|---|---|---|---|---|---|
| | Template | primer | primer | | | PCR product |
| Brick | DNA | (SEQ ID NO) | (SEQ ID NO) | gRNA 5′ | gRNA 3′ | (SEQ ID NO) |
| 1 | 2μORI | 7 | 12 | HXT1 | HXT2 | 15 |
| 2 | GFP | 8 | 13 | HXT2 | HXT3 | 16 |
| 3 | kanMX | 9 | 14 | HXT3 | GAL2 | 17 |
| 4 | kanR/ORI | 10 | 11 | GAL2 | HXT1 | 18 |

### Co-Transformation of Extended Biobricks and Donor DNA Fragments and In Vivo Assembly of 4gRNA-Vector in Yeast

To enable the deletion of the four targeted HXT clusters after the targeting of the Cas9-gRNA complex four donor DNA fragments were acquired (IDT, Leuven, Belgium). The donor DNA fragments SEQ ID NO: 19 (HXT5-HXT1-HXT4-repair), SEQ ID NO: 20 (HXT2-repair), SEQ ID NO: 21 (HXT3-HXT6-HXT7-repair), SEQ ID NO: 22 (GAL2-repair) facilitate the repair of the double-stranded break made by Cas9 at the targeted loci of HXT1, HXT2, HXT3 or GAL2, respectively, by homologous recombination, thereby deleting the coding sequences of the targeted HXT genes.

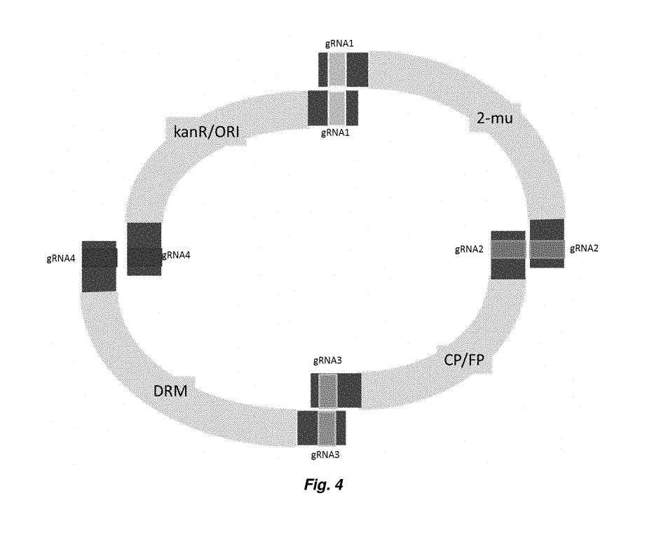

In six separate transformations, the Cas9-expressing BIE272 strain (described in Example 1) was transformed with the extended Biobricks (each transformation with a different concentration namely 31.25, 62.5, 125, 250, 500 and 1000 ng per extended Bricks) obtained above (SEQ ID NOs: 15 to 18), and the donor DNA fragments (1000 ng per donor DNA fragment; SEQ ID NO: 19 to 22). Yeast transformation was done according to the method described by Schiestl and Gietz (Current Genetics (1989), Volume 16, 339-346). 1000×-diluted suspensions of transformed cells were plated on rich growth agar medium (YePhM; 1% w/v yeast extract, 2% w/v phytone, 2% v/v maltose) supplemented with 200 mg/L G418. After 2-3 days of growth at 30° 0, colonies appeared on the selection plates. Upon transformation, the PCR products consisting of the 4 extended Bricks have to be assembled in the correct configuration in S. cerevisiae due to the flanking regions of homology present at the 5′- and 3-termini of each extended biobrick and which comprise the gRNA sequences (see FIG. 4 and SEQ ID NOs: 15 to 18). Plates were inspected on a blue light transilluminators for GFP-emitting colonies. Thirty-five colonies of which thirty, which remained GFP-emitting colonies after re-streaking, were selected and were re-streaked on yeast nitrogen-base (6.7 g/L yeast nitrogen base w/o amino acids, Sigma-Aldrich, Zwijndrecht) agar (15 g/L) medium supplemented with 2% maltose (YNB-maltose) or 2% xylose (YNB-xylose), and rich growth agar medium (YePhM; 1% w/v yeast extract, 2% w/v phytone, 2% v/v maltose) supplemented with 200 mg/L G418 (YePhM-G418). The restreaked colonies that were able to grow on YePhM-G418, in construct to the untransformed strain BIE272-Cas9, and emitted GFP fluorescence indicated the successful assembly of the extended Biobricks indicating the presence of Brick 3 (kanMX) due to the ability to grow on G418, the presence of Brick 2 (GFP) due to the emitted fluorescence, and the presence of Brick 1 (2μ ORI) due to the combined traits (growth on G418 and emitted fluorescence) which could only be possible by the replication of the plasmid in S. cerevisiae enabled by the 2μ ORI. The restreaked colonies were checked for the intended modifications of the HXT genes by diagnostic colony PCR. Four colonies out of 30 showed an impaired or absent growth profile on YNB-xylose while maintaining growth on YePhM-G418 and YNB-maltose indicating an effect on growth on xylose by the introduction of the transformed extended Biobricks and donor DNA fragments.

Cell material samples of the 30 restreaked colonies growing on YePhM-G418 and GFP-emitting fluorescence were resuspended in Y-PER™ (Yeast Protein Extraction Reagent, Life Technologies), according to manufacturer's recommendations, and subsequently boiled to lyse cells. PCR reactions were carried out using Phusion High-Fidelity DNA Polymerase (Thermoscientific Landsmeer, the Netherlands) according to the instructions of the supplier. Suitable oligonucleotide primers were used in the PCR reactions to show the deletions of the different HXT clusters.

In Table 5, the numbers of colonies per intended modification are shown.

TABLE 5
Number of colonies showing specific deletions of
HXT genes/cluster based on diagnostic colony PCR

| | Number of colonies with deletion |
|---|---|
| Deletion | (x out of 30) |
| HXT5-HXT1-HXT4 | 21 |
| HXT2 | 8 |
| HXT3-HXT6-HXT7 | 2 |
| GAL2 | 2 |

Based on these results it can be concluded that by using the 4 gRNA assembly system in BIE272 all intended modifications at the gene loci (HXT5-HXT1-HXT4, HXT2, HXT3-HXT6-HXT7 and GAL2) targeted by the expressed gRNA sequences could be conducted indicating succesfull assembly of plasmids expressing the gRNA sequences.

### Example 4

### Using the 4gRNA-Vector Assembly System to Introduce Three Expression Cassettes Encoding Together a Beta-Carotene Production Pathway at Three Different Loci

In this example, the 4gRNA-vector assembly system was used to target three intergenic, non-coding integration loci (INT1, INT59, YPRCtau3) in CEN.PK113-7D to integrate three individual expression casettes, encompassing the three genes of the beta-carotene pathway (crtE, crtYB and crtI from Xanthophyllomyces dendrorhous) with each casette targeted to a different integration locus. Upon successful integration and expression of all three genes in the Saccharomyces cerevisiae genome, successful transformants will produce colored carotenoid compounds resulting in yellow-, orange- or red-colored transformants (Verwaal et al., 2007). Coloring of the cells is a result of carotenoid production and can be achieved by expressing crtE, crtYB and crtI using promoters and terminators functional in S. cerevisiaeto express these genes (Verwaal et al., 2007). This example demonstrates that by using the 4gRNA-vector assembly method of the invention, the three beta-carotene pathway gene expression cassettes can be transformed to 3 different loci simultaneously, allowing multiplex genome engineering in the genomic DNA of S. cerevisiae, resulting in colored transformants, reflecting correctly edited cells.

### Construction of a CAS9-Expressing CEN.PK1 13-7D Strain

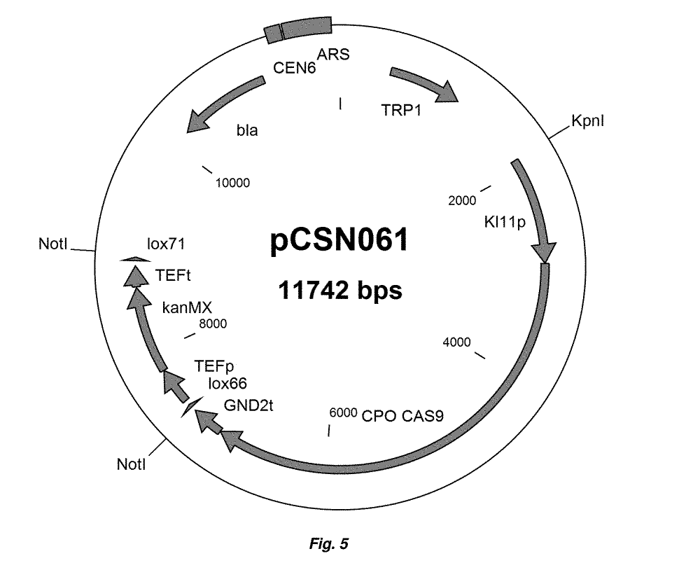

Vector pCSN061 (SEQ ID NO: 23, graphical depiction in FIG. 5) containing a CAS9 cassette was first transformed to S. cerevisiae strain CEN.PK113-7D (MATa URA3 HIS3 LEU2 TRP1 MAL2-8 SUC2) using the LiAc/salmon sperm (SS) carrier DNA/PEG method (Gietz and Woods, 2002). Strain CEN.PK113-7D is available from the EUROSCARF collection (http://www.euroscarf.de, Frankfurt, Germany) or from the Centraal Bureau voor Schimmelcultures (Utrecht, the Netherlands, entry number CBS 8340). The origin of the CEN.PK family of strains is described by van Dijken et al., 2000. In the transformation mixture 1 microgram of vector pCNSO61 was used. The transformation mixture was plated on YPD-agar (10 grams per litre of yeast extract, 20 grams per litre of peptone, 20 grams per litre of dextrose, 20 grams per litre of agar) containing 200 microgram (pIg) G418 (Sigma Aldrich, Zwijndrecht, the Netherlands) per ml. After two to four days of growth at 30° C. colonies appeared on the transformation plate. A yeast colony conferring resistance to G418 on the plate, now referred as strain CSN001, was inoculated on YPD-G418 medium (10 grams per litre of yeast extract, 20 grams per litre of peptone, 20 grams per litre of dextrose, 200 μg G418 (Sigma Aldrich, Zwijndrecht, the Netherlands) per ml).

### Extension of Biobricks with Guide Sequences by Overlap Extension PCR

In this example Brick 3b (SEQ ID NO: 24) was used bearing the natMX dominant resistance marker because the kanMX marker was used to express Cas9 from episomal plasmid pCSN061 (FIG. 5). Different from Example 3, in this example two of the four Bricks, Brick 1 and Brick 2, were extended on both the 5′-terminus and the 3′-terminus with 20-bp polynucleotides coding the selected guide sequences (indicated in Table 6 and in this example as gRNA's). The other two Bricks, Brick 3b and Brick 4, were extended with selected gRNA sequences on either the 5′-terminus or the 3′-terminus, respectively (see Table 6) using the primers as given in Table 7. The other termini (i.e. 3′-terminus of Brick 3b and 5′-terminus of Brick 4) were not extended with gRNA sequences, but on each Brick with non-coding 25 nucleotide sequences that are homologous to each other to facilitate in vivo recombination.

TABLE 6
Orientation of the gRNA sequences to the Bricks

| Biobrick | 5′-terminus | 3′-terminus |
|---|---|---|
| Brick 1 (2μ) | gRNA1 (INT59) | gRNA2 (YPRCtau3) |
| Brick 2 (GFP) | gRNA2 (YPRCtau3) | gRNA3 (INT1) |
| Brick 3b (natMX) | gRNA3 (INT1) | No gRNA |
| Brick 4 (kanR/ORI) | No gRNA | gRNA1 (INT59) |

TABLE 7
Oligonucleotide primers used to fuse guide sequences (or
overlapping sequences between Brick 3b and Brick 4) to
Biobricks to target integration loci INT59, YPRCtau3, and INT1.

| | SEQ | Sequence (5' to 3'; in italics and underlined the gRNA |
|---|---|---|
| | ID | sequence is depicted; grey boxed text is hybridizing |
| primer | NO | nucleotides to respective biobrick) |
| gR1-INT59-f | 25 | TGAAAGATAAATGATCAGAAAACTCTTAGCTTTTCCGTTTTAGAGCTAGAAATAGCAAGTT |
| gR2-YPRc-f | 26 | TGAAAGATAAATGATCCAATATGGTATGCCGAGTCTGTTTTAGAGCTAGAAATAGCAAGTT |
| gR3-INT1-f | 27 | TGAAAGATAAATGATCTATTAGAACCAGGGAGGTCCGTTTTAGAGCTAGAAATAGCAAGTT |
| Brick4_to3-f | 28 | 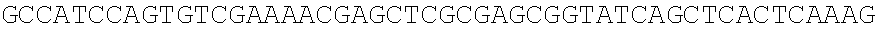 |
| gR1-INT59-r | 29 | TTCTAGCTCTAAAACGGAAAAGCTAAGAGTTTTCTGATCATTTATCTTTCACTGCGGAGAA |
| gR2-YPRc-r | 30 | TTCTAGCTCTAAAACAGACTCGGCATACCATATTGGATCATTTATCTTTCACTGCGGAGAA |
| gR3-INT1-r | 31 | TTCTAGCTCTAAAACGGACCTCCCTGGTTCTAATAGATCATTTATCTTTCACTGCGGAGAA |
| Brick3_to4-r | 32 | 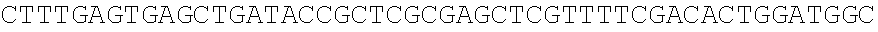 |

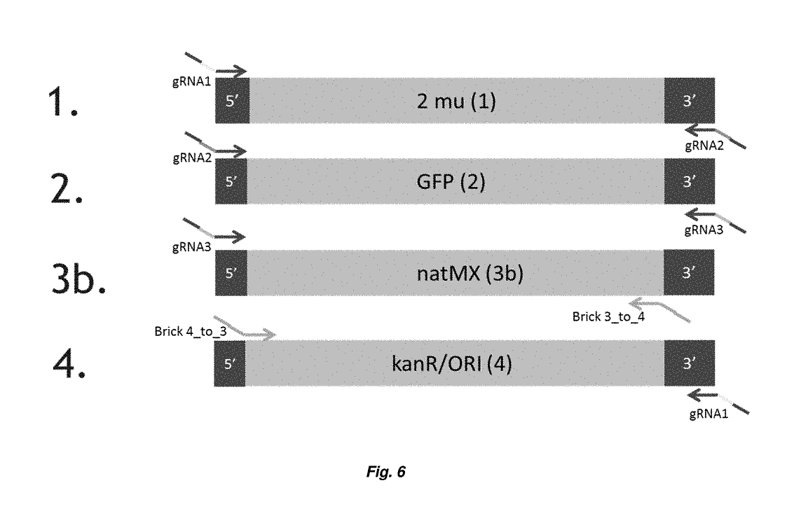

Using the primer design as displayed in Table 2 we have designed forward and reverse primers specifically comprising the gRNA sequences targeting the INT59, YPRCtau3 and INT1 loci that were selected for CRISPR/CAS9-mediated integration of crtYB, crtI and crtE expression cassettes as explained above. Bricks 1, 2, 3, 4 were extended with the gRNA sequences of gRNA1 (INT59), gRNA2 (YPRCtau3), or gRNA3 (INT1) by overlap extension PCR reaction in the design as depicted in Table 8. The position for gRNA4 (as depicted in FIG. 6) was omitted. Instead, a reverse 50 nt primer was designed hybridizing to a 25 nt sequence directly upstream of the SNR52 promoter on Brick 3b (primer Brick3_to4-r, SEQ ID NO: 32), and a 50 nt forward primer was designed hybridizing to a 25 nt sequence directly downstream of the SUP4 terminator on Brick 4 (primer Brick4_to3-f, SEQ ID NO: 28). The remaining 25 nt flanks on each primer facilitated a 50 nt overlap between the resulting PCR-amplification products using Brick 3b (PCR product SEQ ID NO: 35) and Brick 4 (PCR product SEQ ID NO: 36) as templates.

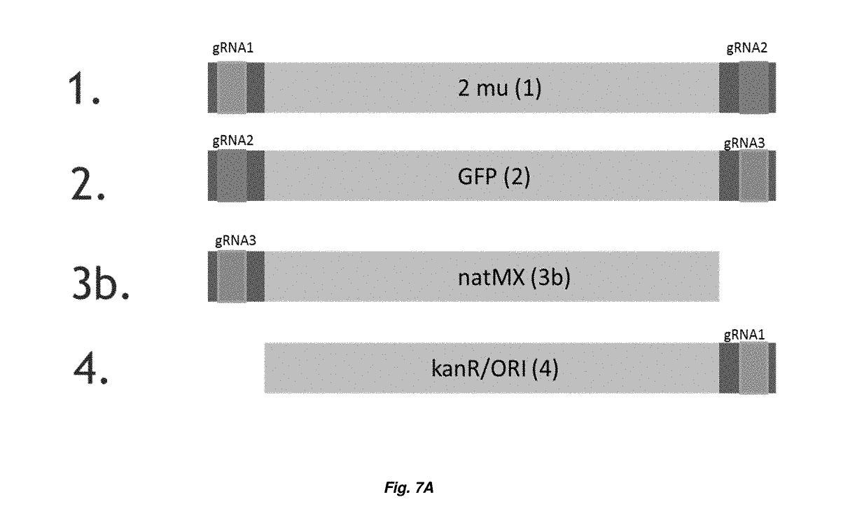

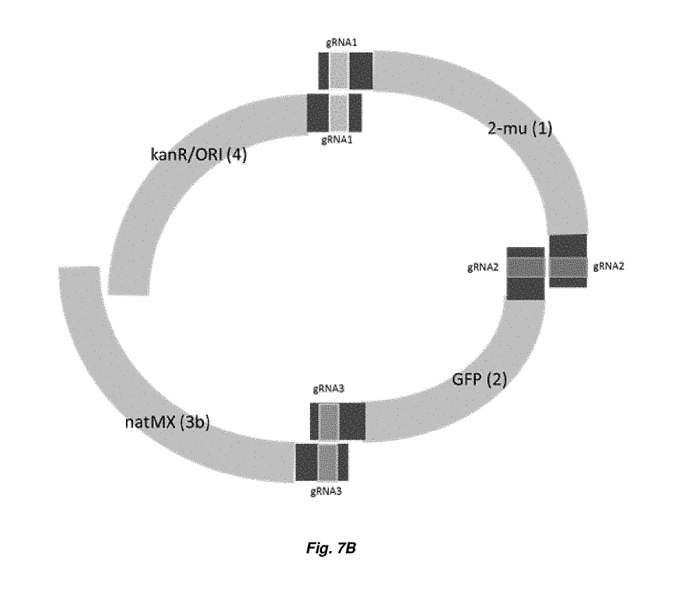

In Table 8, the specific combinations of forward primer, reverse primer and template Biobrick are provided. PCR reactions were carried out using Phusion High-Fidelity DNA Polymerase (Thermoscientific Landsmeer, the Netherlands) according to the instructions of the supplier. As annealing temperature 60° C. was chosen. The resulting PCR products (hybrid linear polynucleotides) with SEQ ID NOs listed in Table 8 were subsequently purified using a standard PCR purification kit (Promega). DNA concentrations were measured on a Nanodrop device (Thermoscientific Landsmeer, the Netherlands). By this way 4 double-stranded DNA products were amplified corresponding to the hybrid linear polynucleotides (indicated in the examples as extended Biobricks), being 4 linear DNA elements to be used for yeast transformation as depicted in FIG. 7A and in vivo assembly into a circular yeast expression vector (see FIG. 7B) resulting in SEQ ID NO: 33-36 (see Table 8).

TABLE 8
Primer combinations for PCR amplifications of gRNA fusion to Bricks 1, 2, 3b and 4

| | | Forward | Reverse | | | |
|---|---|---|---|---|---|---|
| | Template | primer | primer | | | PCR product |
| Brick | DNA | (SEQ ID NO) | (SEQ ID NO) | gRNA 5′ | gRNA 3′ | (SEQ ID NO) |
| 1 | 2μORI | 25 | 30 | INT59 | YPRcτ3 | 33 |
| 2 | GFP | 26 | 31 | YPRcτ3 | INT1 | 34 |
| 3b | natMX | 27 | 32 | INT1 | No gRNA | 35 |
| 4 | kanR/ORI | 28 | 29 | No gRNA | INT59 | 36 |

### Co-Transformation of Extended Biobricks Together with Donor DNA Fragments, and In Vivo Assembly of 4gRNA-Vector and Multiplex Integration of Three Donor DNA Expression Cassettes in Yeast

To enable multiplex integration of three beta-carotene pathway genes expression cassettes encoding crtE, crtYB and crtI, at the three different integration loci being INT1, INT59 and YPRCtau3, the CRISPR/CAS9 system combined with the 4gRNA-vector assembly system was used. The INT1 integration site is located at the non-coding region between NTR1 (YOR071c) and GYP1 (YOR070c) located on chromosome XV. The INT59 (in this example named INT2) integration site is a non-coding region between SRP40 (YKR092C) and PTR2 (YKR093W) located on chromosome XI. The YPRCtau3 (in this example named INT3) integration site is a Ty4 long terminal repeat, located on chromosome XVI, and has been described by Flagfeldt et al. (2009).

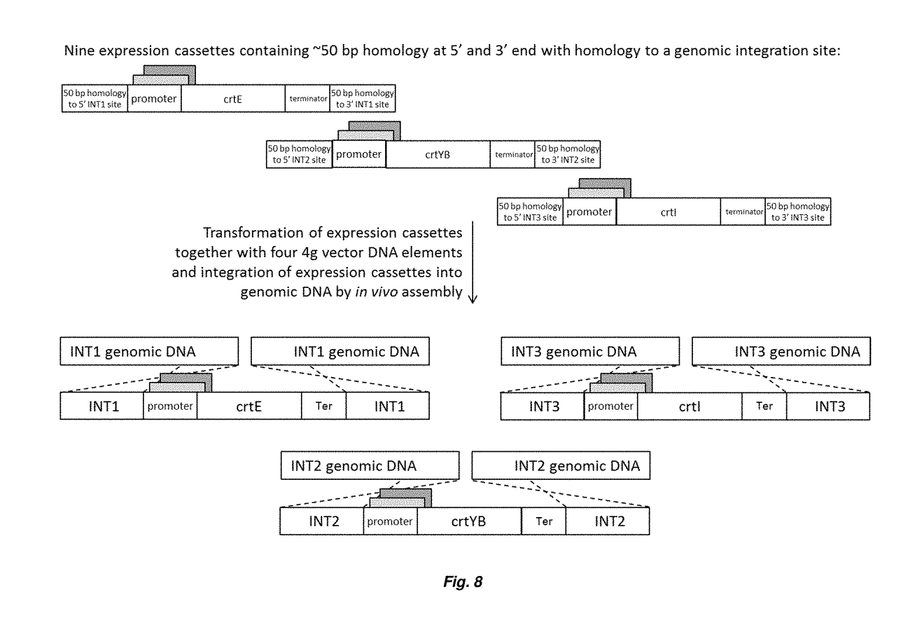

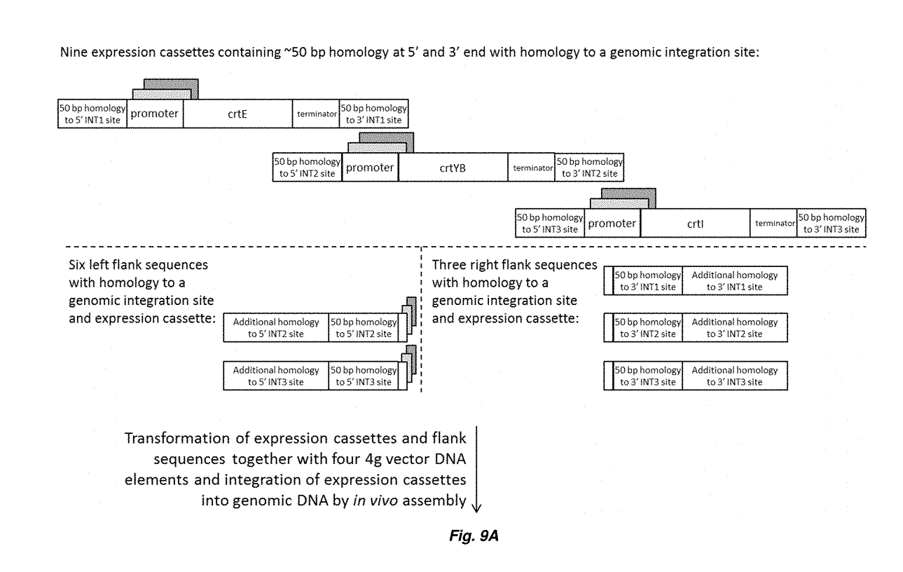

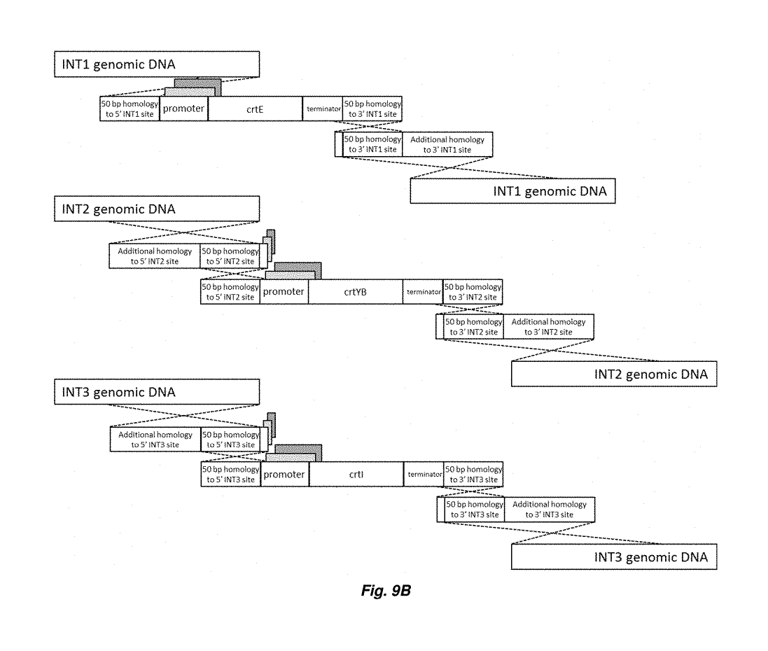

First, donor DNA required for this example was obtained as described below. This donor DNA was subsequently used in two different transformation experiments, which basically differ in the length of DNA homology flanks sequences added in the transformation experiment, as described below and depicted in FIG. 8 and FIGS. 9A and B.

### Donor DNA Expression Cassette Sequences

Carotenoid gene donor DNA expression cassettes, being different crtE, crtYB and crtI expression cassettes, of which the sequences are set out in SEQ ID NO: 47 to SEQ ID NO: 55 (Table 9), were obtained by PCR and were used as donor DNA expression cassettes that were integrated into genomic DNA using the approaches described in this example. PCR fragments of the donor DNA expression cassette and flank sequences were generated by PCR using Phusion DNA polymerase (New England Biolabs, USA) according to manufacturer's instructions. In case of the expression cassettes of the beta-carotene pathway genes, the synthetic DNA provided by DNA2.0 (Menlo Park, USA) was used as a template in the PCR reaction, using the specific forward and reverse primer combinations depicted in Table 9, wherein the PCR reaction is performed according to standard methods known to those skilled in the art. For example, in order to obtain the PCR fragment set out in SEQ ID NO: 47, the synthetic DNA construct SEQ ID NO: 38 provided by DNA2.0 was used as a template, using primer sequences set out in SEQ ID NO: 56 and SEQ ID NO: 57. In total, nine different donor DNA sequences containing the carotenoid gene expression cassettes were generated by PCR, as set out in SEQ ID NO: 47; 48; 49; 50; 51; 52; 53; 54 and 55. All donor DNA PCR fragments were purified using the NucleoSpin Gel and PCR Clean-up kit (Machery-Nagel, distributed by Bioké, Leiden, the Netherlands) according to manufacturer's instructions. DNA concentrations were measured on a Nanodrop device (Thermoscientific Landsmeer, the Netherlands).

Carotenoid gene expression cassettes used in transformation experiment #1 (FIG. 8, Table 9) were composed of the following elements: at the 5′ and 3′ positions of the DNA sequence, approximately 50 basepair (bp) flank sequences were present that contain homology with the desired genomic integration site (INT1, INT2 or INT3). In this example INT1 is the INT1 integration site, INT2 is the INT59 integration site, and INT3 is the YPRCtau3 integration site. The presence of flank sequences allowed introduced of carotenoid expression cassettes into the genomic DNA. As a result, different donor DNA fragments assembled into the genomic DNA at different desired location, as is depicted in FIG. 8. A promoter sequence, which can be homologous (i.e. from S. cerevisiae) or heterologous (e.g. from Kluyveromyces lactis) and a terminator sequences derived from S. cerevisiae, were used to control the expression of the beta-carotene pathway genes crtE, crtYB or crtI. As described in Table 9 the promoters were expected to have different expression strengths, resulting in low, medium or high expression levels of crtE, crtYB or crtI. As shown in Example 9 of PCT/EP2016/050136, a combination of crtE, crtYB and crtI expression cassettes with low strength promoters gave the lowest production levels of total carotenoids, medium strength promoters gave higher production levels of total carotenoids and strong promoters gave the highest levels of total carotenoids. The crtE, crtYB and crtI nucleotide sequences were codon pair optimized for expression in S. cerevisiae as described in WO2008/000632.

TABLE 9
Overview of different donor DNA expression cassette sequences used in transformation
experiment #1 and transformation experiment #2. Under description, the following
elements are indicated: The promoter including the relative expected expression strengths
(Low p = low strength promoter, Med p = medium strength promoter, Strong
p = high strength promoter). The ORF name, crtE, crtYB or crtl, and the terminator
sequence. This table includes the SEQ ID NO's of the primers used to obtain the
donor DNA expression cassette sequences by amplification by PCR. INT1: INT1 integration
site. INT2: INT59 integration site. INT3: YPRCtau3 integration site.

| | | | | Synthetic | | |
|---|---|---|---|---|---|---|
| Donor DNA | | | | DNA | | |
| (expression | Promoter | | | template | Forward | Reverse |
| cassettes) | strength | Description | Targeting to | for PCR | primer | primer |
| SEQ ID | L | Homology to INT1 - | INT1 | SEQ ID | SEQ ID | SEQ ID |
| NO: 47 | | Low p (KITDH2p) - | | NO: 38 | NO: 56 | NO: 57 |
| | | crtE - ScTDH3t - | | | | |
| | | Homology to INT1 | | | | |
| SEQ ID | M | Homology to INT1 - | INT1 | SEQ ID | SEQ ID | SEQ ID |
| NO: 48 | | Med p (KIPGK1p) - | | NO: 39 | NO: 58 | NO: 57 |
| | | crtE - ScTDH3t - | | | | |
| | | Homology to INT1 | | | | |
| SEQ ID | S | Homology to INT1 - | INT1 | SEQ ID | SEQ ID | SEQ ID |
| NO: 49 | | Strong p (ScFBA1p) - | | NO: 40 | NO: 59 | NO: 57 |
| | | crtE - ScTDH3t - | | | | |
| | | Homology to INT1 | | | | |
| SEQ ID | L | Homology to INT2 - | INT2 | SEQ ID | SEQ ID | SEQ ID |
| NO: 50 | | Low p (KIYDR1p) - | | NO: 41 | NO: 60 | NO: 61 |
| | | crtYB-ScPDC1t - | | | | |
| | | Homology to INT2 | | | | |
| SEQ ID | M | Homology to INT2 - | INT2 | SED ID | SEQ ID | SEQ ID |
| NO: 51 | | Med p (KITEF2p) - | | NO: 42 | NO: 62 | NO: 61 |
| | | crtYB - ScPDC1t - | | | | |
| | | Homology to INT2 | | | | |
| SEQ ID | S | Homology to INT2 - | INT2 | SEQ ID | SEQ ID | SEQ ID |
| NO: 52 | | Strong p (ScTEF1p) - | | NO: 43 | NO: 63 | NO: 61 |
| | | crtYB - ScPDC1t - | | | | |
| | | Homology to INT2 | | | | |
| SEQ ID | L | Homology to INT3 - | INT3 | SEQ ID | SED ID | SEQ ID |
| NO: 53 | | Low p (ScPRE3p) - | | NO: 44 | NO: 64 | NO: 65 |
| | | crtl - ScTAL1t - | | | | |
| | | Homology to INT3 | | | | |
| SEQ ID | M | Homology to INT3 - | INT3 | SEQ ID | SED ID | SEQ ID |
| NO: 54 | | Med p (ScACT1p) - | | NO: 45 | NO: 66 | NO: 65 |
| | | crtl - ScTAL1t - | | | | |
| | | Homology to INT3 | | | | |
| SEQ ID | S | Homology to INT3 - | INT3 | SEQ ID | SED ID | SEQ ID |
| NO: 55 | | Strong p (KIENO1p) - | | NO: 46 | NO: 67 | NO: 65 |
| | | crtl - ScTAL1t - | | | | |
| | | Homology to INT3 | | | | |

### Donor DNA Flank Sequences

Nine donor DNA flank sequences PCR fragments containing homology with the genomic integration site and the donor DNA expression cassette were included in transformation experiment 2 (FIG. 9, Table 10). The donor DNA flanks sequences were obtained as follows: Genomic gDNA was isolated from the yeast strain CEN.PK113-7D (MATa URA3 HIS3 LEU2 TRP1 MAL2-8 SUC2) using the lithium acetate SDS method (Lõoke et al., 2011). This genomic DNA was used as a template to obtain the PCR fragments that were used as donor for DNA flanking sequences (comprising the overlap with the genomic DNA for genomic integration), using the specific forward and reverse primer combinations depicted in Table 10. PCR fragments of the donor DNA flank sequences were generated by PCR using Phusion DNA polymerase (New England Biolabs, USA) according to manufacturer's instructions. For example, in order to obtain the Right Flank (RF) INT1 (sequence of the PCR fragment is set out in SEQ ID NO: 68), genomic DNA isolated from strain CEN.PK113-7D was used as a template, using primer sequences set out in SEQ ID NO: 77 and SEQ ID NO: 78. For example, in order to obtain the Left Flank (LF) INT2 sequence with additional homology to the KIYDR2 promoter used for expression of crtYB (sequence of the PCR fragment is set out in SEQ ID NO: 69), genomic DNA isolated from strain CEN.PK113-7D was used as a template, using primer sequences set out in SEQ ID NO: 79 and SEQ ID NO: 80. Six different donor LF DNA flank sequences were generated by PCR, as set out in SEQ ID NO: 69; 70; 71; 73; 74 and 75. Three different RF donor DNA flank sequences were generated by PCR, as set out in SEQ ID NO: 68; 72 and 76. All donor DNA PCR fragments were purified using the NucleoSpin Gel and PCR Clean-up kit (Machery-Nagel, distributed by Bioké, Leiden, the Netherlands) according to manufacturer's instructions. DNA concentrations were measured on a Nanodrop device (Thermoscientific Landsmeer, the Netherlands).

The donor DNA flank sequences contain homology with sequences present in the integration sites and donor DNA expression cassette sequences and allows in vivo recombination between integration sites and donor DNA in yeast as depicted in FIG. 9. The LF sequences contain at their 5′ end homology with the INT2 or INT3 integration site (part of the INT2 and INT3 sequence is present on the crtYB or crtI expression cassette PCR fragment), and contain at their 3′ end homology with the specific promoters used in the crtYB and crtI expression cassettes (depicted in FIG. 9). The RF sequences contain at their 5′ end homology with the specific terminators used in the crtE, crtYB or crtI expression cassettes and contain at the 3′ end homology with the INT1, INT2 or INT3 integration site (part of the INT1, INT2 or INT3 sequence is present on the crtE, crtYB and crtI expression cassette PCR fragment respectively; depicted in FIG. 9).

TABLE 10
Overview of different donor DNA flank sequences additionally used in transformation
experiment #2. The homology with genomic DNA indicates the overlap of the
flank sequence with the genomic integration site in basepairs (bp).

| | | | | Homology | | |
|---|---|---|---|---|---|---|
| Donor DNA | | | | with | | |
| (flank | | | | genomic | Forward | Reverse |
| sequences) | Flank | Targeting to | Description | DNA (bp) | primer | primer |
| SEQ ID | RF (3′) | INT1 | RF INT1 (all | 524 | SEQ ID | SEQ ID |
| NO: 68 | | | crtE cassettes) | | NO: 77 | NO: 78 |
| SEQ ID | LF (5′) | INT2 | LF INT2 (crtYB | 322 | SEQ ID | SEQ ID |
| NO: 69 | | | KIYDR1p) | | NO: 79 | NO: 80 |
| SEQ ID | LF (5′) | INT2 | LF INT2 (crtYB | 322 | SEQ ID | SEQ ID |
| NO: 70 | | | KITEF2p) | | NO: 79 | NO: 81 |
| SEQ ID | LF (5′) | INT2 | LF INT2 (crtYB | 322 | SEQ ID | SEQ ID |
| NO: 71 | | | ScTEF1p) | | NO: 79 | NO: 82 |
| SEQ ID | RF (3′) | INT2 | RF INT2 (all | 496 | SEQ ID | SEQ ID |
| NO: 72 | | | crtYB cassettes) | | NO: 83 | NO: 84 |
| SEQ ID | LF (5′) | INT3 | LF INT3 (crtl | 599 | SEQ ID | SEQ ID |
| NO: 73 | | | KIYDR1p) | | NO: 85 | NO: 86 |
| SEQ ID | LF (5′) | INT3 | LF INT3 (crtl | 599 | SEQ ID | SEQ ID |
| NO: 74 | | | KITEF2p) | | NO: 85 | NO: 87 |
| SEQ ID | LF (5′) | INT3 | LF INT3 (crtl | 599 | SEQ ID | SEQ ID |
| NO: 75 | | | ScTEF1p) | | NO: 85 | NO: 88 |
| SEQ ID | RF (3) | INT3 | RF INT3 (all | 625 | SEQ ID | SEQ ID |
| NO: 76 | | | crtl cassettes) | | NO: 89 | NO: 90 |

### Transformation Experiments

S. cerevisiae strain CSN001, which is described above under “Construction of a CAS9-expressing CEN.PK113-7D strain”, was grown and transformed with the four linear 4gRNA-vector DNA elements outlined in FIG. 7 (SEQ ID NO: 33, SEQ ID NO: 34, SEQ ID NO: 35, SEQ ID NO: 36) in two different transformation experiments. In addition, in transformation experiment #1, nine different donor DNA elements comprising beta-carotene expression cassettes (Table 10, Table 11) were transformed as graphically depicted in FIG. 8. In addition, in transformation experiment #2, in total eighteen donor DNA elements were transformed, including nine different beta-carotene expression cassettes, six different left flank and three different right flank sequences (Table 10, Table 11) as graphically depicted in FIG. 9. Donor DNA expression cassette PCR fragments (see Table 9 for SEQ ID NO's) were used in the transformation experiment #1 and transformation experiment #2. Donor DNA flank PCR fragments (see Table 11 for SEQ ID NO's) were only used in transformation experiment #2. Additional flank sequences were added in transformation experiment #2 to determine whether additional homology of donor DNA by including separate larger flank sequences allows for a higher genome editing efficiency.

TABLE 11
Overview of donor DNA and 4gRNA-vector DNA elements used in transformation
experiment #1 and transformation experiment #2.

| Transformation | | 4gRNA-vector | Donor DNA | |
|---|---|---|---|---|
| experiment | Description experiment | DNA elements | expression cassettes | Flanks sequences |
| #1 | Nine crt expression | SEQ ID NO: 33 | SEQ ID NO: 47 | |
| | cassettes (PCR | SEQ ID NO: 34 | SEQ ID NO: 48 | |
| | fragments) containing | SEQ ID NO: 35 | SEQ ID NO: 49 | |
| | different low-, | SEQ ID NO: 36 | SEQ ID NO: 50 | |
| | medium- and high- | | SEQ ID NO: 51 | |
| | strength promoters | | SEQ ID NO: 52 | |
| | targeted to INT1, INT2 | | SEQ ID NO: 53 | |
| | and INT3 | | SEQ ID NO: 54 | |
| | | | SEQ ID NO: 55 | |
| #2 | Nine crt expression | SEQ ID NO: 33 | SEQ ID NO: 47 | SEQ ID NO: 68 |
| | cassettes (PCR | SEQ ID NO: 34 | SEQ ID NO: 48 | SEQ ID NO: 69 |
| | fragments) containing | SEQ ID NO: 35 | SEQ ID NO: 49 | SEQ ID NO: 70 |
| | different low-, | SEQ ID NO: 36 | SEQ ID NO: 50 | SEQ ID NO: 71 |
| | medium- and high- | | SEQ ID NO: 51 | SEQ ID NO: 72 |
| | strength promoters | | SEQ ID NO: 52 | SEQ ID NO: 73 |
| | together with six LF | | SEQ ID NO: 53 | SEQ ID NO: 74 |
| | and three RF PCR | | SEQ ID NO: 54 | SEQ ID NO: 75 |
| | fragments targeted to | | SEQ ID NO: 55 | SEQ ID NO: 76 |
| | INT1, INT2 and INT3, | | | |
| | together with six LF | | | |
| | and three RF PCR | | | |
| | fragments | | | |

Strain CSN001 was transformed using the LiAc/salmon sperm (SS) carrier DNA/PEG method (Gietz and Woods, 2002). 200 ng of each of the nine beta-carotene pathway donor DNA expression cassettes was included in transformation experiments #1 and #2. 250 ng of each 4gRNA-vector part was included in transformation experiments #1 and #2. 100 ng of each of the nine donor DNA flanks sequences was included in transformation experiment #2. The transformation mixtures were plated on YPD-agar (10 grams per litre of yeast extract, 20 grams per litre of peptone, 20 grams per litre of dextrose, 20 grams per litre of agar) containing 200 μg nourseothricin (NatMX, Jena Bioscience, Germany) and 200 μg G418 (Sigma Aldrich, Zwijndrecht, the Netherlands) per ml. After two to four days of growth at 30° C., colonies appeared on the transformation plates.

After transformation, the total number of colonies on a transformation plate were counted. The transformants were colored and/or non-colored. In case of colored transformants, the crtE, crtYB and crtI expression cassettes were successfully integrated into the genomic DNA of the yeast cells enabling production of carotenoids (Verwaal et al., 2007; PCT/EP2016/050136). In case of non-colored transformants, crtE, crtYB and crtI expression cassettes were not successfully integrated into the genomic DNA of the yeast cells. The percentage of successfully engineered cells, i.e. transformants that have integrated the crtE, crtYB and crtI expression cassettes into genomic DNA, was calculated by dividing the number of colored transformants by the number of total transformants. The results are shown in Table 12.

TABLE 12
Percentage colored cells obtained in the two different
transformation experiments plated on YPD (2%) + G418 +
NatMX agar plates (double selection) to allow selection
on both the CAS9 and guide RNA containing vectors.

| Transformation | | % Colored |
|---|---|---|
| experiment | Description experiment | cells |
| #1 | Donor DNA with ~50 bp homology flanks | 37% |
| #2 | Donor DNA with ~50 bp homology flanks | 59% |
| | and additional donor DNA flank sequences | |

Based on these results it can be concluded that the 4gRNA-vector assembly system can be used to integrate multiple expression cassettes within the genomic DNA of yeast at different integration sites, and demonstrate that the 4gRNA-vector was successfully assembled in yeast, allowing expressing of multiple functional guide RNA's. These results also demonstrate that including LF and RF flank sequences, creating a higher homology of the donor DNA expression cassettes with the intended chromosomal integration site, increases the genome editing efficiency from 37% to 59%.

This transformation and 4gRNA-vector assembly system in combination with transformation of different donor DNA expression cassettes also allows to obtain a large variety in different transformants that can be obtained. Because nine different donor DNA expression cassettes were included in the transformation, the beta-carotene pathway expression cassettes can be combined to 27 different combinations, resulting in different expression levels of crtE, crtYB and crtI, because of the different promoters used in the expression constructs (Table 13). This resulted in transformants with different yellow, orange or red colors that were obtained after transformation (data not shown), indicative of different combinations of expression cassettes integrated into the genomic DNA of the transformants, and these different transformants are expected to produce different levels of carotenoids. This approach is beneficial for screening purposes, where a large number of different transformants could be screened for different properties, for example for different production levels of a compound or compounds of interests, in this case for different carotenoid production levels.

TABLE 13
27 different combinations can be made when nine different
crt expression cassettes (see Table 11) were transformed.

| Combination | Promoter strength | Promoter | ORF |
|---|---|---|---|
| 1 | L | KITDH2 | crtE |
| | L | KIYDR1 | crtYB |
| | L | ScPRE3 | crtI |
| 2 | L | KITDH2 | crtE |
| | L | KIYDR1 | crtYB |
| | M | ScTAL1 | crtI |
| 3 | L | KITDH2 | crtE |
| | L | KIYDR1 | crtYB |
| | H | KIENO1 | crtI |
| 4 | L | KITDH2 | crtE |
| | M | KITEF2 | crtYB |
| | L | ScPRE3 | crtI |
| 5 | L | KITDH2 | crtE |
| | M | KITEF2 | crtYB |
| | M | ScTAL1 | crtI |
| 6 | L | KITDH2 | crtE |
| | M | KITEF2 | crtYB |
| | H | KIENO1 | crtI |
| 7 | L | KITDH2 | crtE |
| | H | ScTEF1 | crtYB |
| | L | ScPRE3 | crtI |
| 8 | L | KITDH2 | crtE |
| | H | ScTEF1 | crtYB |
| | M | ScTAL1 | crtI |
| 9 | L | KITDH2 | crtE |
| | H | ScTEF1 | crtYB |
| | H | KIENO1 | crtI |
| 10 | M | KIPGK1 | crtE |
| | L | KIYDR1 | crtYB |
| | L | ScPRE3 | crtI |
| 11 | M | KIPGK1 | crtE |
| | L | KIYDR1 | crtYB |
| | M | ScTAL1 | crtI |
| 12 | M | KIPGK1 | crtE |
| | L | KIYDR1 | crtYB |
| | H | KIENO1 | crtI |
| 13 | M | KIPGK1 | crtE |
| | M | KITEF2 | crtYB |
| | L | ScPRE3 | crtI |
| 14 | M | KIPGK1 | crtE |
| | M | KITEF2 | crtYB |
| | M | ScTAL1 | crtI |
| 15 | M | KIPGK1 | crtE |
| | M | KITEF2 | crtYB |
| | H | KIENO1 | crtI |
| 16 | M | KIPGK1 | crtE |
| | H | ScTEF1 | crtYB |
| | L | ScPRE3 | crtI |
| 17 | M | KIPGK1 | crtE |
| | H | ScTEF1 | crtYB |
| | M | ScTAL1 | crtI |
| 18 | M | KIPGK1 | crtE |
| | H | ScTEF1 | crtYB |
| | H | KIENO1 | crtI |
| 19 | H | ScFBA1 | crtE |
| | L | KIYDR1 | crtYB |
| | L | ScPRE3 | crtI |
| 20 | H | ScFBA1 | crtE |
| | L | KIYDR1 | crtYB |
| | M | ScTAL1 | crtI |
| 21 | H | ScFBA1 | crtE |
| | L | KIYDR1 | crtYB |
| | H | KIENO1 | crtI |
| 22 | H | ScFBA1 | crtE |
| | M | KITEF2 | crtYB |
| | L | ScPRE3 | crtI |
| 23 | H | ScFBA1 | crtE |
| | M | KITEF2 | crtYB |
| | M | ScTAL1 | crtI |
| 24 | H | ScFBA1 | crtE |
| | M | KITEF2 | crtYB |
| | H | KIENO1 | crtI |
| 25 | H | ScFBA1 | crtE |
| | H | ScTEF1 | crtYB |
| | L | ScPRE3 | crtI |
| 26 | H | ScFBA1 | crtE |
| | H | ScTEF1 | crtYB |
| | M | ScTAL1 | crtI |
| 27 | H | ScFBA1 | crtE |
| | H | ScTEF1 | crtYB |
| | H | KIENO1 | crtI |

### Example 5

Using the 1gRNA-Vector Assembly System to Delete Chromosomal DNA

In this example, a 1gRNA-vector assembly system was used to target a GFP expression cassette to the INT1 locus and to achieve deletion of approximately 1000 basepairs (1 kilobase pairs (kb)) or approximately 10,000 basepairs (10 kb) of genomic DNA at the INT1 locus. The INT1 integration site is located at the non-coding region between NTR1 (YOR071c) and GYP1 (YOR070c) located on chromosome XV.

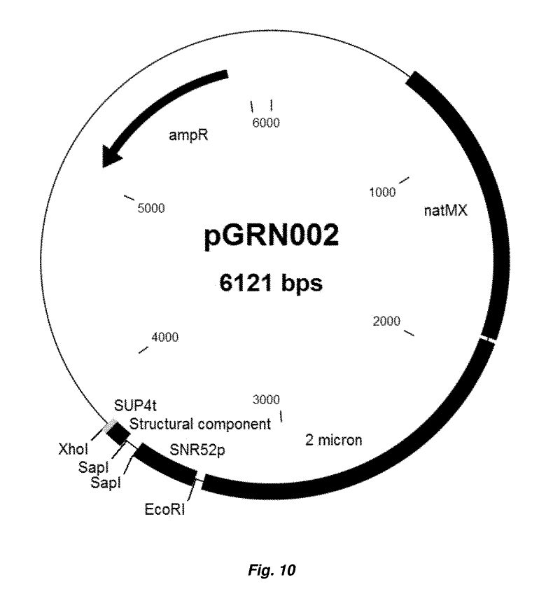

pRN1120 (SEQ ID NO: 91) is a yeast multi-copy vector (2 micron) that contains a functional NatMX marker cassette conferring resistance against nourseothricin. The backbone of this vector is based on pRS305 (Sikorski and Hieter, 1989), including a functional 2 micron ORI sequence and a functional NatMX (nourseothricin resistance) marker cassette (see http://www.euroscarf.de). The SapI restriction site was removed from the pRN1120 backbone by PCR using the primers set out in SEQ ID NO: 92 and SEQ ID NO: 93, changing the SapI restriction site from GCTCTTC to cCTCTTC. Recircularization of the intermediate PCR fragment without a SapI site was performed using the KLD enzyme mix of the Q5 site directed mutagenesis kit (New England Biolabs, supplied by Bioké, Leiden, the Netherlands. Catno. E0554S) according to the supplier's manual. The resulting plasmid was digested by EcoRI and XhoI. By Gibson assembly a gBlock containing amongst others a SNR52 promoter, a guide RNA structural component and a SUP4 terminator sequence (Integrated DNA Technologies, Leuven, Belgium), sequence provided in SEQ ID NO: 94, was added to the pRN1120-SapI backbone. Gibson assembly is performed using Gibson Assembly HiFi 1 Step Kit (SGi-DNA, La Jolla, Calif., USA. Catno. GA1100-50) according to supplier's manual. The resulting plasmid was designated pGRN002 (SEQ ID NO: 95, FIG. 10), containing a SNR52 promoter, a guide RNA structural component and a SUP4 terminator sequence in which the guide sequence can be cloned/assembled by making use of the SapI sites. For the expression of gRNA sequences in S. cerevisiae, a gRNA expression cassette as previously described by DiCarlo et al., 2013 was used. The gRNA expression cassettes comprises the SNR52 promoter, the gRNA sequence consisting of the guide-sequence or guide sequence, and the guide RNA structural component, followed by the SUP4 terminator. The guide sequence used in this example will guide the CAS9 protein to the INT1 location in genomic DNA of S. cerevisiae.

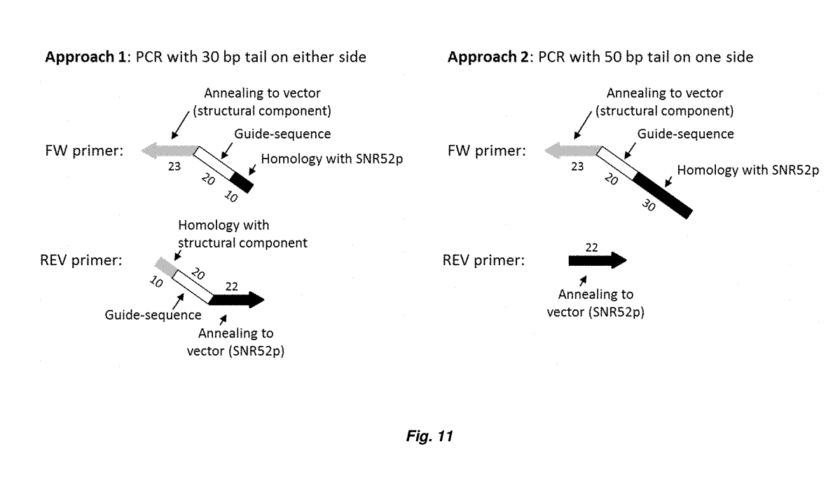

Vector pGRN002 was used as a template in two different PCR reactions using oligonucleotide primers indicated in Table 14 and graphically depicted in FIG. 11. The oligonucleotide primers consist of the following parts going from the 5′-end to the 3′-end direction:

a) Forward primer used to obtain PCR fragment #1 (SEQ ID NO: 96):
10 nucleotides of the SNR52 promoter sequence directly upstream of the guide sequence,
20 nucleotides of the guide sequence, and
23 nucleotides of the structural component of the gRNA directly downstream of the guide sequence, to which the oligonucleotide annealed in the pGRN002 vector.

b) Reverse primer used to obtain PCR fragment #1 (SEQ ID NO: 97):
10 nucleotides of the structural component of the gRNA directly downstream of the guide sequence,
20 nucleotides of the guide sequence, and
22 nucleotides of the SNR52 promoter sequence directly upstream of the guide sequence, to which the oligonucleotide annealed in the pGRN002 vector.

c) Forward primer used to obtain PCR fragment #2 (SEQ ID NO: 99):
30 nucleotides of the SNR52 promoter sequence directly upstream of the guide sequence,
20 nucleotides of the guide sequence, and
23 nucleotides of the structural component of the gRNA directly downstream of the guide sequence, to which the oligonucleotide annealed in the pGRN002 vector.

d) Reverse primer used to obtain PCR fragment #2 (SEQ ID NO: 100):
22 nucleotides of the SNR52 promoter sequence directly upstream of the guide sequence, to which the oligonucleotide annealed in the pGRN002 vector.

The Q5 DNA polymerase (part of the Q5® High-Fidelity 2X Master Mix, New England Biolabs, supplied by Bioké, Leiden, the Netherlands. Catno. M0492S) was used in the PCR reaction, which was performed according to manufacturer's instructions.

TABLE 14
Primer combinations for PCR amplifications to obtain 1gRNA-
vector PCR fragment #1 and PCR fragment #2.

| | | Forward | Reverse | | PCR |
|---|---|---|---|---|---|
| | | primer | primer | | product |
| | Template | (SEQ | (SEQ | Guide | (SEQ |
| Description | DNA | ID NO:) | ID NO:) | sequence | ID NO:) |
| 1gRNA PCR | pGRN002 | 96 | 97 | INT1 | 98 |
| fragment #1 | | | | | |
| 1gRNA PCR | pGRN002 | 99 | 100 | INT1 | 101 |
| fragment #2 | | | | | |

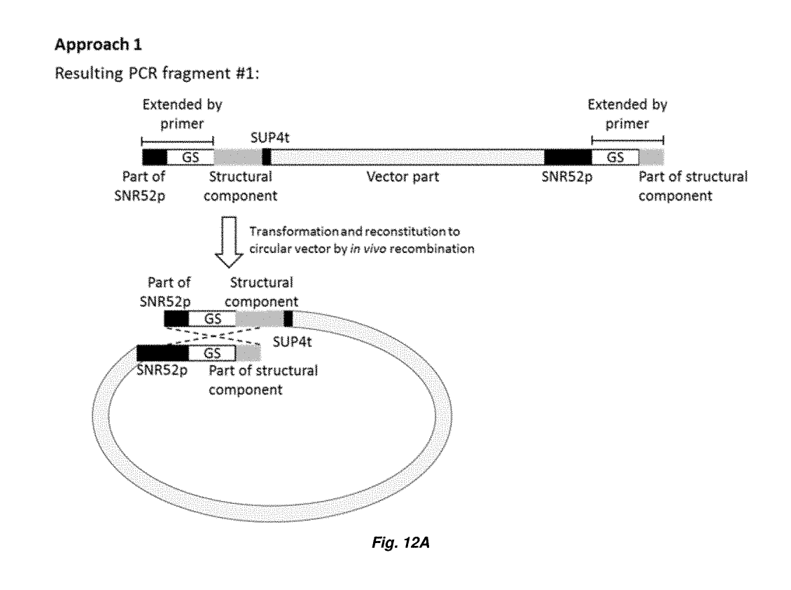

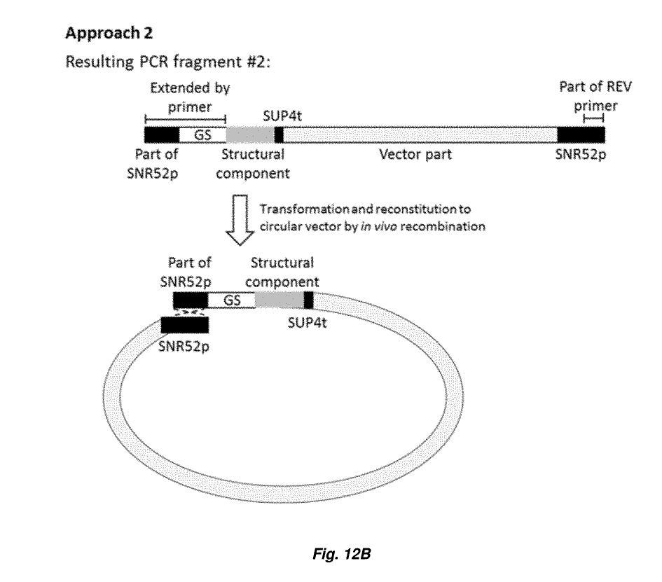

The resulting PCR fragment #1 contains on the 5′-end/terminus a part of the guide RNA expression cassette corresponding to the SNR52 promoter and the guide sequence, and on the 3′-end/terminus a fragment of the guide RNA expression cassette corresponding to the guide sequence and the structural component of the gRNA (FIG. 12, SEQ ID NO: 98). The resulting PCR fragment #2 contains on the 5′-end/terminus a part of the guide RNA expression cassette corresponding to the SNR52 promoter and the guide sequence, and on the 3′-end/terminus a fragment of the guide RNA expression cassette corresponding to the SNR52 promoter (FIG. 12). The presence of homologous sequences allows in vivo recombination in S. cerevisiae, resulting in a circular vector as graphically depicted in FIG. 12, encoding a functional guide RNA. The NatMX marker present on the circular vector can be used for selection transformants on nourseothricin. PCR fragment #1 was used for 1gRNA-vector assembly approach 1, where homology for in vivo recombination was obtained by part of the SNR52 promoter, guide sequence and part of the guide RNA structural component sequences. PCR fragment #2 was used for 1gRNA-vector assembly approach 2, where homology for in vivo recombination was obtained by part of the SNR52 promoter sequence only.

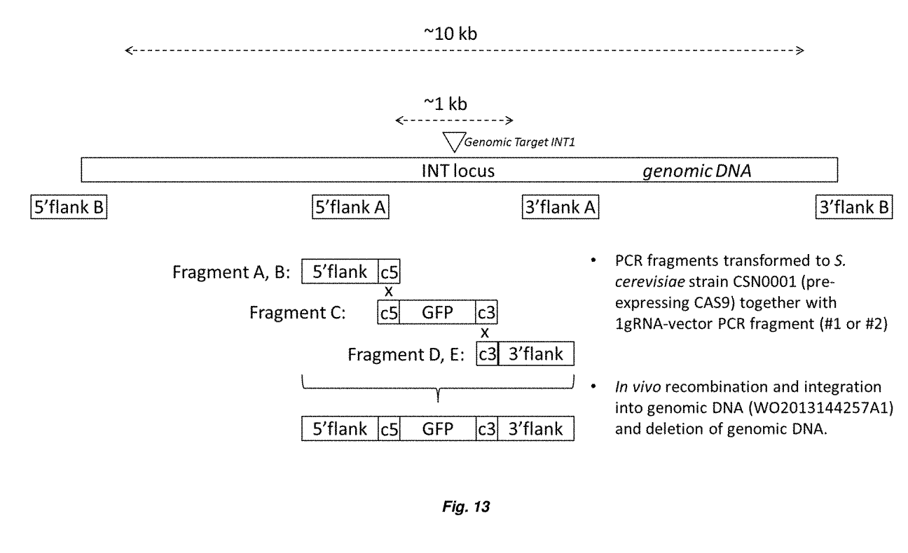

To achieve deletion up to approximately 1 kb pr 10 kb around the INT1 locus in the genomic DNA of yeast, PCR fragment #1 or PCR fragment #2 were transformed together with donor DNA sequences (left flank, GFP expression cassette, right flank, Table 15 and Table 16 to strain CSN001, which is pre-expressing CAS9 from a single copy KanMX containing vector (see Example 4). A schematic representation is shown in FIG. 13. Transformations were performed using the LiAc/SS carrier DNA/PEG method (Gietz and Woods, 2002). Deletion of 1 kb of genomic DNA around the INT1 integration site (approximately 500 bp upstream, approximately 500 bp downstream of the INT1 genomic target) or deletion of 10 kb of genomic DNA around the INT1 integration site (approximately 5 kb upstream, approximately 5 kb downstream of the INT1 genomic target) was expected to result in viable transformants, because no essential genes were fully or partially removed from the genomic DNA (source Saccharomyces genome database, http://www.yeastgenome.org/).

Deletion of 1 kb of genomic DNA is exemplified below and is depicted in FIG. 13: PCR fragment A (SEQ ID NO: 104), containing 450 bp homology with genomic DNA (5′flank A), was generated using the oligonucleotide sequences as set out in SEQ ID NO: 102 and SEQ ID NO: 103 and using a gBlock (SEQ ID NO: 104) as template (IDT, Leuven, Belgium). PCR fragment C (SEQ ID NO: 109), containing green fluorescent protein (GFP) expression cassette expressed from a S. cerevisiae TDH3 promoter and S. cerevisiae ENO1 terminator, was generated using the oligonucleotide sequences as set out in SEQ ID NO: 107 and SEQ ID NO: 108, using SEQ ID NO: 109 as template (a synthetic DNA cassette synthesized by DNA 2.0, Menlo Park, Calif., USA). PCR fragment D (SEQ ID NO: 112), containing 581 bp homology with genomic DNA (3′flank A), was generated using the oligonucleotide sequences as set out in SEQ ID NO: 110 and SEQ ID NO: 111 using genomic DNA isolated from S. cerevisiae strain CEN.PK113-7D as template (genomic DNA was isolated according to the method described by Lõoke et al., 2011). PCR reactions were performed by methods known by the person skilled in the art. Due to the presence of connector sequences, the 3′ part of PCR fragment A has homology with the 5′ part of PCR fragment C, and the 5′ part of PCR fragment D has homology with the 3′ part of fragment C, which allows homologous recombination into the genome of the yeast Saccharomyces cerevisiae (see FIG. 13) as is described in WO2013144257A1. Because CAS9 was targeted to the INT1 sequence present in the genomic DNA, a double strand break was introduced. The presence of homologous sequences will promote homologous recombination, and thus repair of the double stranded break and integration of the donor DNA sequences.

Deletion of 10 kb of genomic DNA is exemplified below and is depicted in FIG. 13: PCR fragment B (SEQ ID NO: 106), containing 450 bp homology with genomic DNA (5′flank B), was generated using the oligonucleotide sequences as set out in SEQ ID NO: 105 and SEQ ID NO: 103 and using a gBlock (SEQ ID NO: 107) as template (IDT, Leuven, Belgium). PCR fragment C (SEQ ID NO: 109), containing green fluorescent protein (GFP) expression cassette expressed from a S. cerevisiae TDH3 promoter and S. cerevisiae ENO1 terminator, was generated using the oligonucleotide sequences as set out in SEQ ID NO: 107 and SEQ ID NO: 108, using SEQ ID NO: 109 as template (a synthetic DNA cassette synthesized by DNA 2.0, Menlo Park, Calif., USA). PCR fragment E (SEQ ID NO: 115), containing 607 bp homology with genomic DNA (3′flank B), was generated using the oligonucleotide sequences as set out in SEQ ID NO: 113 and SEQ ID NO: 114 using genomic DNA isolated from S. cerevisiae strain CEN.PK113-7D as template (genomic DNA was isolated according to the method described by Lõoke et al., 2011). PCR reactions were performed by methods known by the person skilled in the art. Due to the presence of connector sequences, the 3′ part of PCR fragment B has homology with the 5′ part of PCR fragment C, and the 5′ part of PCR fragment E has homology with the 3′ part of fragment C, which allows homologous recombination into the genome of the yeast Saccharomyces cerevisiae (see FIG. 13) as is described in WO2013144257A1. Because CAS9 was targeted to the INT1 sequence present in the genomic DNA, a double strand break was introduced. The presence of homologous sequences will promote homologous recombination, and thus repair of the double stranded break and integration of the donor DNA sequences.

All donor DNA PCR fragments were purified using the NucleoSpin Gel and PCR Clean-up kit (Machery-Nagel, distributed by Bioké, Leiden, the Netherlands) according to manufacturer's instructions.

TABLE 15
Overview of PCR primers used to generate the PCR fragments
(left flank (LF) GFP expression cassette, right flank
(RF)), used to delete 1 kb or 10 kb of genomic DNA surrounding
the INT1 integration site (see FIG. 13).

| | | | Forward | Reverse | PCR |
|---|---|---|---|---|---|
| | | | primer | primer | product |
| | | | (SEQ | (SEQ | (SEQ |
| Name | Description | Template | ID NO:) | ID NO:) | ID NO:) |
| Fragment A | LF 1 kb | gBlock | 102 | 103 | 104 |
| | deletion | | | | |
| Fragment B | LF 10 kb | gBlock | 105 | 103 | 106 |
| | deletion | | | | |
| Fragment C | GFP | Synthetic | 107 | 108 | 109 |
| | expression | gene | | | |
| | cassette | | | | |
| Fragment D | RF 1 kb | Genomic | 110 | 111 | 112 |
| | deletion | DNA | | | |
| Fragment E | RF 10 kb | Genomic | 113 | 114 | 115 |
| | deletion | DNA | | | |

Four transformation experiments were performed in Saccharomyces cerevisiae strain CSN0001 (Example 4), according to the combinations of donor DNA and the 1gRNA-vector component as presented in Table 16. Transformation was performed using the LiAc/SS carrier DNA/PEG method (Gietz and Woods, 2002). Transformation mixtures were plated on YPD-agar (10 grams per litre of yeast extract, 20 grams per litre of peptone, 20 grams per litre of dextrose, 20 grams per litre of agar) containing 200 μg G418 (Sigma Aldrich) and 200 μg nourseothricin (NatMX, Jena Bioscience, Germany) per ml. KanMX conferred resistance to transformants containing vector pCSN061, expressing CAS9. NatMX conferred resistance to transformants containing the in vivo assembled circular vector of 1gRNA-vector approach 1 and 1gRNA-vector approach 2. After two to four days of growth at 30° C., colonies appeared on the transformation plates.

TABLE 16
PCR fragments included in the transformation experiments.

| Trans- | | | | | |
|---|---|---|---|---|---|
| forma- | Descrip- | 1gRNA- | Left | GFP | Right |
| tion | tion | vector | Flank | cassette | Flank |
| #1 | 1 kb | 1gRNA | Frag- | Frag- | Frag- |
| | deletion, | PCR | ment A | ment C | ment D |
| | approach 1 | fragment #1 | (SEQ ID | (SEQ ID | (SEQ ID |
| | | (SEQ ID | NO: 104) | NO: 109) | NO: 112) |
| | | NO: 98) | | | |
| #2 | 1 kb | 1gRNA | Frag- | Frag- | Frag- |
| | deletion, | PCR | ment A | ment C | ment D |
| | approach 2 | fragment #2 | (SEQ ID | (SEQ ID | (SEQ ID |
| | | (SEQ ID | NO: 104) | NO: 109) | NO: 112) |
| | | NO: 101) | | | |
| #3 | 10 kb | 1gRNA | Frag- | Frag- | Frag- |
| | deletion, | PCR | ment B | ment C | ment E |
| | approach 1 | fragment #1 | (SEQ ID | (SEQ ID | (SEQ ID |
| | | (SEQ ID | NO: 106) | NO: 109) | NO: 115) |
| | | NO: 98) | | | |
| #4 | 10 kb | 1gRNA | Frag- | Frag- | Frag- |
| | deletion, | PCR | ment B | ment C | ment E |
| | approach 2 | fragment #2 | (SEQ ID | (SEQ ID | (SEQ ID |
| | | (SEQ ID | NO: 106) | NO: 109) | NO: 115) |
| | | NO: 101) | | | |

Transformation of fragment A (5′flank A-Con5), fragment C (Con5-GFP expression cassette-Con3) and fragment D (Con3-3′flank A) resulted in the introduction of the GFP expression cassette and deletion of approximately 1 kb of the genomic DNA sequence. Transformation of fragment B (5′flank B-Con5), fragment C (Con5-GFP expression cassette-Con3) and fragment E (Con3-3′flank B) resulted in the introduction of the GFP expression cassette and deletion of approximately 10 kb of the genomic DNA sequence.

By UV light (Qpix 450 Colony Picker—Molecular devices LLC) a discrimination was made between green fluorescent colonies, indicating GFP integration, and white colonies, indicating no GFP integration, that appeared on the plates. The total number white and green fluorescent colonies on a transformation plate were counted. In case of green fluorescent transformants, the donor DNA was successfully integrated into the genomic DNA of the yeast cells. In The percentage of successfully engineered cells, i.e. transformants that have integrated the GFP expression cassette and flank sequences into genomic DNA, was calculated by dividing the number of green fluorescent transformants by the number of total transformants (Table 17).

TABLE 17
Results of the transformation experiments.

| | | | Total | |
|---|---|---|---|---|
| | | Number of | number | % Edited |
| Trans- | | fluorescent | of trans- | trans- |
| formation | Description | transformants | formants | formants |
| #1 | 1 kb deletion, | 8 | 9 | 89 |
| | approach 1 | | | |
| #2 | 1 kb deletion, | 8 | 9 | 89 |
| | approach 2 | | | |
| #3 | 10 kb deletion, | 2 | 3 | 67 |
| | approach 1 | | | |
| #4 | 10 kb deletion, | 4 | 5 | 80 |
| | approach 2 | | | |

The results of the transformation experiments (Table 17) demonstrate that the two different 1gRNA-vector approaches as depicted FIG. 12 resulted in obtaining GFP fluorescent strains, indicating that both 1 gRNA-vector approaches can be used for CRISPR-CAS9 mediated genome engineering experiments.

This experiment clearly shows that one can apply the 1 gRNA system in a fast, cheap and flexible way to create functional gRNA expression vectors by OE-PCR making use of short oligonucleotide primers and in-vivo recombination, using a standardized basic vector element that already contains all parts of an autonomously replicating vector and the structural parts of a gRNA sequence and control sequences which allow expression of the gRNA in the host cell, but without the guide sequence.

This method could also be applied to create linear autonomously replicating vectors, by using two fragments with OE-PCR at both left and right flanks to be assembled in a 1gRNA linear vector or optionally in a multiple gRNA linear vector.

### REFERENCES

Aleksenko and Clutterbuck. Fungal Genet. Biol. 1997 21: 373-397. Autonomous plasmid replication in Aspergillus nidulans: AMA1 and MATE elements.
Bao et al., 2015, ACS Synth Biol, volume 4 pp. 585-94. Homology-Integrated CRISPR-Cas (HI-CRISPR) System for One-Step Multigene Disruption in Saccharomyces cerevisiae.
Barnes et al., siRNA as a molecular tool for use in Aspergillus niger (2008) Biotechnology Letters 30 (5): 885-890.
Becker and Guarente, In Abelson, J. N. and Simon, M. I., editors, Guide to Yeast Genetics and Molecular Biology, Methods in Enzymology, Volume 194, 182-187, Academic Press, Inc., New York.
Beetham P R, Kipp P B, Sawycky X L, Arntzen C J and May G D. PNAS 1999, 96, 8774-8778. A tool for functional plant genomics: Chimeric RNA/DNA oligonucleotides cause in vivo gene-specific mutations.
Broach et al., 1991 In: Broach, J R.; Pringle, J R.; Jones, E W., editors. The Molecular Biology of the Yeast Saccharomyces. Genome Dynamics, Protein Synthesis and Energetics. Cold Spring Harbor Laboratory Press; Cold Spring harbor, New York. pp. 287-331
Burke D T, et al. Cloning of large segments of exogenous DNA into yeast by means of artificial chromosome vectors. Science 236: 806-812, 1987
Christianson T W, Sikorski R S, Dante M, Shero J H, Hieter P. Gene. 1992 Jan. 2; 110(1):119-22. Multifunctional yeast high-copy-number shuttle vectors.
Crook N C, Schmitz A C, Alper H S. ACS Synth Biol. 2014 16; 3(5):307-13. Optimization of a yeast RNA interference system for controlling gene expression and enabling rapid metabolic engineering.
DiCarlo J E, Norville J E, Mali P, Rios X, Aach J, Church G M. Nucleic Acids Res. 2013 April; 41(7):4336-43. Genome engineering in Saccharomyces cerevisiae using CRISPR-Cas systems.
Dong C, Beetham P, Vincent K and Sharp P. 2006 Plant Cell Rep 25: 457-465. Oligonucleotide-directed gene repair in wheat using a transient plasmid repair assay system.
J. A. Doudna, E. Charpentier Science (2104) 346: 1258096 DOI:10.1126/science.1258096. The new frontier of genome engineering with CRISPR-Cas9.
Durai S, Mani M, Kandavelou K, Wu J, Porteus M, Chandrasegaran S. Nucleic Acids Res 2005 33 (18): 5978-90. Zinc finger nucleases: custom-designed molecular scissors for genome engineering of plant and mammalian cells.
Finbarr Hayes (2003). “Chapter 1—The Function and Organization of Plasmids”. In Nicola Casali, Andrew Presto. E. Coli Plasmid Vectors: Methods and Applications. Methods in Molecular Biology, Vol. 235. Humana Press. pp. 1-5. ISBN 978-1-58829-151-6.
Flagfeldt D B, Siewers V, Huang L, Nielsen J. Yeast. 2009 October; 26(10):545-51. Characterization of chromosomal integration sites for heterologous gene expression in Saccharomyces cerevisiae.
Gaj T, Gersbach, C and Barbas C. Trends in Biotechnology, 2013, Vol. 31, No. 7 397-405. ZFN, TALEN, and CRISPR/Cas-based methods for genome engineering.
Gao Y and Zhao Y. J Integr Plant Biol. 2014 April; 56(4):343-9. Self-processing of ribozyme-flanked RNAs into guide RNAs in vitro and in vivo for CRISPR-mediated genome editing.
Gems D., Johnstone I. L., Clutterbuck A. J. Gene 1991 98(1):61-67. An autonomously replicating plasmid transforms Aspergillus nidulans at high frequency.
Gietz R D, Woods R A. Methods Enzymol. 2002; 350:87-96. Transformation of yeast by lithium acetate/single-stranded carrier DNA/polyethylene glycol method.
Goldstein, A. L., and McCusker, J. H. Yeast 1999. 15, 1541-15. Three new dominant drug resistance cassettes for gene disruption in Saccharomyces cerevisiae.
Guilinger J P, Thompson D B, Liu D R. Nat Biotechnol. 2014 577-582. Fusion of catalytically inactive Cas9 to FokI nuclease improves the specificity of genome modification.
Güldener, U., Heck, S., Fiedler, T., Beinhauer, J., and Hegemann, J. H. Nucleic Acids Research 1996. 24, 2519-2524. A new efficient gene disruption cassette for repeated use in budding yeast.
Heckman K. L., Pease L. R. Nature Protocols 2007 2(4): 924-932, Gene splicing and mutagenesis by PCR-driven overlap extension
Horton R. M., Hunt H. D., Ho S. N., Pullen K., Pease L. R. Gene 1989 77: 61-68, Engineering bybrid genes without the use of restriction enzymes: gene splicing by overlap extension
Horwitz A. A., Walter J. M., Schubert M. G., Kung S. H., Hawkings K., Platt D. M., Hernday A. D., Mahatdejkul-Meadows T., Szeto W., Chandran S. S., Newman J. D., Cell Systems 2015 1: 1-9, http://dx.doi.org/10.1016/j.cels.2015.02.001. Efficient Multiplexed Integration of Synergistic Alleles and Metabolic Pathways in Yeasts via CRISPR-Cas.
Hsu P D, Lander E S, Zhang F. Cell. 2014 Jun. 5; 157(6):1262-78. Development and applications of CRISPR-Cas9 for genome engineering.
Ito et al., 1983, Journal of Bacteriology 153: 163.
Jacobs J Z, Ciccaglione K M, Tournier V, Zaratiegui M. Nat Commun. 2014 Oct. 29; 5:5344. Implementation of the CRISPR-Cas9 system in fission yeast.
Jakočiūnas et al., 2015, ACS Synth Biol, http://dx.doi.org/10.1021/acssynbio.5b00007. CasEMBLR: Cas9-Facilitated Multiloci Genomic Integration of in Vivo Assembled DNA Parts in Saccharomyces cerevisiae.
Jørgensen T R, Park J, Arentshorst M, van Welzen A M, Lamers G, Vankuyk P A, Damveld R A, van den Hondel C A, Nielsen K F, Frisvad J C, Ram A F. Fungal Genet Biol. 2011 May; 48(5):544-53. The molecular and genetic basis of conidial pigmentation in Aspergillus niger.
Kleinstiver B P, Pattanayak V, Prew M S, Tsai S Q, Nguyen N T, Zheng Z, Joung J K, Nature. 2016 Jan. 28; 529(7587):490-5. High-fidelity CRISPR-Cas9 nucleases with no detectable genome-wide off-target effects.
Kornberg R. Trends in Cell Biology 1999 9 (12): M46 Eukaryotic transcriptional control.
Kuijpers et al. Microbial Cell Factories 2013, 12:47. A versatile, efficient strategy for assembly of multi-fragment expression vectors in Saccharomyces cerevisiae using 60 bp synthetic recombination sequences.
Larson, M. H.; Gilbert, L. A.; Wang, X; Lim, W. A.; Weissman, J. S.; Qi, L. S. Nature Protocols 2013 8 (11) 2180-96. CRISPR interference (CRISPRi) for sequence-specific control of gene expression.
LBoke M, Kristjuhan K, Kristjuhan A. Biotechniques. 2011 May; 50(5):325-8. Extraction of genomic DNA from yeasts for PCR-based applications.
Mali P, Yang L, Esvelt K M, Aach J, Guell M, DiCarlo J E, Norville J E, Church G M. Science. 2013 Feb. 15; 339(6121):823-6. RNA-guided human genome engineering via Cas9.
Mans et al., 2015, FEMS Yeast Res, volume 15, doi: 10.1093/femsyr/fov004. CRISPR/Cas9: a molecular Swiss army knife for simultaneous introduction of multiple genetic modifications in Saccharomyces cerevisiae
Marck C, Kachouri-Lafond R, Lafontaine I, Westhof E, Dujon B, Grosjean H. Nucleic Acids Res. 2006 Apr. 5; 34(6):1816-35. The RNA polymerase Ill-dependent family of genes in hemiascomycetes: comparative RNomics, decoding strategies, transcription and evolutionary implications.
Mouyna I, Henry C, Doering T L, Latgé J P. FEMS Microbiol Lett. 2004 Aug. 15; 237(2):317-24. Gene silencing with RNA interference in the human pathogenic fungus Aspergillus fumigatus.
Nakamura, Y., et al. Nucl. Acids Res. 2000 28:292. Codon usage tabulated from the international DNA sequence databases: status for the year 2000.
Nelson C E, Gersbach C A, Nat Biotechnol. 2016 Mar. 10; 34(3):298-9. Cas9 loosens its grip on off-target sites.
Nijland et al., Engineering of an endogenous hexose transporter into a specific D-xylose transporter facilitates glucose-xylose co-consumption in Saccharomyces cerevisiae, Biotechnol Biofuels 2014, 7, 168
Oliveira et al., Efficient cloning system for construction of gene silencing vectors in Aspergillus niger (2008) Appl. Microbiol. and Biotechnol. 80 (5): 917-924.
Ran F A, Hsu P D, Lin C Y, Gootenberg J S, Konermann S, Trevino A E, Scott D A, Inoue A, Matoba S, Zhang Y, Zhang F. Cell 2013 154, 1380-1389. Double nicking by RNA-guided CRISPR Cas9 for enhanced genome editing specificity.
Ran F A, Cong L, Yan W X, Scott D A, Gootenberg J S, Kriz A J, Zetsche B, Shalem O, Wu X, Makarova K S, Koonin E V, Sharp P A, Zhang F, Nature. 2015 Apr. 9; 520(7546):186-91. In vivo genome editing using Staphylococcus aureus Cas9.
Raymond C. K., Pownder T. A., Sexson S. L. Biotechniques 1999, 26: 134-141. General method for plasmid construction using homologous recombination.
Ryan et al., eLife, 2014, 3. doi: 10.7554/eLife.03703. Selection of chromosomal DNA libraries using a multiplex CRISPR system
Ryan O W, Skerker J M, Maurer M J, Li X, Tsai J C, Poddar S, Lee M E, DeLoache W, Dueber J E, Arkin A P, Cate J H. Elife. 2014. 19; 3. 03703.
Sambrook & Russell, Molecular Cloning: A Laboratory Manual, 3rd Ed., CSHL Press, Cold Spring Harbor, N.Y., 2001; and Ausubel et al., Current Protocols in Molecular Biology, Wiley InterScience, NY, 1995
Sander J D, Joung J K. Nat Biotechnol. 2014 April; 32(4):347-55. doi: 10.1038/nbt.2842. Epub 2014 Mar. 2. CRISPR-Cas systems for editing, regulating and targeting genomes.
Sikorski R S, Hieter P. Genetics. 1989 May; 122(1):19-27. A system of shuttle vectors and yeast host strains designed for efficient manipulation of DNA in Saccharomyces cerevisiae.
Slaymaker I M, Gao L, Zetsche B, Scott D A, Yan W X, Zhang F, Science. 2016 Jan. 1; 351(6268):84-8. Rationally engineered Cas9 nucleases with improved specificity.
Takahashi S, Nakajima Y, Imaizumi T, Furuta Y, Ohshiro Y, Abe K, Yamada R H, Kera Y. Development of an autonomously replicating linear vector of the yeast Cryptococcus humicola by using telomere-like sequence repeats. Appl Microbiol Biotechnol. 2011 February; 89(4):1213-21
Tsai S Q, Wyvekens N, Khayter C, et al. Nat Biotechnol. 2014 32(6):569-576. Dimeric CRISPR RNA-guided FokI nucleases for highly specific genome editing.
van Dijken J P, Bauer J, Brambilla L, Duboc P, Francois J M, Gancedo C, Giuseppin M L, Heijnen J J, Hoare M, Lange H C, Madden E A, Niederberger P, Nielsen J, Parrou J L, Petit T, Porro D, Reuss M, van Riel N, Rizzi M, Steensma H Y, Verrips C T, Vindelov J, Pronk J T. An interlaboratory comparison of physiological and genetic properties of four Saccharomyces cerevisiae strains. Enzyme Microb Technol. 2000 Jun. 1; 26(9-10):706-714.
Verwaal R, Wang J, Meijnen J P, Visser H, Sandmann G, van den Berg J A, van Ooyen A J. Appl Environ Microbiol. 2007 July; 73(13):4342-50. Epub 2007 May 11. High-level production of beta-carotene in Saccharomyces cerevisiae by successive transformation with carotenogenic genes from Xanthophyllomyces dendrorhous.
Wah, D. A.; J. Bitinaite, Schildkraut, I., Aggarwal, A. K. Proc Natl Acad Sci USA 1998 95 (18): 10564-9. Structure of FokI has implications for DNA cleavage.
Zetsche B, Gootenberg J S, Abudayyeh O O, Slaymaker I M, Makarova K S, Essletzbichler P, Volz S E, Joung J, van der Oost J, Regev A, Koonin E V, Zhang F, Cell. 2015 Oct. 22; 163(3):759-71. Cpfl is a single RNA-guided endonuclease of a class 2 CRISPR-Cas system.
Zhang G, Kong I I, Kim H, Liu J, Cate J H, Jin Y S. Appl Environ Microbiol. 2014 Dec. 15; 80(24):7694-701. doi: 10.1128/AEM.02310-14. Epub 2014 Oct. 3. Construction of a quadruple auxotrophic mutant of an industrial polyploidy Saccharomyces cerevisiae using RNA-guided Cas9 nuclease.

1. Method to produce a circular vector comprising one or more guide-polynucleotide expression cassettes, wherein said one or more guide-polynucleotide expression cassettes comprise a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following:
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette, wherein said fragment of a guide-polynucleotide expression cassette at the 5′-end and/or at the 3′-end of the polynucleotide sequence to be extended, does not comprise a polynucleotide coding for a guide sequence;
performing one or more overlap-extension PCR reactions by subjecting in each reaction one of said one or more polynucleotides and two suitable polynucleotide primers to yield one hybrid linear polynucleotide,
wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a circular vector comprising one or more functional guide-polynucleotide expression cassettes;
subjecting the one or more hybrid linear polynucleotides obtained in the overlap-extension PCR and optionally one or more additional linear polynucleotides to an assembly reaction yielding a circular vector comprising one or more functional guide-polynucleotide expression cassettes,
wherein the assembly reaction occurs in vivo,
optionally wherein the guide-polynucleotide is a gRNA.

2. The method according to claim 1 wherein the circular vector comprises two or more guide-polynucleotide expression cassettes.

3. The method according to claim 1, wherein the circular vector comprises one guide-polynucleotide expression cassette, optionally wherein the method comprises the following:
providing one polynucleotide, wherein said polynucleotide comprises a polynucleotide sequence to be extended, wherein the polynucleotide sequence to be extended comprises at the 5′-end and/or at the 3′-end to be extended a fragment of a guide-polynucleotide expression cassette;
performing one overlap-extension PCR reactions by subjecting in the reaction said polynucleotide and two suitable polynucleotide primers to yield one hybrid linear polynucleotide,
wherein the polynucleotide sequence to be extended and the suitable polynucleotide primers are selected so that the hybrid linear polynucleotide obtained in the overlap-extension PCR reaction comprises at least a polynucleotide coding for a guide sequence and suitable 5′-termini and 3′-termini which allow assembly in vivo of the hybrid linear polynucleotides and optionally of one or more additional linear polynucleotides with each other in a pre-defined order to yield a circular vector comprising one or more functional guide-polynucleotide expression cassettes;
subjecting the hybrid linear polynucleotide obtained in the overlap-extension PCR and optionally one or more additional linear polynucleotides to an assembly reaction yielding a circular vector comprising one or more functional guide-polynucleotide expression cassettes,
wherein the assembly reaction occurs in vivo,
optionally wherein the guide-polynucleotide is a gRNA.

4. The method according to claim 1 wherein the one or more polynucleotide sequence to be extended or additional linear polynucleotide comprises one or more elements selected from the group consisting of: an origin of replication or a fragment thereof, a selectable marker or a fragment thereof, a CAS9 expression cassette or a fragment thereof, a donor polynucleotide or a fragment thereof.

5. The method according to claim 4 wherein the circular vector obtained after assembly comprises one or more elements selected from the group consisting of: an origin of replication, a selectable marker, a CAS9 expression cassette, a donor polynucleotide or a combination of one or more of said elements, optionally wherein the circular vector obtained after assembly further comprises an origin of replication, optionally an origin of replication and a selectable marker.

6. A method of assembling a circular vector comprising one or more guide-polynucleotide expression cassettes in vivo, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following:
providing one or more linear polynucleotides, wherein said one or more linear polynucleotides comprise at the 5′-terminus and/or at the 3′-terminus at least one guide-polynucleotide expression cassette or a fragment of at least one guide-polynucleotide expression cassette comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a circular vector comprising one or more functional guide-polynucleotide expression cassettes;
optionally selecting a host cell comprising a circular vector and wherein said vector comprises one or more functional guide-polynucleotide expression cassettes;
optionally recovering the circular vector.

7. A method for producing a recombinant host cell comprising a circular vector, wherein said vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following:
providing one or more linear polynucleotides, wherein said one or more linear polynucleotides comprise at the 5′-terminus and/or at the 3′-terminus at least one guide-polynucleotide expression cassette or a fragment of at least one guide-polynucleotide expression cassette comprising at least the guide sequence;
transforming said one or more linear polynucleotides, and optionally one or more additional linear polynucleotides into a host cell;
allowing an homologous recombination reaction to take place between said one or more linear polynucleotides and optionally one or more additional linear polynucleotides, wherein said linear polynucleotides and additional linear polynucleotides have been selected to comprise suitable 5′-termini and 3′-termini which allow homologous recombination of said one or more linear polynucleotides and optionally of said one or more additional linear polynucleotides with each other in a pre-defined order to yield a circular vector comprising one or more functional guide-polynucleotide expression cassettes;
optionally selecting a host cell comprising a circular vector and wherein said vector comprises one or more functional guide-polynucleotide expression cassettes.

8. A method for producing a recombinant host cell according to claim 7 further comprising:
recovering the circular vector;
transforming the circular vector in a second host cell;
optionally isolating the second host cell comprising the circular vector.

9. A method for producing a recombinant host cell comprising a circular vector, wherein said vector comprises one or more guide-polynucleotide expression cassettes, wherein said one or more guide-polynucleotide expression cassette comprises a polynucleotide encoding a guide-polynucleotide operably linked to one or more control sequences which direct the expression of said guide-polynucleotide in a host cell, wherein said guide-polynucleotide comprises a guide-sequence that essentially is the reverse complement of a target-polynucleotide in a host cell and wherein the guide-polynucleotide can direct binding of a Cas protein at a target-polynucleotide in a host cell to form a CRISPR-Cas complex, wherein the method comprises the following:
performing a method of assembling a circular vector comprising one or more guide-polynucleotide expression cassettes in vivo according to claim 6 in a first host cell, optionally a first host cell belonging to the species S. cerevisiae, wherein said first host cell comprising the circular vector, is selected and the circular vector is recovered, wherein said vector comprises one or more functional guide-polynucleotide expression cassettes,
transforming the circular vector in a second host cell;
optionally isolating the second host cell comprising the circular vector.

10. A method according to claim 7 wherein the one or more linear polynucleotides are hybrid linear polynucleotides obtained by
providing one or more polynucleotides, wherein the one or more polynucleotides comprise a polynucleotide sequence to be extended, wherein the polynucleotide sequence to be extended comprises at the 5′-end and/or at the 3′-end to be extended a fragment of a guide-polynucleotide expression cassette, optionally wherein the fragment of a guide-polynucleotide expression cassette at the 5′-end and/or at the 3′-end of the polynucleotide sequence to be extended, does not comprise a polynucleotide coding for a guide sequence;
performing one or more overlap-extension PCR reactions by subjecting in each reaction one of said one or more polynucleotides and two suitable polynucleotide primers to yield one hybrid linear polynucleotide,
wherein the one or more polynucleotide sequence to be extended and the suitable polynucleotide primers are selected so that each hybrid linear polynucleotide obtained in the one or more overlap-extension PCR reactions comprises at least a polynucleotide coding for a guide sequence and suitable 5′-termini and 3′-termini which allow assembly in vivo of the one or more hybrid linear polynucleotides and optionally of one or more additional linear polynucleotides with each other in a pre-defined order to yield a circular vector comprising one or more functional guide-polynucleotide expression cassettes.

11. A method according to claim 10 wherein the one or more linear polynucleotides are two or more hybrid linear polynucleotides optionally obtained by:
providing two or more polynucleotides, wherein the two or more polynucleotides comprise a polynucleotide sequence to be extended, wherein the polynucleotide sequence to be extended comprises at the 5′-end and/or at the 3′-end to be extended a fragment of a guide-polynucleotide expression cassette, optionally wherein the fragment of a guide-polynucleotide expression cassette at the 5′-end and/or at the 3′-end of the polynucleotide sequence to be extended, does not comprise a polynucleotide coding for a guide sequence;
performing two or more overlap-extension PCR reactions by subjecting in each reaction one of said two or more polynucleotides and two suitable polynucleotide primers to yield one hybrid linear polynucleotide,
wherein the two or more polynucleotide sequence to be extended and the suitable polynucleotide primers are selected so that each hybrid linear polynucleotide obtained in the two or more overlap-extension PCR reactions comprises at least a polynucleotide coding for a guide sequence and suitable 5′-termini and 3′-termini which allow assembly in vivo of the two or more hybrid linear polynucleotides and optionally of one or more additional linear polynucleotides with each other in a pre-defined order to yield a circular vector comprising one or more functional guide-polynucleotide expression cassettes.

12. The method according to claim 10 wherein the one or more linear polynucleotides is one hybrid linear polynucleotide optionally obtained by:
providing one polynucleotide, wherein said polynucleotide comprises a polynucleotide sequence to be extended, wherein the polynucleotide sequence to be extended comprises at the 5′-end and/or at the 3′-end to be extended a fragment of a guide-polynucleotide expression cassette, optionally wherein the fragment of a guide-polynucleotide expression cassette at the 5′-end and/or at the 3′-end of the polynucleotide sequence to be extended, does not comprise a polynucleotide coding for a guide sequence;
performing one overlap-extension PCR reactions by subjecting in the reaction said polynucleotide and two suitable polynucleotide primers to yield one hybrid linear polynucleotide,
wherein the polynucleotide sequence to be extended and the suitable polynucleotide primers are selected so that the hybrid linear polynucleotide obtained in the overlap-extension PCR reaction comprises at least a polynucleotide coding for a guide sequence and suitable 5′-termini and 3′-termini which allow assembly in vivo of the hybrid linear polynucleotides and optionally of one or more additional linear polynucleotides with each other in a pre-defined order to yield a circular vector comprising one or more functional guide-polynucleotide expression cassettes.

13. A method according to claim 1, wherein the assembly reaction occurs in a host cell belonging to S. cerevisiae species.

14. A method according to claim 8 wherein the second host cell is a prokaryotic, optionally a bacterial host cell, or an eukaryotic host cell, optionally a fungal host cell.

15. The method according to claim 1 wherein the circular vector comprises at least 2 or more functional guide polynucleotide expression cassettes.

16. A circular vector comprising one or more functional guide polynucleotide expression cassettes obtainable by the method of claim 1.

17. A recombinant host cell comprising a circular vector comprising one or more functional guide polynucleotides expression cassettes which recombinant host cell is obtainable by the method of claim 7.

18. A non-naturally occurring or engineered composition comprising a source of a CRISPR-Cas system comprising a guide-polynucleotide and a Cas protein, wherein the guide-polynucleotide comprises a guide-sequence that essentially is the reverse complement of a target-polynucleotide in a host cell and wherein the guide-polynucleotide can direct binding of the Cas protein at the target-polynucleotide in the host cell to form a CRISPR-Cas complex, wherein the guide-sequence is essentially the reverse complement of the (N)y part of a 5′-(N)yPAM-3′ polynucleotide sequence target in the genome of the host cell, wherein y is an integer of 8-30, wherein PAM is a protospacer adjacent motif, wherein the host cell is optionally a prokaryote or a eukaryote, and wherein PAM is optionally a sequence selected from the group consisting of 5′-XGG-3′, 5′-XGGXG-3′, 5′-XXAGAAW-3′, 5′-XXXXGATT-3′, 5′-XXAGAA-3′, 5′-XAAAAC-3′, wherein X can be any nucleotide or analog thereof, optionally X can be any nucleotide; and W is A or T, wherein the guide-polynucleotide in said composition and optionally a Cas protein in said composition is comprised in a circular vector according to claim 16.

19. Method of modulating expression of a polynucleotide in a host cell, comprising contacting a host cell with the composition according to claim 18, wherein the guide-polynucleotide directs binding of the Cas protein at the target-polynucleotide in the host cell to form a CRISPR-Cas complex, optionally wherein the host cell comprises a polynucleotide encoding a compound of interest.

20. Method for the production of a compound of interest, comprising culturing under conditions conducive to the production of the compound of interest a recombinant host cell according to claim 17 and optionally purifying or isolating the compound of interest.
